(19) United States
(12) Patent Application Publication
(10) Pub. No.: US 2026/0276643 A1
(43) Pub. Date: Sep. 17, 2026

(54) METHODS FOR PREDICTING AND IMPROVING THERAPEUTIC EFFICACY OF CANCER TREATMENTS AND METHODS FOR CANCER PROGNOSIS

(71) Applicants: Università degli studi di Verona, Verona (IT); BioNTech SE, Mainz (DE)

(72) Inventors: Francesco DE SANCTIS, Verona (IT); Vincenzo BRONTE, Verona (IT); Michael ERDELJAN, Mainz (DE)

(21) Appl. No.: 19/471,247

(22) PCT Filed: Mar. 27, 2024
(86) PCT No.: PCT/EP2024/058240

§ 371 Date: Sep. 30, 2025

(30) Foreign Application Priority Data
Mar. 31, 2023 (WO) PCT/EP2023/058504

(51) Int. Cl. G01N 33/575

(52) U.S. Cl. CPC G01N 33/5759; G01N 2333/705; G01N 2800/52; G01N 2800/7028

(57) ABSTRACT

The present invention provides a prognostic marker in human cancer, CLDN18. Methods involving this marker are disclosed for predicting therapeutic efficacy of cancer treatments and the prognosis of cancer. The present invention also provides a method for selecting a subject affected with a cancer for an immunotherapy. Furthermore, the present invention provides a therapy for treating cancer.

### TECHNICAL FIELD

The present invention provides a prognostic marker in human cancer, CLDN18. Methods involving this marker are disclosed for predicting therapeutic efficacy of cancer treatments and the prognosis of cancer. The present invention also provides a method for selecting a subject affected with a cancer for an immunotherapy. Furthermore, the present invention provides a therapy for treating cancer.

### BACKGROUND

Cancer is the second leading cause of death globally and is expected to be responsible for an estimated 9.6 million deaths in 2018 (Bray, F. et al. (2018) CA: A Cancer Journal for Clinicians, 68: 394-424). In general, once a solid tumor has metastasized, with a few exceptions such as germ cell and some carcinoid tumors, 5-year survival rarely exceeds 25%. Conventional therapies such as chemotherapy, radiotherapy, surgery, and targeted therapies and recent advances in immunotherapies have improved outcomes in patients with advanced solid tumors. In the last few years, the Food and Drug Administration (FDA) and European Medicines Agency (EMA) have approved eight checkpoint inhibitors (one monoclonal antibody targeting the CTLA-4 pathway, ipilimumab, and seven antibodies targeting programmed death receptor/ligand [PD/PD-L1], including atezolizumab, avelumab, durvalumab, nivolumab, cemiplimab and pembrolizumab), for the treatment of patients with multiple cancer types, mainly solid tumors. These approvals have dramatically changed the landscape of cancer treatment. However, certain cancers such as pancreatic adenocarcinoma still do not yet benefit from existing therapies including immunotherapies.

Pancreatic ductal adenocarcinoma (PDAC) is a clinical challenge (Hosein, A. N. et al. (2022) Nat Cancer 3, 272-286). Late diagnosis, small surgery opportunities, frequent tumor recurrences, and limited chemotherapy efficacy pose overwhelming hurdles. In addition, immune therapy, which provides encouraging results in a multitude of solid tumors, has shown limited efficacy in the context of PDAC (O'Reilly, E. M. et al. (2019) JAMA Oncol 5, 1431-1438). These unique features restrict the median patient survival to about two years (Neoptolemos, J. P. et al. (2018) Nat Rev Gastroenterol Hepatol 15, 333-348) and project pancreatic cancer as the second leading cause of cancer-related mortality in the near future (Rahib, L. et al. (2014) Cancer Res 74, 2913-2921; Sung, H. et al. (2021) CA Cancer J Clin 71, 209-249). Increased understanding of pancreatic cancer evolution at genetic, transcriptomic, metabolic, stromal and immunological levels uncovered some drivers of these failures. The therapeutic response to immune checkpoint inhibitors often relies on T cell infiltration (Bruni, D. et al. (2020) Nat Rev Cancer 20, 662-680; Tumeh, P. C. et al. (2014) Nature 515, 568-571), and tumor mutational burden (McGranahan, N. et al. (2016) Science 351, 1463-1469; Rizvi, N. A. et al. (2015) Science 348, 124-128; Snyder, A. et al. (2014) N Engl J Med 371, 2189-2199) which are both limited in pancreatic cancer (Alexandrov, L. B. et al. (2013) Nature 500, 415-421). Accordingly, immunotherapy showed clinical benefits only in a small fraction (about 1%) of PDAC patients, characterized by DNA mismatch repair alterations (Le, D.T. et al. (2017) Science 357, 409-413). Molecular fingerprinting rather than histology analysis recently provided a more accurate classification of pancreatic cancers into distinct subgroups with peculiar features, which can be associated with more aggressive progression and worse clinical survival (Collisson, E. A. et al. (2019) Nat Rev Gastroenterol Hepatol 16, 207-220). Identification of common genetic alterations in pancreatic cancer, such as KRAS (G12D) paved the way towards the development of selective inhibitors and employment of transgenic TCR-engineered cytotoxic T lymphocytes (CTLs) targeting this mutation; the two strategies showed promising results in both preclinical and human settings, respectively (Leidner et al., (2022) N Engl J Med 386, 2112-2119; Mao et al., (2022) Cell Discov 8, 5). Furthermore, molecular deconvolution of the tumor immune microenviromnent also revealed that local immune suppression at baseline predicts patients with worse response to chemotherapy and immune therapy in pancreatic cancer (Padron, L. J. et al. (2022) Nat Med 28, 1167-1177).

Pancreatic cancer cells cooperate with immune regulatory cells, endothelial cells and fibroblasts to establish a peculiar hostile network that hides tumors from immune recognition and containment (Balachandran, V. P. et al. (2019) Gastroenterology 156, 2056-2072). The immunosuppressive tumor microenvironment (TME) results in ineffective priming of tumor-specific adaptive immune responses (Hegde, S. et al. (2020) Cancer Cell 37, 289-307 e289) and poor infiltration and fitness of CTLs (Clark, C. E. et al. (2007) Cancer Res 67, 9518-9527; De Sanctis, F. et al. (2022) J Immunother Cancer 10). Nonetheless, the common viewpoint of pancreatic cancer as an immune desert has been recently reconsidered. New findings suggest that the TME can be manipulated to restore efficient antigen presentation (Beatty, G. L. et al. (2011) Science 331, 1612-1616) reshaping myeloid function toward an antitumor phenotype (Panni, R. Z. et al. (2019) Sci Transl Med 11) and laying the groundwork for chemo-immunotherapy efficacy (Padron, L. J. et al. (2022) Nat Med 28, 1167-1177).

Currently, the methods to determine prognosis and select patients for adjuvant therapy rely mainly on pathological and clinical staging. However, due to insufficiently accurate prognosis predictions, a substantial proportion of cancer subjects receive adjuvant systemic therapy without gaining any benefit.

Therefore, there is a great need for the identification of prognostic markers that can accurately distinguish tumors which are associated with good prognosis or therapeutic efficiency from others which are not. Using such markers, the practitioner can predict the patient's prognosis and can effectively target the individuals who would most likely benefit from adjuvant therapy, e.g., immunotherapy.

Here, we unveil an unrecognized function for CLDN18 in regulating host cancer immune surveillance. By integrating molecular, phenotypical, functional and in vivo data, we identified the claudin family as a new class of molecules acting as extrinsic tumor suppressor elements by regulating the immune landscape of TME and, in particular, tumor-CTL physical interactions. We unveiled CLDN18 as a forefather of a new class of proteins, describing in a mechanistic way its role in dictating T-cell entrance into tumors and supporting T-cell activation, and confirmed its prognostic value in pancreatic and lung adenocarcinoma patients.

Claudins (CLDNs) are membrane tetraspanins orchestrating tight junction formation and maintenance of tissue architectural and biological functions in many organs. Despite its etymology (in Latin, “claudere” means “to close”), the 27 mammalian members of the CLDN family carry out different functions, establishing para-cellular barriers with defined characteristics, including selective channel function for ions and small hydrophobic molecules, and unique microenvironments associated with different physiologic processes (Suzuki, H. et al. (2014) Science 344, 304-307). Accordingly, preclinical studies ablating CLDNs' expression revealed their importance in maintaining water balance and preventing inflammation and the occurrence of diseases, including cancer (Tsukita, S. et al. (2019) Trends Biochem Sci 44, 141-152). Although many elements of this family can potentially behave as either oncogenes or oncosuppressor genes, their role in cancer progression is still debated and possibly context dependent (Li, J. (2021). Front Oncol 11, 676781). CLDNs are often deregulated during neoplastic progression, as in the case of CLDN18, whose expression in healthy individuals is restricted to the lung (CLDN18.1 variant) and stomach (CLDN18.2 variant). CLDN18.2-dependent tight junctions impede proton leakage from gastric mucosa, and CLDN18.2 downregulation is associated with inflammation and stomach cancer (Hagen, S. J. et al. (2018) Gastroenterology 155, 1852-1867; Hayashi, D. et al. (2012) Gastroenterology 142, 292-304). However, the gene can be activated during neoplastic evolution of pancreatic cancer (Woll, S. et al. (2014) Int J Cancer 134, 731-739). Thus, CLDNs are potential targets for diagnosis, prognosis and cancer treatment (Qi, C. et al. (2022) Nat Med 28, 1189-1198; Reinhard, K. et al. (2020) Science 367, 446-453.). Notably, few reports acknowledge a direct, immune-regulating role for CLDNs, thus far limited to the innate immune defense arm (Tsai, P. Y. et al. (2017) Cell Host Microbe 21, 671-681 e674).

The findings presented herein may be used for cancer prognosis and to select a suitable treatment for a cancer patient and, in particular, to decide whether immunotherapy should be administered to a cancer patient.

### SUMMARY

The present invention provides methods to measure the eligibility of patients for certain cancer treatments, in particular immunotherapy, and to draw conclusions on the prognosis of a cancer patient. The results obtained using these tests enables the physician to decide on a suitable treatment for a cancer patient, and, in particular, to decide whether immunotherapy should be administered to a particular cancer patient.

An association of human claudin 18 (CLDN18) with immune system efficacy, in particular T lymphocyte efficacy, in cancer and the prognostic value of CLDN18 in cancer is demonstrated herein.

High CLDN18 expression levels correlate with good efficacy of cancer treatments and good prognosis of cancer, in particular good overall or disease free survival. Low CLDN18 expression levels correlate with poor efficacy of cancer treatments and poor prognosis of cancer, in particular poor overall or disease free survival.

CLDN18 thus represents a marker, which is relevant for the prognosis in cancer and may be used herein for predicting or monitoring clinical outcome of a subject affected with cancer.

Accordingly, the present invention concerns a method (i) for determining the ability of a subject affected with cancer to mount a natural or immunotherapy-induced immune response against the cancer, (ii) for determining the survival perspective of a subject affected with cancer, (iii) for determining the responsiveness of a subject affected with cancer to immunotherapy, or (iv) any combination of (i), (ii) and (iii). The method comprises determining the expression level of claudin 18 (CLDN18) in a sample obtained from the subject, e.g., in a cancer sample obtained from the subject.

A low expression level of CLDN18 is indicative of a poor prognosis or clinical outcome, either without or with immunotherapeutic treatment. A high expression level of CLDN18 is indicative of a good prognosis or clinical outcome, either without or with immunotherapeutic treatment. In some embodiments, a poor prognosis is a decreased survival and/or an early disease progression and/or an increased disease recurrence and/or an increased metastasis formation. Specifically, a low expression level of CLDN18 is indicative of the poor survival perspective of a subject affected with cancer. A high expression level of CLDN18 is indicative of the good survival perspective of a subject affected with cancer.

A low expression level of CLDN18 is indicative of a poor ability of a subject affected with cancer to mount a natural or immunotherapy-induced immune response against the cancer. A high expression level of CLDN18 is indicative of a good ability of a subject affected with cancer to mount a natural or immunotherapy-induced immune response against the cancer.

A low expression level of CLDN18 is indicative of a poor responsiveness of a subject affected with cancer to immunotherapy. A high expression level of CLDN18 is indicative of a good responsiveness of a subject affected with cancer to immunotherapy.

In a further aspect, the present invention concerns a method for selecting a subject affected with cancer for immunotherapy, or determining whether a subject affected with cancer is susceptible to benefit from immunotherapy. The method comprises determining the expression level of claudin 18 (CLDN18) in a sample obtained from the subject, e.g., in a cancer sample obtained from the subject, a high expression level of CLDN18 indicating that immunotherapy is indicated or required.

In addition, increasing claudin 18 (CLDN18) expression was shown to support the efficacy of immunotherapy.

Accordingly, in a further aspect, the present invention concerns a method for improving responsiveness of a subject affected with cancer to immunotherapy, or improving the possibility of a subject affected with cancer to benefit from immunotherapy. The method comprises administering an agent stabilizing or increasing expression of CLDN18 to the subject.

In one aspect the present invention provides a method for determining the ability of a cancer patient to mount a natural or immunotherapy-induced immune response against the cancer comprising determining the expression level of claudin 18 (CLDN18) in a sample obtained from the patient.

In some embodiments, the immune response involves anti-tumor T cells.

In some embodiments, the immune response comprises targeting an antigen other than CLDN18.

In some embodiments, the antigen other than CLDN18 is a tumor antigen.

In some embodiments, expression of CLDN18 indicates that the patient is able to mount a natural or immunotherapy-induced immune response against the cancer.

In some embodiments, a level of CLDN18 at or above a reference level indicates that the patient is able to mount a natural or immunotherapy-induced immune response against the cancer.

In some embodiments, the method is for determining the survival perspective of the cancer patient.

In some embodiments, expression of CLDN18 indicates that the patient has a favorable survival perspective.

In some embodiments, a level of CLDN18 at or above a reference level indicates that the patient has a favorable survival perspective.

In some embodiments, the method is for determining the responsiveness of the cancer patient to immunotherapy.

In some embodiments, the immunotherapy aims at inducing an immune response involving anti-tumor T cells.

In some embodiments, the immunotherapy aims at enhancing T cell tumor infiltration and/or the number and/or activity of anti-tumor T cells.

In some embodiments, the immunotherapy is selected from the group consisting of immune checkpoint blockade, T cell stimulation, cancer vaccines, oncolytic viruses, antigen-presenting cell activation, adoptive T cell therapies and CAR T cell therapies.

In some embodiments, the immunotherapy comprises targeting an antigen other than CLDN18.

In some embodiments, the antigen other than CLDN18 is a tumor antigen.

In some embodiments, expression of CLDN18 indicates that the patient responds to immunotherapy.

In some embodiments, a level of CLDN18 at or above a reference level indicates that the patient responds to immunotherapy.

In a further aspect the present invention provides a method for determining the survival perspective of a cancer patient comprising determining the expression level of claudin 18 (CLDN18) in a sample obtained from the patient.

In some embodiments, expression of CLDN18 indicates that the patient has a favorable survival perspective.

In some embodiments, a level of CLDN18 at or above a reference level indicates that the patient has a favorable survival perspective.

In a further aspect the present invention provides a method for determining the responsiveness of a cancer patient to immunotherapy comprising determining the expression level of claudin 18 (CLDN18) in a sample obtained from the patient.

In some embodiments, the immunotherapy aims at inducing an immune response involving anti-tumor T cells.

In some embodiments, the immunotherapy aims at enhancing T cell tumor infiltration and/or the number and/or activity of anti-tumor T cells.

In some embodiments, the immunotherapy is selected from the group consisting of immune checkpoint blockade, T cell stimulation, cancer vaccines, oncolytic viruses, antigen-presenting cell activation, adoptive T cell therapies and CAR T cell therapies.

In some embodiments, the immunotherapy comprises targeting an antigen other than CLDN18.

In some embodiments, the antigen other than CLDN18 is a tumor antigen.

In some embodiments, expression of CLDN18 indicates that the patient responds to immunotherapy.

In some embodiments, a level of CLDN18 at or above a reference level indicates that the patient responds to immunotherapy.

In some embodiments of all aspects described herein, the sample comprises cancer cells.

In some embodiments of all aspects described herein, the sample comprises a tumor biopsy.

In a further aspect the present invention provides a method for treating a cancer patient, the method comprising:
(i) determining the expression level of claudin 18 (CLDN18) in a sample obtained from the patient and
(ii) administering an immunotherapy to the patient, if the patient is found to be eligible in (i).

In some embodiments, if CLDN18 is expressed, the immunotherapy is administered to the patient.

In some embodiments, if the level of CLDN18 is at or above a reference level, the immunotherapy is administered to the patient.

In some embodiments, if CLDN18 is not expressed, the immunotherapy is not administered to the patient or is administered in combination with an agent stabilizing or increasing expression of CLDN18.

In some embodiments, if the level of CLDN18 is below a reference level, the immunotherapy is not administered to the patient or is administered in combination with an agent stabilizing or increasing expression of claudin 18 (CLDN18).

In a further aspect the present invention provides a method for treating a cancer patient, the method comprising:
(i) determining that a sample obtained from the patient has an expression level of claudin 18 (CLDN18) at or above a reference level and
(ii) administering an immunotherapy to the patient.

In some embodiments, the immunotherapy aims at inducing an immune response involving anti-tumor T cells.

In some embodiments, the immunotherapy aims at enhancing T cell tumor infiltration and/or the number and/or activity of anti-tumor T cells.

In some embodiments, the immunotherapy is selected from the group consisting of immune checkpoint blockade, T cell stimulation, cancer vaccines, oncolytic viruses, antigen-presenting cell activation, adoptive T cell therapies and CAR T cell therapies.

In some embodiments, the immunotherapy comprises targeting an antigen other than CLDN18.

In some embodiments, the antigen other than CLDN18 is a tumor antigen.

In some embodiments, the sample comprises cancer cells.

In some embodiments, the sample comprises a tumor biopsy.

In a further aspect the present invention provides a method for enhancing the ability of a cancer patient to mount a natural or immunotherapy-induced immune response against the cancer comprising administering an agent stabilizing or increasing expression of claudin 18 (CLDN18).

In some embodiments, the immune response involves anti-tumor T cells.

In some embodiments, the immune response comprises targeting an antigen other than CLDN18.

In some embodiments, the antigen other than CLDN18 is a tumor antigen.

In some embodiments, the immunotherapy aims at inducing an immune response involving anti-tumor T cells.

In some embodiments, the immunotherapy aims at enhancing T cell tumor infiltration and/or the number and/or activity of anti-tumor T cells.

In some embodiments, the immunotherapy is selected from the group consisting of immune checkpoint blockade, T cell stimulation, cancer vaccines, oncolytic viruses, antigen-presenting cell activation, adoptive T cell therapies and CAR T cell therapies.

In some embodiments, the immunotherapy comprises targeting an antigen other than CLDN18.

In some embodiments, the antigen other than CLDN18 is a tumor antigen.

In a further aspect the present invention provides a method for enhancing the responsiveness of a cancer patient to immunotherapy comprising administering an agent stabilizing or increasing expression of claudin 18 (CLDN18).

In a further aspect the present invention provides a method for treating a cancer patient, the method comprising:
(i) administering an agent stabilizing or increasing expression of claudin 18 (CLDN18) and
(ii) administering an immunotherapy to the patient.

In some embodiments, the immunotherapy aims at inducing an immune response involving anti-tumor T cells.

In some embodiments, the immunotherapy aims at enhancing T cell tumor infiltration and/or the number and/or activity of anti-tumor T cells.

In some embodiments, the immunotherapy is selected from the group consisting of immune checkpoint blockade, T cell stimulation, cancer vaccines, oncolytic viruses, antigen-presenting cell activation, adoptive T cell therapies and CAR T cell therapies.

In some embodiments, the immunotherapy comprises targeting an antigen other than CLDN18.

In some embodiments, the antigen other than CLDN18 is a tumor antigen.

In some embodiments, the methods described herein may comprise determining the expression level of claudin 18 (CLDN18) in a sample obtained from the subject (or patient), e.g., in a cancer sample obtained from the subject, prior to administering an agent stabilizing or increasing expression of CLDN18 to the subject. In some embodiments, an agent stabilizing or increasing expression of CLDN18 is administered to the subject if an insufficient expression level of claudin 18 (CLDN18) has been determined in a sample obtained from the subject, e.g., in a cancer sample obtained from the subject.

In a further aspect the present invention provides a method for treating a cancer patient, the method comprising:
(i) determining the expression level of claudin 18 (CLDN18) in a tumor biopsy sample obtained from the patient
(ii) comparing the expression level of CLDN18 to a reference, and
(iii) administering an immunotherapy to the patient when the expression level of CLDN18 is higher than the reference.

In a further aspect the present invention provides a method for treating a cancer patient, the method comprising administering immunotherapy to a patient having been determined to have a tumor that expresses claudin 18 (CLDN18).

In a further aspect the present invention provides a method for treating a cancer patient, the method comprising administering immunotherapy to a patient having been determined to have a tumor that expresses claudin 18 (CLDN18) at a level higher than a reference.

In a further aspect the present invention provides a method of increasing therapeutic T-cell migration to a tumor in the patient, the method comprising administering to the patient an agent that stabilizes or increases expression of claudin 18 (CLDN18).

In a further aspect the present invention provides a method for treating a tumor in a patient, the method comprising administering to the patient immunotherapy in combination with an agent that stabilizes or increases expression of claudin 18 (CLDN18) if the patient has been determined to have a tumor that does not express CLDN18 or that expresses CLDN18 at a level lower than a reference.

In some embodiments, the immunotherapy in any of the aspects defined above aims at inducing an immune response involving anti-tumor T cells.

In some embodiments, the immunotherapy in any of the aspects defined above aims at enhancing T cell tumor infiltration and/or the number and/or activity of anti-tumor T cells.

In some embodiments, the immunotherapy in any of the aspects defined above is selected from the group consisting of immune checkpoint blockade, T cell stimulation, cancer vaccines, oncolytic viruses, antigen-presenting cell activation, adoptive T cell therapies and CAR T cell therapies.

In some embodiments, the immunotherapy in any of the aspects defined above comprises targeting an antigen other than CLDN18.

In some embodiments, the antigen other than CLDN18 is a tumor antigen.

In any of the aspects described herein, the agent that stabilizes or increases expression of CLDN18 may be administered prior to, simultaneously with or subsequently to the immunotherapy.

In any of the aspects described herein, the cancer comprises pancreatic cancer.

In any of the aspects described herein, the cancer comprises lung cancer.

In any of the aspects described herein, the cancer comprises lung adenocarcinoma.

In some embodiments of all aspects described herein, the expression level of CLDN18 is determined by measuring the quantity of CLDN18 protein or CLDN18 mRNA.

In some embodiments, the quantity of CLDN18 protein is measured by immuno-histochemistry, semi-quantitative Western-blot or by protein or antibody arrays.

In some embodiments, the quantity of CLDN18 mRNA is measured by quantitative or semi-quantitative RT-PCR, or by real time quantitative or semi-quantitative RT-PCR or by transcriptome approaches.

In some embodiments, the methods described herein comprise the step of comparing the expression level of CLDN18 to a reference expression level. Additionally, the methods described herein further comprise the step of determining whether the expression level of CLDN18 is high(er) or low(er) compared to said reference expression level.

In some embodiments, the reference expression level is the expression level of CLDN18 in one or more samples obtained from one or more subjects (i) having an ability to mount a natural or immunotherapy-induced immune response against cancer, (ii) having a favourable survival perspective, (iii) having an ability to respond to immunotherapy, or (iv) any combination of (i), (ii) and (iii). In some embodiments, a level of CLDN18 at or above the reference level is a high expression level of CLDN18. In some embodiments, a level of CLDN18 at or above the reference level indicates that a subject affected with cancer (i) has the ability to mount a natural or immunotherapy-induced immune response against the cancer, (ii) has a good survival perspective, (iii) has the ability to respond to immunotherapy, or (iv) any combination of (i), (ii) and (iii). In some embodiments, a high expression level of CLDN18 indicates that a subject affected with cancer (i) has the ability to mount a natural or immunotherapy-induced immune response against the cancer, (ii) has a good survival perspective, (iii) has the ability to respond to immunotherapy, or (iv) any combination of (i), (ii) and (iii). In some embodiments, a level of CLDN18 below the reference level is a low expression level of CLDN18. In some embodiments, a level of CLDN18 below the reference level indicates that a subject affected with cancer (i) does not have the ability to mount a natural or immunotherapy-induced immune response against the cancer, (ii) does not have a good survival perspective, (iii) does not have the ability to respond to immunotherapy, or (iv) any combination of (i), (ii) and (iii). In some embodiments, a low expression level of CLDN18 indicates that a subject affected with cancer (i) does not have the ability to mount a natural or immunotherapy-induced immune response against the cancer, (ii) does not have a good survival perspective, (iii) does not have the ability to respond to immunotherapy, or (iv) any combination of (i), (ii) and (iii).

In addition, the present invention concerns the use of CLDN18 as a prognostic marker in cancer, as a marker for selecting a subject affected with cancer for immunotherapy, or for determining whether a subject affected with a cancer is susceptible to benefit from immunotherapy.

In some embodiments, the cancer is a solid cancer or a hematopoietic cancer, preferably a solid cancer. Preferably, the cancer is selected from the group consisting of breast cancer, osteosarcoma, skin cancer, ovarian cancer, lung cancer, liver cancer, cervix cancer, liposarcoma, gastric cancer, pancreatic cancer, bladder cancer, vulvar cancer, colon cancer and brain cancer. More preferably, the cancer is lung cancer or pancreatic cancer. Even more preferably, the cancer is pancreatic cancer such as early stage pancreatic cancer.

The present invention also concerns a kit for predicting or monitoring clinical outcome of a subject affected with a cancer, for selecting a subject affected with cancer for immunotherapy, and/or for determining whether a subject affected with cancer is susceptible to benefit from immunotherapy, wherein the kit comprises (i) at least one antibody specific to CLDN18 and/or (ii) at least one probe specific to the CLDN18 mRNA or cDNA and/or (iii) at least one nucleic acid primer pair specific to CLDN18 mRNA or cDNA and, optionally, a leaflet providing guidelines to use such a kit. Preferably, the kit further comprises means for detecting the formation of the complex between CLDN18 and said at least one antibody specific to CLDN18 and/or means for detecting the hybridization of said at least one probe specific to the CLDN18 mRNA or cDNA on CLDN18 mRNA or cDNA and/or means for amplifying and/or detecting said CLDN18 mRNA or cDNA.

Other objects, advantages and features of the present invention will become apparent from the following detailed description when considered in conjunction with the accompanying figures.

### BRIEF DESCRIPTION OF THE DRAWINGS

FIG. 1. FC1242-OVA and FC1199-OVA recapitulate cold and hot pancreatic adenocarcinoma tumors. A) Tumor growth in immunocompetent mice bearing FC 1199, FC1199-OVA, FC1242 and FC1242-OVA s.c. tumors (n=5/group). B) Tumor growth curve in immunodeficient mice bearing either FC1199-OVA or FC1242-OVA tumors, treated or not with adoptive transfer of OVA-specific CTL. Mice treated as in (B) were euthanized 3 days following ACT to quantify CTLs by FC in spleen (C, left panel) and in tumors, either by FC (C, right panel) or IHC (D, right panel). TILs identified in a heatmap plot using a color code according to their number in representative tumor sections (D, left panel). E) GSVA scores of FC1242-OVA and FC1199-OVA on PDAC molecular subtypes (Bailey et al., (2016) Nature 531, 47-52) (Top panel A: ADEX, I: Immunogenic, P: Progenitor, S: Squamous). GSVA scores on immune phenotypic and functional gene signatures (Rooney et al., (2015) Cell 160, 48-61) were compared between FC1242-OVA and FC1199-OVA cell lines (lower panel). F) FC1242-OVA and FC1199-OVA cell lines were compared with the KPC-derived cell lines characterized as TILs^{hi} and TILs^{low} calculating the GSVA scores on the immunogenic PDAC signature (top panel). FC1242-OVA and FC1199-OVA molecular signatures were directly compared with GSVA on the signatures of TILs^{hi} and TILs^{low} cell lines (lower panel).

FIG. 2. Hot tumors are endowed with unique molecular signature associated with biological adhesion processes and CLDN expression. A-B) Gene ontology (GO) pathway enrichment analyses of biological processes (upper panels) and cellular components (lower panels) identified in hot (A) and cold (B) tumors. C) CLDN18 expression on TCGA PAAD dataset annotated with the PDAC molecular subtypes according to Bailey [(Bailey et al., (2016) Nature 531, 47-52); left panel], Collisson [(Collisson et al., (2011) Nat Med 17, 500-503); central panel] and Moffitt [(Moffitt et al., (2015) Nat Genet 47, 1168-1178); right panel)]. D-E) Linear associations between CLDN18 and neoplastic, adhesion and immune processes from biological pathways. F) CLDN18 expression in molecular subtypes of NSCLC (Chen et al., (2017), Oncogene 36, 1384-1393.). G) Scatter plot showing linear association and Spearman's correlation between CLDN18 expression and CD3E in LUAD. H) Kaplan-Meier curves showing the overall survival of LUAD patients based on CLDN18 and CD3E expression. I) FC assessment of CLDN18 expression in FC1199-OVA and FC1242-OVA cell lines. (J) Representative IHC images of FC1199-OVA- and FC1242-OVA-derived tumors isolated from ACT-treated immunodeficient mice, showing CLDN18 (green) and TILs (red); scale bar=500 μm.

FIG. 3. CLDN18 ablation reduces tumor hotness. Immunodeficient mice bearing FC1199-OVA CLDN18^{+/+} and FC1199-OVA CLDN18^{-/-} tumors, treated or not with OVA-specific CTL ACT, were euthanized 3 days following the ACT to quantify CTLs by FC (A) or IHC (B, scale bar: 100 μm). C) TILs localization in tumor mass according to the distance from the core. D) RT-PCR for Tnf-a and Gzm-B on mRNA extracted from whole tumors of mice treated with the adoptive transfer of OVA-specific CTL. E) Tumor growth in immunodeficient mice bearing FC1199-OVA CLDN18^{+/+} and FC1199-OVA CLDN18^{-/-} tumors, treated with OVA-specific CTLs. TILs were quantified in DANG and DANG-CLDN18 tumors 3 days after adoptive transfer of h-TERT specific CTLs by FC (F) and IHC (G): “heatmap” plots, left panel; CD3 quantification, right panel). HT) Tumor growth curves in immunodeficient mice bearing either DANG or DANG-CLDN18 tumors and treated or not with hTERT-specific CTLs (n=5/group).

FIG. 4. CLDN18 expression on tumor cells supports interaction with and activation of T lymphocytes. A) CTL intra-tumor in vivo motility parameters in FC1199-OVA CLDN18+/+ or CLDN18−/− tumor cells. B) CD8+ CTL trajectories in CLDN18+/+ and CLDN18−/− tumors from starting point (normalized as 0) and plotted in a bi-dimensional graph. C) In vitro kinetics of CTL interactions (parameters: contact time in minutes, and arrest index) with FC1199-OVA CLDN18+/+ and CLDN18−/− tumor cells. D) CTLs were cultured with FC1199-OVA CLDN18+/+ and CLDN18−/− tumor cells at 1/1/1 ratio. Percentage of CTLs establishing IS with FC1199-OVA CLDN18+/+ and CLDN18−/− tumor cells. E) Kinetic analysis of IS formation in CTLs co-cultured with FC1199-OVA CLN18+/+ or CLDN18−/− tumor cells (evaluated as intracellular Ca2+ spikes). F) IFN-γ quantification on the supernatant of CTLs co-cultured with either FC1199-OVA CLDN18+/+ or FC1199-OVA CLDN18−/− tumor cells. G) Kinetics of OVA-specific CTL killing of FC1199-OVA CLDN18+/+ or FC1199-OVA CLDN18−/− tumor cells. Values are normalized on untreated tumor cells.

FIG. 5. CLDN18 supports ALCAM localization on cell membrane and its accrual in lipid rafts. A) FC quantification of adhesion molecules on the cell membrane of FC1199-OVA and FC1242-OVA tumor cells. B) FC quantification of ALCAM cell surface expression on FC1242-OVA, FC1199-OVA and FC1199-derived clones engineered to lack or expressing CLDN18. C) IF quantification of ALCAM surface expression on FC1199-OVA CLDN18^{+/+} or FC1199-OVA CLDN18/cell clones. D) Immunoblot of total ALCAM protein evaluation in FC 1199-OVA clones and its quantification. E) Localization of ALCAM, Actin and CLDN18 to lipid rafts of FC1199-OVA CLDN18^{+/+} (left panel) and FC1199-OVA CLDN18/(right panel) tumor cells. Lipid rafts were identified in fractions 3 and 4 of density gradient separation (peaks of cholesterol, phospholipids and presence of caveolin 1 [CAV1] lipid raft marker). F) Densitometric quantification of ALCAM and actin in the fraction three of the FC1199-OVA CLDN18^{+/+} and FC1199-OVA CLDN18/tumor cells. G) In vitro kinetics of CTL interactions (parameters: contact time in minutes, and arrest index) with FC1199-OVA CLDN18^{+/+} and CLDN18^{-/-} tumor cells treated with blocking ALCAM antibody (ALCAM) or isotype (Iso). H) ALCAM interference on FC1199-OVA CLDN18^{+/+} or FC1199-OVA CLDN18^{-/-} cell clones; membrane ALCAM FC quantification after cell transfection with ALCAM-targeting siRNA (siALCAM) or irrelevant (siSCR) siRNA (left panel) and in vitro analysis of the ability to support IS formation in CTLs (right panel). I) TIL quantification by FC in FC1199-OVA CLDN18^{+/+} and FC1199-OVA CLDN18^{-/-} tumor bearing mice receiving ALCAM blocking antibody (ALCAM) or isotype (Iso) before ACT.

FIG. 6 CLDN18 is prognostic biomarker of better OS in PDAC patients and supports cancer immune surveillance in immunogenic autochthonous model of lung adenocarcinoma. A) Representative spatial analysis of pancreatic tissues from patients diagnosed with early stages pancreatic tumors. Different pancreatic areas (Normal in green, PanIN1 in yellow, PanIN2 in cyan, PDAC in red) and cell phenotypes (CLDN18+ tumor cells, CD4+ and CD8+ T cells) were annotated by pathologist (left panel). Quantification of CLDN18+ tumor cells, CD3+ and CD8+ TILs in the different areas of pancreatic tissue (right panel). B) Kaplan-Meier curves showing the overall survival of 148 PDAC patients based on CLDN18 expression and TILs as single markers or in association. C) Kaplan-Meier curves showing the overall survival of PDAC patients (Bailey et al., (2016) Nature 531, 47-52) based on CLDN18 expression. D) Genomic analysis performed on available PDAC datasets (cBioPortal for Cancer Genomics—Memorial Sloan Kettering Cancer Center) revealing that CLDN18 is not target of mutation events during cancer progression (dataset: 1_TCGA, Nature 2020; 2_Dana Farber, Nat Genet 2018; 3_Umich, Nature 2017; 4_MSK, 5_Nat Genet 2020; 6_MSK, Clin Cancer Res 2020). E) Quantification of the number of T cells either close (distance<100 μm) or far (distance>100 μm) from CLDN18+ cells within the CLDN18+ PDAC cases. F) T cells distribution in term of percentage at different distances from CLDN18+ tumor cells. Statistic was calculated by the Shapiro Wilk function. T cells preferentially localize in proximity of CLDN18+ tumor cells in PDAC microenvironment. G) H/E representative pictures of a lung lobe isolated from CLDN18 proficient and CLDN18 deficient lung lesions, 18 weeks after nose instillation with CRE lentivirus (Scale bar: 1 mm). H) Comparison of G1 (left) and G2 (right) tumor lesions in conditional CLDN18+ and CLDN18—KP mice. I) Tumor growth curves in immunodeficient mice challenged with CLDN18-deficient FC1242-OVA tumor cells, in situ transduced with GFP-luciferase (Luc) as control virus, GFP-CLDN18 (CLDN18) expressing viral particles or saline (CTRL) and adoptively transferred with OVA-specific CD8+ CTLs (ACT).

FIG. 7 Gemcitabine and PMA induce CLDN18 expression in DANG cells. A) DANG were cultured for 48 hours with Gemcitabine (Gem), PMA, or left untreated. DANG-CLDN18 was used as positive control. Reference plot of CLDN18 expression. B) Mean fluorescence intensity in treated samples has been quantified and normalized on the untreated sample to obtain a fold change of expression.

FIG. 8 CLDN18 expression is prognostic biomarker and supports cancer immune surveillance in immunogenic autochthonous model of lung adenocarcinoma. A) H/E representative staining of the lung lobes isolated from CLDN18^{+/+} and CLDN18^{-/-} KP mouse lung lesions, 18 weeks after nose instillation with LentiCRE lentivirus (Scale bar 1 mm). B) Comparison of Grade 1 (left panel), Grade 2 (center panel) and Grade1+Grade2 (right panel) tumor lesions in CLDN18^{+/+} and CLDN18^{-/-} KP mouse lungs. Data are presented as mean±SEM. Mann-Whitney test, one-tailed. C) CD8+ T cell quantification by immunofluorescence (IF) in lung tissues from CLDN18^{+/+} and CLDN18^{-/-} KP mice. Data are presented as mean±SEM. Mann-Whitney test, one-tailed.

FIG. 9 CLDN18 favors ALCAM accrual on the cell membrane A) In vitro kinetics of CTL arrest index with CLDN18^{+/+} and CLDN18^{-/-} tumor cells treated with CD6 and ALCAM blocking antibodies, blocking CD6 antibody or control isotypes. Data are presented as mean±SEM. Kruskal-Wallis test. B) IFN-γ quantification in the supernatants of CTLs co-cultured with CLDN18^{+/+} and CLDN18^{-/- FC}1199-OVA tumor cells in presence or absence of CD6 blockade (CD6). Data are presented as mean±SD. C) Ex-vivo FC quantification of OVA-specific TILs following ACT in immunodeficient (Rag2^{-/-}) mice bearing either CLDN18^{+/+} or CLDN18^{-/-} FC1199-OVA tumors; different mouse groups received CD6 blocking antibody (CD6) or isotype control (ISO) before ACT and were euthanized 3 days later. Data are presented as mean±SEM. Unpaired, two-tailed, t test.

FIG. 10 FC1242-OVA and FC1199-OVA recapitulate cold and hot pancreatic adenocarcinoma tumors in orthotopical injection A) Tumor growth in immunocompetent (C57Bl/6) mice bearing orthotopic FC1199-OVA and FC1242-OVA (n=8/group) evaluated by ultrasound (US). Data are presented as mean±SEM. 2-way ANOVA. B) Comparison of pancreatic tumor weight at sacrifice in immunocompetent mice. Pancreata's weight from naive mice is reported as control. Data are presented as mean±SEM. Mann-Whitney test, two-tailed. C) Orthotopic tumor growth in immunodeficient (Rag2^{-/-}) mice bearing FC1199-OVA and FC1242-OVA (n≥7/group) evaluated by US. Data are presented as mean±SEM. 2-way ANOVA. D) Comparison of tumor weight in immunodeficient mice bearing FC1199-OVA or FC1242-OVA. Pancreata weight from naive mice is reported as control. Data are presented as mean±SEM. Mann-Whitney test, two-tailed.

FIG. 11 Pharmacological treatments inducing CLDN18 expression A) Percentage of CLDN18+ cells in DANG human pancreatic cancer cell lines following treatment with different inhibitors: PRI-724 (WNT/β Cathenin), TPCA-1 (NF-Kb), Verteporfin (Hippo), PMA, gemcitabine, CoCl2 and PX-478 (HIF inhibitors). DANG-CLDN18 cells were used as control (constitutively expressing the isoform predominant in pancreatic cancers, i.e. CLDN18.2). Unpaired, two-tailed, t test.

### DETAILED DESCRIPTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The practice of the present disclosure will employ, unless otherwise indicated, conventional chemistry, biochemistry, pharmaceutical, cell biology, immunology, and recombinant DNA techniques which are explained in the literature in the field.

Throughout this specification and the claims which follow, unless the context requires otherwise, the word “comprise”, and variations such as “comprises” and “comprising”, will be understood to imply the inclusion of a stated feature, element, member, integer or step or group of features, elements, members, integers or steps but not the exclusion of any other feature, element, member, integer or step or group of features, elements, members, integers or steps. The term “consisting essentially of” limits the scope of a claim or disclosure to the specified features, elements, members, integers, or steps and those that do not materially affect the basic and novel characteristic(s) of the claim or disclosure. The term “consisting of” limits the scope of a claim or disclosure to the specified features, elements, members, integers, or steps. The term “comprising” encompasses the term “consisting essentially of” which, in turn, encompasses the term “consisting of”. Thus, at each occurrence in the present disclosure, the term “comprising” may be replaced with the term “consisting essentially of” or “consisting of”. Likewise, at each occurrence in the present disclosure, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

The term “optional” or “optionally” as used herein means that the subsequently described event, circumstance or condition may or may not occur, and that the description includes instances where said event, circumstance, or condition occurs and instances in which it does not occur.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%.

In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In the following, definitions and embodiments will be provided which apply to all aspects of the present disclosure. Terms which are defined in the following have the meanings as defined unless otherwise indicated. Any undefined terms have their art recognized meanings.

### Certain definitions

Administering: As used herein, the term “administering” or “administration” typically refers to the administration of a composition to a subject to be treated to achieve delivery of an agent that is, or is included in, a composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Antigen: As used herein, an “antigen” covers any substance that will elicit an immune response and/or any substance against which an immune response or an immune mechanism such as a cellular response and/or humoral response is directed. This also includes situations wherein the antigen is processed into antigen peptides and an immune response or an immune mechanism is directed against one or more antigen peptides, in particular if presented in the context of MHC molecules. In particular, an “antigen” relates to any substance, such as a peptide or polypeptide, that reacts specifically with antibodies or T-lymphocytes (T-cells). The term “antigen” may comprise a molecule that comprises at least one epitope, such as a B cell epitope and/or T cell epitope. In some embodiments, an antigen is a molecule which, optionally after processing, induces an immune reaction, which may be specific for the antigen (including cells expressing the antigen). In some embodiments, an antigen is a disease-associated antigen, such as a tumor antigen, a viral antigen, or a bacterial antigen, or an epitope derived from such antigen. In some embodiments, an antigen is presented or present on the surface of cells of the immune system such as antigen presenting cells like dendritic cells or macrophages. An antigen or a procession product thereof such as a T cell epitope is in some embodiments bound by an antigen receptor such as a T cell receptor or antibody. Accordingly, an antigen or a procession product thereof may react specifically with immune effector cells such as T-lymphocytes (T cells).

Antibody: As used herein, the term “antibody” refers to an agent that specifically binds to a particular antigen. In some embodiments, the term encompasses any polypeptide or polypeptide complex that includes immunoglobulin structural elements sufficient to confer specific binding. Exemplary antibodies include, but are not limited to, monoclonal antibodies or polyclonal antibodies. In some embodiments, an antibody may include one or more constant region sequences that are characteristic of mouse, rabbit, primate, or human antibodies. In some embodiments, an antibody may include one or more sequence elements which are humanized, primatized, chimeric, etc., as is known in the art. In many embodiments, the term “antibody” is used to refer to one or more of the art-known or developed constructs or formats for utilizing antibody structural and functional features in alternative presentation. For example, in some embodiments, an antibody in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated complementarity determining regions (CDRs) or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, the term “antibody” refers to a glycoprotein comprising at least two heavy (H) chains and two light (L) chains inter-connected by disulfide bonds. In some embodiments, each heavy chain is comprised of a heavy chain variable region (VH) and a heavy chain constant region (CH). In some embodiments, each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3 (or CDR-H1, CDR-H2 and CDR-H3), the CDRs of a VL are termed LCDR1, LCDR2 and LCDR3 (or CDR-L1, CDR-L2 and CDR-L3). The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding to host tissues or factors, including various cells of the immune system (e.g., effector cells) and components of the complement system such as C1q. The term “full-length” when used in the context of an antibody indicates that the antibody is not a fragment, but contains all of the domains of the particular isotype normally found for that isotype in nature, e.g. the VH, CHI, CH2, CH3, hinge, VL and CL domains for an IgG1 antibody. As used herein, the term “Fab-arm” or “arm” refers to one heavy chain-light chain pair and is used interchangeably with “half molecule” herein. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.].

Antibodies can be made by the skilled person using methods and commercially available services and kits known in the art. For example, methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

An antibody may exert its therapeutic effect through recruiting the patient's immune system to destroy tumor cells and/or through a therapeutic moiety or agent coupled to the antibody. In some embodiments, an antibody is capable of acting through recruiting the patient's immune system to destroy tumor cells, i.e., the antibody, in particular when bound to its target such as a tumor antigen on a diseased cell, elicits immune effector functions as described herein. Preferably, said immune effector functions are directed against cells such as cancer cells carrying a tumor antigen on their surface.

Associated with: Two events or entities are “associated” with one another, as that term is used herein, if the presence, level and/or form of one is correlated with that of the other.

Cancer: The term “cancer” is used herein to generally refer to a disease or condition in which cells of a tissue of interest exhibit relatively abnormal, uncontrolled, and/or autonomous growth, so that they exhibit an aberrant growth phenotype characterized by a significant loss of control of cell proliferation. In some embodiments, cancer may comprise cells that are precancerous (e.g., benign), malignant, pre-metastatic, metastatic, and/or non-metastatic. In some embodiments, cancer may be characterized by a solid tumor. In some embodiments, cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, ovarian cancer, breast cancer, glioblastomas, colorectal cancer, gastro-intestinal cancers and nervous system cancers, benign lesions such as papillomas, and the like. The term “cancer” also includes “metastasis” of cancer. In some embodiments, a cancer involves cancer cells expressing a tumor antigen. In some embodiments, expression of the tumor antigen is at the surface of the cells and/or the tumor antigen is presented at the surface of the cells in the context of MHC molecules. In some embodiments, at least 50%, 60%, 70%, 80% or 90% of the cancer cells express a tumor antigen.

CLDN18 positive: As used herein, the term “CLDN18 positive” or “CLDN18+” refers to clinically relevant CLDN18 expression and/or activity, e.g., as may be associated with a particular disease, disorder, or condition and/or as may be detected in or on a sample that may be or comprise one or more cells or tissue samples. In some embodiments, CLDN18+refers to cancer that is associated with clinically relevant CLDN18 expression and/activity. In certain exemplary embodiments, CLDN18 positive expression and/or activity may be or comprise de novo CLDN18 overexpression, e.g., in cancer cells; alternatively or additionally, in some embodiments, CLDN18 positive expression and/or activity may be or have been associated with exposure to one or more agents or conditions, such as one or more chemotherapeutic agents (including, e.g., gemcitabine and/or cisplatin). In some embodiments, CLDN18 “positivity” is assessed relative to an appropriate reference (e.g., a “negative control” such as a CLDN18 level and/or activity in appropriately comparable non-cancer cell(s) and/or tissue(s); a “positive control” such as a CLDN18 level and/or activity as may have been determined for known CLDN18-positive cell(s) and/or tissue(s); and/or an established threshold for CLDN18 level and/or activity associated with normal (e.g., healthy, non-cancer) vs non-normal (e.g., cancer) status. In some embodiments, the term “CLDN18+” is used herein to refer to a tumor sample from a cancer patient when that has been determined to show elevated detectable CLDN18 protein expression relative to an appropriate reference (e.g., that level observed in a sample determined or otherwise known to be negative for CLDN18 expression).

Clinical outcome: As used herein, the term “clinical outcome” refers to the clinical result of a disease, e.g. reduction or amelioration of symptoms, in particular following a treatment.

Co-administration: As used herein, the term “co-administration” refers to use of a therapy in combination with another therapy, so that a subject receives both. The combined administration of therapies may be performed concurrently (e.g., via overlapping protocols) or separately (e.g., sequentially in any order). In some embodiments, a pharmaceutical composition may include two or more active agents combined in one pharmaceutically-acceptable carrier (e.g., in a single dosage form). Alternatively, in some embodiments, co-administration involves administration of two or more physically distinct pharmaceutical compositions, each of which may contain a different active agent or combination of agents; in some such embodiments, one or more (and, in some embodiments, all) doses of such distinct pharmaceutical compositions may be administered substantially simultaneously. In some embodiments, one or more (and, in some embodiments, all) doses of such distinct pharmaceutical compositions may be administered separately, e.g., according to overlapping regimens or sequential regimens. In general, two or more therapies may be considered to be “co-administered” when delivered or administered sufficiently close in time that there is at least some temporal overlap in biological effect(s) generated by each on a target cell or a subject to which they are administered.

Combination therapy: As used herein, the term “combination therapy” refers to those situations in which a subject is simultaneously exposed to two or more therapeutic regimens (e.g., two or more therapeutic agents). In some embodiments, two or more regimens may be administered simultaneously; in some embodiments, such regimens may be administered sequentially (e.g., all doses of a first regimen are administered prior to administration of any doses of a second regimen); in some embodiments, such agents are administered in overlapping dosing regimens. In some embodiments, administration of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Determining: The term “determining” is used herein in a broad sense to include appropriate means for detecting the presence or absence of a condition, situation, or entity of interest or any form of measurement of a condition, situation, or entity of interest in a sample.

Quantitative and qualitative determinations, measurements, or assessments are included, including semi-quantitative. Such determinations, measurements, or assessments may be relative, such as when an entity of interest is detected relative to a control reference, or absolute. Therefore, the term “quantification,” when used in the context of quantifying an entity of interest, may refer to absolute or relative quantification.

Disease: As used herein, the term “disease” refers to a disorder or condition that typically impairs normal functioning of a tissue or system in a subject (e.g., a human subject) and is typically manifested by characteristic signs and/or symptoms. In some embodiments, an exemplary disease is cancer.

Epitope: As used herein, the term “epitope” refers to an antigenic determinant in a molecule such as an antigen, i.e., to a part in or fragment of the molecule, that is recognized by the immune system, for example, that is recognized by antibodies, T cells or B cells, in particular when presented in the context of MHC molecules. An epitope of a protein may comprises a continuous or discontinuous portion of said protein and, e.g., may be between about 5 and about 100, between about 5 and about 50, between about 8 and about 30, or about 10 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, the epitope in the context of the present disclosure is a T cell epitope which refers to a part or fragment of a protein that is recognized by a T cell when presented in the context of MHC molecules.

Expression level: As used herein, the term “expression level” may refer to the amount of protein and/or mRNA, preferably protein, of a certain target gene such as CLDN18 in a sample. In some embodiments, the term “low expression level” refers to the fact that protein and/or mRNA, preferably protein is not or essentially not present in a sample and preferably is not present in a readily detectable amount in a sample and/or that the protein and/or mRNA, preferably protein is present in a lower amount compared to a reference. In some embodiments, the term “high expression level” refers to the fact that the protein and/or mRNA, preferably protein is present in a sample preferably in a readily detectable amount and/or that the protein and/or mRNA, preferably protein is present in a higher amount compared to a reference. In some embodiments, the term “expression level” may refer to the number of cells expressing protein and/or mRNA, preferably protein, of a certain target gene such as CLDN18 in a sample. For example, the term “high expression level of CLDN18” includes a percentage of CLDN18-positive cells of greater than 5%, greater than 10%, greater than 15%, greater than 20%, greater than 25%, greater than 30%, greater than 35%, greater than 40%, greater than 45%, greater than 50%, greater than 55%, greater than 60%, or even greater. For example, the term “low expression level of CLDN18” includes a percentage of CLDN18-positive cells of lower than 5%, lower than 4%, lower than 3%, lower than 2%, lower than 1%, or even lower.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “substantially identical” to one another if their sequences are at least 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Immune effector functions: As used herein, the term “immune effector functions” includes any functions mediated by components of the immune system that result e.g. in the inhibition of tumor growth and/or inhibition of tumor development, including inhibition of tumor dissemination and metastasis. Preferably, immune effector functions result in killing of cancer cells. Such functions comprise complement dependent cytotoxicity (CDC), antibody-dependent cell-mediated cytotoxicity (ADCC), antibody-dependent cell-mediated phagocytosis (ADCP), induction of apoptosis, cytolysis, and/or inhibition of proliferation of cells.

ADCC describes the cell-killing ability of effector cells, in particular lymphocytes, which preferably requires the target cell being marked by an antibody. ADCC preferably occurs when antibodies bind to antigens on tumor cells and the antibody Fc domains engage Fc receptors (FcR) on the surface of immune effector cells. Several families of Fe receptors have been identified, and specific cell populations characteristically express defined Fe receptors.

ADCC can be viewed as a mechanism to directly induce a variable degree of immediate tumor destruction that leads to antigen presentation and the induction of tumor-directed T-cell responses. Preferably, in vivo induction of ADCC will lead to tumor-directed T-cell responses and host-derived antibody responses.

CDC is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the CH2 domains of participating antibody molecules such as IgG molecules (C1q is one of three subcomponents of complement Cl). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell.

Immune response: As used herein, the term “immune response” refers to an integrated bodily response, generally to an antigen or cells expressing an antigen, and may refer to a cellular immune response, a humoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, such as CD4+ and CD8+ T-lymphocytes, e.g. CD8⁺ T-lymphocytes.

The terms “inducing an immune response” and “eliciting an immune response” and similar terms in the context of the present disclosure refer to the induction of an immune response, such as the induction of a cellular immune response, a humoral immune response, or both. “Inducing” in this context may mean that there was no immune response before induction, but it may also mean that there was a certain level of immune response before induction and after induction said immune response is enhanced. Thus, “inducing an immune response” in this context also includes “enhancing an immune response”. In some embodiments, after inducing an immune response in an individual, said individual is protected from developing a disease such as a cancerous disease or the disease condition is ameliorated by inducing an immune response.

Immunogenicity: “Immunogenicity” is the ability of a substance to provoke an immune response in the body of a human or other animal. The innate immune system is the component of the immune system that is relatively unspecific and immediate. It is one of two main components of the vertebrate immune system, along with the adaptive immune system.

Immunoglobulin: As used herein, the term “immunoglobulin” relates to proteins of the immunoglobulin superfamily, preferably to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies. The structure of immunoglobulins has been well characterized. See, e.g., Fundamental Immunology Ch. 7 (Paul, W., ed., 2^{nd} ed. Raven Press, N.Y. (1989)). Briefly, immunoglobulins generally comprise several chains, typically two identical heavy chains and two identical light chains which are linked via disulfide bonds. These chains are primarily composed of immunoglobulin domains or regions, such as the VL or VL (variable light chain) domain/region, C_{L} or CL (constant light chain) domain/region, VH or VH (variable heavy chain) domain/region, and the C_{H} or CH (constant heavy chain) domains/regions C_{H}1 (CHI), C_{H}2 (CH2), C_{H}3 (CH3), and C_{H}4 (CH4). The heavy chain constant region typically is comprised of three domains, CH1, CH2, and CH3. The hinge region is the region between the CHI and CH2 domains of the heavy chain and is highly flexible. Disulfide bonds in the hinge region are part of the interactions between two heavy chains in an IgG molecule. Each light chain typically is comprised of a VL and a CL. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

Increase: Terms such as “increase” or “enhance” or as used herein means the ability to cause an overall increase, or enhancement, for example, by at least about 5% or greater, about 10% or greater, about 15% or greater, about 20% or greater, about 25% or greater, about 30% or greater, about 40% or greater, about 50% or greater, about 75% or greater, or about 100% or greater in the level.

Inhibit: Terms such as “inhibit” or “reduce” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e., a reduction to zero or essentially to zero.

Indicate: As used herein, the term “indicate” refers to some degree of probability or likelihood. If a first event “indicates” a second event, e.g., that a patient is responding to immunotherapy, the occurrence of the first event means that it is likely that the second event will occur, and optionally, it is more likely that the second event will occur than that the second event will not occur.

Isolated: “Isolated” means removed (e.g., purified) from the natural state or from an artificial composition, such as a composition from a production process. For example, a nucleic acid, peptide or polypeptide naturally present in a living animal is not “isolated”, but the same nucleic acid, peptide or polypeptide partially or completely separated from the coexisting materials of its natural state is “isolated”. An isolated nucleic acid, peptide or polypeptide can exist in substantially purified form, or can exist in a non-native environment such as, for example, a host cell.

Locally advanced tumor: As used herein, the term “locally advanced tumor” or “locally advanced cancer” refers to its art-recognized meaning, which may vary with different types of cancer. For example, in some embodiments, a locally advanced tumor refers to a tumor that is large but has not yet spread to another body part. In some embodiments, a locally advanced tumor is used to describe cancer that has grown outside the tissue or organ it started but has not yet spread to distant sites in the body of a subject. By way of example only, in some embodiments, locally advanced pancreatic cancer typically refers to stage III disease with tumor extension to adjacent organs (e.g., lymph nodes, liver, duodenum, superior mesenteric artery, and/or celiac trunk) but no signs of metastatic disease; yet complete surgical excision with negative pathologic margins is not possible.

Metastasis: As used herein, the term “metastasis” means the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs.

Finally, the growth of a new tumor at the target site depends on angiogenesis. Tumor metastasis often occurs even after the removal of the primary tumor because tumor cells or components may remain and develop metastatic potential. In some embodiments, the term “metastasis” relates to “distant metastasis” which relates to a metastasis which is remote from the primary tumor and the regional lymph node system. In some embodiments, the term “metastasis” relates to lymph node metastasis.

Nucleic acid/Polynucleotide: As used herein, the term “nucleic acid” refers to a polymer of nucleotides, e.g., at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis). In some embodiments, a nucleic acid is at least 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000,1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Patient: As used herein, the term “patient” refers to any organism who is suffering or at risk of a disease or disorder or condition. Typical patients include animals (e.g., mammals such as mice, rats, rabbits, non-human primates, and/or humans). In some embodiments, a patient is a human. In some embodiments, a patient is suffering from or susceptible to one or more diseases or disorders or conditions. In some embodiments, a patient displays one or more symptoms of a disease or disorder or condition. In some embodiments, a patient has been diagnosed with one or more diseases or disorders or conditions. In some embodiments, a disease or disorder or condition that is amenable to provided technologies is or includes cancer, or presence of one or more tumors. In some embodiments, a patient is receiving or has received certain therapy to diagnose and/or to treat a disease, disorder, or condition. In some embodiments, a patient is a cancer patient.

Polypeptide: The term “polypeptide”, as used herein, typically has its art-recognized meaning of a polymer of amino acids, e.g., at least three amino acids or more. Those of ordinary skill in the art will appreciate that the term “polypeptide” is intended to be sufficiently general as to encompass not only polypeptides having a complete sequence, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Prognosis: The term “prognosis”, as used herein, refers to a prediction of outcome, e.g., the probability of overall, disease free, progression-free survival (PFS) or disease-free survival (DFS). Survival is usually calculated as an average number of months (or years) that 50% of patients survive, or the percentage of patients that are alive, e.g., after 1, 5, 15, or 20 years. Prognosis is important for treatment decisions because patients with a good prognosis are usually offered less invasive treatments, while patients with poor prognosis are usually offered more aggressive treatments, such as more extensive chemotherapy drugs.

As used herein, the term “poor prognosis” refers to a decreased patient survival and/or an early disease progression and/or an increased disease recurrence and/or an increased metastasis formation. As used herein, the term “good prognosis” refers to an increased patient survival and/or no or a late disease progression and/or no or a decreased disease recurrence and/or no or a decreased metastasis formation.

Prognostic marker: The term “prognostic marker”, as used herein, refers to a biomarker, e.g., a compound, e.g., CLDN18, used to predict or monitor clinical outcome of a subject affected with a disease, e.g., cancer.

Prediction: The term “prediction”, as used herein, refers to providing information about the possible response or outcome.

Recombinant: The term “recombinant”, as used herein, means “made through genetic engineering”. In some embodiments, a “recombinant object” in the context of the present disclosure is not occurring naturally.

Reference/Reference standard: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. In some embodiments, a reference or control is or comprises a set specification (e.g., relevant acceptance criteria). Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

“Respond”: The term “respond” or similar terms such as “responsive”, as used herein, refer, in a therapeutic setting, to the fact that a patient has a therapeutic benefit from a given mode of treatment and, in particular, to the observation of an alleviation, prevention or elimination of a disease including shortening the duration of a disease, arresting or slowing progression or worsening of a disease, inhibiting or slowing the development of a new disease and/or recurrences, preventing or delaying the onset of a disease or the symptoms thereof, decreasing the frequency or severity of symptoms in a patient who currently has or who previously has had a disease and/or prolonging the lifespan of the patient. In particular, they refer to the observation of a reduction in tumor mass or of an increase in tumor free time, recurrence free time or overall survival time. Terms such as “non-responsive” or “non-responder” refer, in a therapeutic setting, to the fact that a patient has no therapeutic benefit from a given mode of treatment and, in particular, to no observation of an alleviation, prevention or elimination of a disease, i.e. the patient is resistant to treatment.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods).

Sample: The term “sample”, as used herein, refers to any material which is obtained from a subject and which may be used for analytical purposes, in particular in the methods described herein. In certain embodiments, a sample can be or can be derived from any tissues, cells and/or cells in biological fluids from, for example, a mammal or human to be tested. A sample may be isolated from a patient, e.g. from the human body. A sample can be a fractionated and/or purified sample. The sample may be obtained from a patient prior to initiation of a therapeutic treatment, during the therapeutic treatment, and/or after the therapeutic treatment, e.g. prior to, during or following the administration of cancer therapy. In some embodiments, the term “sample”, as used herein, means any sample containing cells derived from a subject.

Examples of samples include fluids such as blood, plasma, saliva, urine and seminal fluid samples as well as biopsies, organs, tissues or cell samples. The sample may be treated prior to its use. The term “sample” includes a “cancer sample” which refers to any sample containing tumoral cells derived from a patient. Preferably, the sample contains only tumoral cells. The term “normal sample” refers to any sample which does not contain any tumoral cells. The methods of the invention as disclosed herein, may be in vivo, ex vivo or in vitro methods, preferably in vitro methods.

Selective or specific: The term “selective” or “specific”, when used herein in reference to an agent, is understood by those skilled in the art to mean that the agent discriminates between potential target entities, states, or cells. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of a target-binding moiety for one or more other potential target entities (e.g., competitors). In some embodiments, specificity is evaluated relative to that of a reference specific binding moiety. In some embodiments, specificity is evaluated relative to that of a reference non-specific binding moiety.

Subject: As used herein, the term “subject” refers to human beings, non-human primates or other mammals (e.g. mouse, rat, rabbit, dog, cat, cattle, swine, sheep, horse or primate) that can be afflicted with or are susceptible to a disease or disorder (e.g., cancer) but may or may not have the disease or disorder. In some embodiments, a subject is a human subject. In some embodiments, a subject is suffering from a disease, disorder, or condition (e.g., cancer). In some embodiments, a subject is susceptible to a disease, disorder, or condition (e.g., cancer). In some embodiments, a subject displays one or more symptoms or characteristics of a disease, disorder, or condition (e.g., cancer). In some embodiments, a subject is an individual to whom diagnosis and/or therapy is and/or has been administered. Unless otherwise stated, the term “subject” do not denote a particular age, and thus encompass adults, elderlies, children, and newborns. In preferred embodiments, the “subject” is a “patient”. The term “patient” means a subject for treatment, in particular a diseased subject.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from the disease, disorder, or condition; carrier of a genetic marker or other biomarker associated with the disease, disorder or condition, etc.).

Suffering from: An individual who is “suffering from” a disease, disorder, and/or condition has been diagnosed with and/or displays one or more symptoms of a disease, disorder, and/or condition.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

T cell: The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen.

Therapy: As used herein, the term “therapy” refers to an intervention that, when administered to a subject or a patient, has a therapeutic effect and/or elicits a desired biological and/or pharmacological effect. In some embodiments, a therapy is any substance that can be used to alleviate, ameliorate, relieve, inhibit, prevent, delay onset of, reduce severity of, and/or reduce incidence of one or more symptoms or features of a disease, disorder, and/or condition. In some embodiments, a therapy is a medical intervention (e.g., surgery, radiation, phototherapy) that can be performed to alleviate, relieve, inhibit, present, delay onset of, reduce severity of, and/or reduce incidence of one or more symptoms or features of a disease, disorder, and/or condition. As used herein, the term “therapy” includes any type of treatment of cancer (i.e., antitumoral therapy), including an adjuvant therapy and a neoadjuvant therapy. Therapy comprises radiotherapy and therapies, preferably systemic therapies such as hormone therapy, chemotherapy, immunotherapy and monoclonal antibody therapy.

The term “adjuvant therapy”, as used herein, refers to any type of treatment of cancer given as additional treatment, usually after surgical resection of the primary tumor, in a patient affected with a cancer that is at risk of metastasizing and/or likely to recur. The aim of such an adjuvant treatment is to improve the prognosis. Adjuvant therapies comprise radiotherapy and therapy, preferably systemic therapy, such as hormone therapy, chemotherapy, immunotherapy and monoclonal antibody therapy.

The term “neoadjuvant therapy”, as used herein, refers to any type of treatment of cancer given prior to surgical resection of the primary tumor, in a patient affected with a cancer. The most common reason for neoadjuvant therapy is to reduce the size of the tumor so as to facilitate a more effective surgery. Neoadjuvant therapies comprise radiotherapy and therapy, preferably systemic therapy, such as hormone therapy, chemotherapy, immunotherapy and monoclonal antibody therapy.

As used herein, the term “chemotherapeutic treatment” or “chemotherapy” refers to a cancer therapeutic treatment using chemical or biochemical substances, in particular using one or several antineoplastic agents.

The term “radiotherapeutic treatment” or “radiotherapy” is a term commonly used in the art to refer to multiple types of radiation therapy including internal and external radiation therapies or radio immunotherapy, and the use of various types of radiations including X-rays, gamma rays, alpha particles, beta particles, photons, electrons, neutrons, radioisotopes, and other forms of ionizing radiations.

The term “immunotherapy” refers to a cancer therapeutic treatment using the immune system to reject cancer. The therapeutic treatment stimulates the patient's immune system to attack the malignant tumor cells. It includes immunization of the patient with tumoral antigens (eg. by administering a cancer vaccine), in which case the patient's own immune system is trained to recognize tumor cells as targets to be destroyed, or administration of molecules stimulating the immune system such as cytokines, or administration of therapeutic antibodies as drugs, in which case the patient's immune system is recruited to destroy tumor cells by the therapeutic antibodies. In particular, antibodies are directed against specific antigens such as the unusual antigens that are presented on the surfaces of tumors. As illustrating example, one can cite Trastuzumab or Herceptin antibody which is directed against HER2 and approved by FDA for treating breast cancer.

The term “monoclonal antibody therapy” refers to any antibody that functions to deplete tumor cells in a patient. In particular, therapeutic antibodies specifically bind to antigens present on the surface of the tumor cells, e.g. tumor specific antigens present predominantly or exclusively on tumor cells. Alternatively, therapeutic antibodies may also prevent tumor growth by blocking specific cell receptors.

The term “hormone therapy” or “hormonal therapy” refers to a cancer treatment having for purpose to block, add or remove hormones. For instance, in breast cancer, the female hormones estrogen and progesterone can promote the growth of some breast cancer cells. So in these patients, hormone therapy is given to block estrogen and a non-exhaustive list commonly used drugs includes: Tamoxifen, Fareston, Arimidex, Aromasin, Femara, Zoladex/Lupron, Megace, and Halotestin.

Threshold level (reference level): As used herein, the term “threshold level” refers to a level that is used as a reference to attain information on and/or classify the results of a measurement, for example, the results of a measurement attained in an assay. For example, in some embodiments, a threshold level means a value measured in an assay that defines the dividing line between two subsets of a population. Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Transfection: As used herein, the term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient, or the cell may be in vitro, e.g., outside of a patient. Thus, according to the present disclosure, a cell for transfection of a nucleic acid described herein can be present in vitro or in vivo, e.g. the cell can form part of an organ, a tissue and/or the body of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection, for example. RNA can be transfected into cells to transiently express its coded protein.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition. In some embodiments, treatment may be administered to a subject at a later-stage of disease, disorder, and/or condition. A treatment of cancer may eliminate cancer, reduce the size or the number of tumors in a patient, arrest or slow the development of cancer in a patient, inhibit or slow the development of new cancer in a patient, decrease the frequency or severity of symptoms in a patient, and/or decrease recurrences in a patient who currently has or who previously has had cancer.

Tumor: As used herein, the term “tumor” or “tumor disease” refers to an abnormal growth of cells (called neoplastic cells, tumorigenous cells or tumor cells) preferably forming a swelling or lesion. By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

Tumor antigen: As used herein, the term “tumor antigen” or “tumor-associated antigen” relates to an antigen which is present in tumor cells. Preferably the antigen is present on tumor cells, such as on the surface of tumor cells. Preferably, the “tumor antigen” is expressed by tumor cells. In one embodiment, the term “tumor antigen” relates to proteins which are aberrantly expressed in tumor cells when compared to the normal, i.e. non-tumorous, cells.

For example, expression may be only found in tumor cells but not in the normal, i.e. non-tumorous, cells or the level of expression may be higher in tumor cells compared to the normal, i.e. non-tumorous, cells. In one embodiment, the term “tumor antigen” relates to proteins that are under normal conditions specifically expressed in a limited number of tissues and/or organs or in specific developmental stages and are expressed or aberrantly expressed in one or more tumor or cancer tissues. In some embodiments, a tumor antigen is preferably associated with the cell surface of a cancer cell and is preferably not, only rarely or at a lower level expressed in normal tissues and cells. Preferably, a tumor antigen is not expressed in a cell if the level of expression is below the detection limit and/or if the level of expression is too low to allow binding by tumor antigen-specific antibodies added to the cells.

Unresectable tumor: As used herein, the term “unresectable tumor” typically refers to a tumor characterized by one or more features that, in accordance with sound medical judgement, are considered to indicate that the tumor cannot safely (e.g., without undue harm to the subject) be removed by surgery, and/or with respect to which a competent medical profession has determined that risk to the subject of tumor removal outweighs benefits associated with such removal. In some embodiments, an unresectable tumor refers to a tumor that involves and/or has grown into an essential organ or tissue (including blood vessels that may not be reconstructable) and/or that is otherwise in a location that cannot readily be surgically accessed without unreasonable risk of damage to one or more other critical or essential organs and/or tissues (including blood vessels). In some embodiments, “unresectability” of a tumor refers to the likelihood of achieving a margin-negative (RO) resection. In the context of pancreatic cancer, encasement of major vessels by a tumor such as superior mesenteric artery (SMA) or celiac axis, portal vein occlusion, and the presence of celiac or para-aortic lymphadenopathy are generally acknowledged as findings that preclude RO surgery. Those skilled in the art will understand parameters that determine whether a tumor is unresectable or not.

Variant: As used herein, the term “variant” with reference to an amino acid sequence (peptide or polypeptide) means an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, such as from 1 to about 10 or from 1 to about 5 amino acid differences compared to the parent.

For the purposes of the present disclosure, “variants” of an amino acid sequence (peptide or polypeptide) may comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

In some embodiments, the degree of similarity, such as identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. In some embodiments, the degree of similarity or identity is given for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given, e.g., for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, such as sequence identity can be done with art known tools, such as using the best sequence alignment, for example, using Align, using standard settings, preferably EMBOSS::needle, Matrix: Blosum62, Gap Open 10.0, Gap Extend 0.5.

### Certain Embodiments of the Invention

In one aspect, the present invention provides methods for predicting clinical outcome of a subject affected with cancer, wherein the method comprises the step of determining the expression level of CLDN18 in a sample, e.g., a cancer sample, from said subject. A high expression level of CLDN18 is indicative of a good prognosis. A low expression level of CLDN18 is indicative of a poor prognosis. A low expression level of CLDN18 may indicate a decreased patient survival and/or an early disease progression and/or an increased disease recurrence and/or an increased metastasis formation. A high expression level of CLDN18 may indicate an increased patient survival and/or no or late disease progression and/or no or a decreased disease recurrence and/or no or a decreased metastasis formation. In some embodiments, the expression level of CLDN18 is a prognosis marker for clinical outcome without any therapeutic intervention being applied. In some embodiments, the expression level of CLDN18 is a prognosis marker for clinical outcome with therapeutic intervention being applied, e.g., immunotherapy being administered. In addition, CLDN18 is a prognostic marker that can be used for all types of cancers, in particular pancreatic cancer.

In a further aspect, the present invention provides methods for selecting a subject affected with cancer for immunotherapy, e.g., given as an adjuvant therapy, or determining whether a subject affected with cancer is susceptible to benefit from immunotherapy, e.g., given as an adjuvant therapy, wherein the method comprises the step of determining the expression level of CLDN18 in a sample, e.g., a cancer sample, from said subject. A high expression level of CLDN18 indicates that immunotherapy is required or indicated. A low expression level of CLDN18 indicates that immunotherapy is not required or not indicated.

In a further aspect, the present invention provides methods for improving responsiveness of a subject affected with cancer to immunotherapy, e.g., given as an adjuvant therapy, or improving the possibility of a subject affected with a cancer to benefit from immunotherapy, e.g., given as an adjuvant therapy. The method comprises administering an agent stabilizing or increasing expression of CLDN18 to the subject.

In some embodiments of these above mentioned methods, a method further comprises the step of providing a sample, e.g., a cancer sample, from the subject.

The expression level of CLDN18 can be determined from a sample, e.g., a cancer sample, by a variety of techniques.

In some embodiments, a determination of the expression level of CLDN18 comprises a quantitative determination of the expression level of CLDN18. In some embodiments, the expression level of CLDN18 is determined by measuring the quantity of CLDN18 protein or CLDN18 mRNA.

In some embodiments of these above mentioned methods, the expression level of CLDN18 is determined by measuring the quantity of CLDN18 protein. The quantity of CLDN18 protein may be measured by any methods known by the skilled person, such as by immunofluorescence or immunohistochemistry applications. Usually, these methods comprise contacting the sample with a binding partner capable of selectively interacting with the CLDN18 protein present in the sample. The binding partner is generally a polyclonal or monoclonal antibody, preferably monoclonal. In some embodiments, the antibody is specific to CLDN18.1 compared to CLDN18.2, i.e. the antibody specific to CLDN18.1 does not cross-react with CLDN18.2. In some embodiments, the antibody is specific to CLDN18.2 compared to CLDN18.1, i.e. the antibody specific to CLDN18.2 does not cross-react with CLDN18.1. In some embodiments, the antibody is specific to CLDN18.1 and CLDN18.2, i.e. the antibody cross-reacts with CLDN18.1 and CLDN18.2. An antibody specific for CLDN18.1 compared to CLDN18.2 or specific for CLDN18.2 compared to CLDN18.1 may be prepared by using a part of the respective protein where the sequence identity is lower. The quantity of CLDN18 protein may be measured by semi-quantitative Western blots, enzyme-labeled and mediated immunoassays, such as ELISAs, biotin/avidin type assays, radioimmunoassay, Immunoelectrophoresis or immunoprecipitation or by protein or antibody arrays. The protein expression level may be assessed by immunohistochemistry on a tissue section of a cancer sample (e.g. frozen or formalin-fixed paraffin embedded material). The reactions generally include revealing labels such as fluorescent, chemiluminescent, radioactive, enzymatic labels or dye molecules, or other methods for detecting the formation of a complex between the antigen and the antibody or antibodies reacted therewith. Preferably, the quantity of CLDN18 protein is measured by immunohistochemistry or semi-quantitative Western blot.

In some embodiments of these above mentioned methods, the expression level of CLDN18 is determined by measuring the quantity of CLDN18 mRNA. Methods for determining the quantity of mRNA are well known in the art. For example, the nucleic acid contained in the sample (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR).

Preferably quantitative or semi-quantitative RT-PCR is preferred. Real-time quantitative or semi-quantitative RT-PCR is particularly advantageous. Primers may be easily designed by the skilled person. Other methods of Amplification include ligase chain reaction (LCR), transcription-mediated amplification (TMA), strand displacement amplification (SDA) and nucleic acid sequence based amplification (NASBA). Preferably, the quantity of CLDN18 mRNA is measured by quantitative or semi-quantitative RT-PCR or by real-time quantitative or semi-quantitative RT-PCR or by transcriptome approaches.

In some embodiments of these above mentioned methods, a method further comprises the step of comparing the expression level of CLDN18 to a reference expression level.

In particular, the reference expression level can be the expression level of CLDN18 in a normal sample. The normal sample is a non-tumoral sample, preferably from the same tissue as a cancer sample. The normal sample may be obtained from the subject affected with cancer or from another subject, preferably a normal or healthy subject, i.e. a subject who does not suffer from cancer. Preferably, the normal sample is obtained from the same subject as the cancer sample. Expression levels obtained from cancer and normal samples may be normalized by using expression levels of proteins which are known to have stable expression such as RPLPO (acidic ribosomal phosphoprotein PO), TBP (TATA box binding protein), GAPDH (glyceraldehyde 3-phosphate dehydrogenase) or β-actin.

Alternatively, the reference expression level may be the expression level of a gene having a stable expression in different cancer samples. Such genes include for example, RPLPO, TBP, GAPDH or β-actin.

In some embodiments, the reference expression level is the expression level of CLDN18 in one or more samples obtained from one or more subjects (i) having an ability to mount a natural or immunotherapy-induced immune response against cancer, (ii) having a favourable survival perspective, (iii) having an ability to respond to immunotherapy, or (iv) any combination of (i), (ii) and (iii). A level of CLDN18 at or above the reference level indicates that a subject affected with cancer (i) has the ability to mount a natural or immunotherapy-induced immune response against the cancer, (ii) has a good survival perspective, (iii) has the ability to respond to immunotherapy, or (iv) any combination of (i), (ii) and (iii).

In some embodiments, the reference expression level is the expression level of CLDN18 in one or more samples obtained from one or more subjects (i) not having an ability to mount a natural or immunotherapy-induced immune response against cancer, (ii) not having a favourable survival perspective, (iii) not having an ability to respond to immunotherapy, or (iv) any combination of (i), (ii) and (iii). A level of CLDN18 above the reference level indicates that a subject affected with cancer (i) has the ability to mount a natural or immunotherapy-induced immune response against the cancer, (ii) has a good survival perspective, (iii) has the ability to respond to immunotherapy, or (iv) any combination of (i), (ii) and (iii).

In some embodiments, a determination of the expression level of CLDN18 does not comprise a quantitative determination of the expression level of CLDN18. In some embodiments, a determination of the expression level of CLDN18 comprises a determination of the presence or absence of CLDN18. In some embodiments, the presence or absence of CLDN18 is determined by measuring the presence or absence of CLDN18 protein or CLDN18 mRNA. The presence or absence of CLDN18 protein may be measured by any methods known by the skilled person, such as by methods described above for measuring the quantity of CLDN18 protein. The presence or absence of CLDN18 mRNA may be measured by any methods known by the skilled person, such as by methods described above for measuring the quantity of CLDN18 mRNA.

In some embodiments, the presence of CLDN18 indicates that a subject affected with cancer (i) has the ability to mount a natural or immunotherapy-induced immune response against the cancer, (ii) has a good survival perspective, (iii) has the ability to respond to immunotherapy, or (iv) any combination of (i), (ii) and (iii). In some embodiments, the absence of CLDN18 indicates that a subject affected with cancer (i) has no or a poor ability to mount a natural or immunotherapy-induced immune response against the cancer, (ii) has a poor survival perspective, (iii) has no or a poor ability to respond to immunotherapy, or (iv) any combination of (i), (ii) and (iii).

In another aspect, the present invention further concerns a kit, and its use,
(a) for predicting clinical outcome of a subject affected with cancer and/or
(b) for selecting a subject affected with cancer for immunotherapy, or determining whether a subject affected with cancer is susceptible to benefit from immunotherapy, wherein the kit comprises:
(i) at least one antibody specific to CLDN18 and, optionally, means for detecting the formation of a complex between CLDN18 and said at least one antibody; and/or
(ii) at least one probe specific to CLDN18 mRNA or cDNA and, optionally, means for detecting the hybridization of said at least one probe on CLDN18 mRNA or cDNA; and/or
(iii) at least one nucleic acid primer pair specific to CLDN18 mRNA or cDNA and, optionally, means for amplifying and/or detecting said mRNA or cDNA, and,
(iv) optionally, a leaflet providing guidelines to use such a kit.

The inventors have also shown that increasing CLDN18 expression supports the efficacy of immunotherapy.

The present invention also provides methods for increasing the efficacy of immunotherapy comprising administering an agent stabilizing or increasing expression of CLDN18.

In this aspect, the present invention relates to a pharmaceutical composition or kit comprising:
(i) an agent stabilizing or increasing expression of CLDN18, and
(ii) an immunotherapeutic agent.

In some embodiments, an immunotherapeutic agent does not target CLDN18.2. In some embodiments, an immunotherapeutic agent targets an antigen other than CLDN18.2. In some embodiments, an immunotherapeutic agent does not target CLDN18.1. In some embodiments, an immunotherapeutic agent targets an antigen other than CLDN18.1. In some embodiments, an immunotherapeutic agent does not target CLDN18. In some embodiments, an immunotherapeutic agent targets an antigen other than CLDN18. In some embodiments, an immunotherapeutic agent does not target a claudin. In some embodiments, an immunotherapeutic agent targets an antigen other than a claudin. In some embodiments, an immunotherapeutic agent does not comprise an antibody or antibody fragment targeting CLDN18.2. In some embodiments, an immunotherapeutic agent does not comprise an antibody or antibody fragment targeting CLDN18.1. In some embodiments, an immunotherapeutic agent does not comprise an antibody or antibody fragment targeting CLDN18.

In some embodiments, a target targeted by an immunotherapeutic agent is a tumor-associated antigen, including fragments thereof such as procession products of a tumor-associated antigen, e.g., antigenic peptides presented on the cell surface in the context of the major histocompatibility complex (MHC).

In some embodiments, an immunotherapy described herein induces lymphocytes such as T cells, e.g., anti-tumor lymphocytes such as T cells. In some embodiments, the lymphocytes are tumor-infiltrating lymphocytes.

### Immunotherapy

As part of its normal function, the immune system detects and destroys abnormal cells and prevents or limits the growth of many cancers. For instance, T lymphocytes are sometimes found in and around tumors. These cells are a sign that the immune system is responding to the tumor. Accumulating evidence shows a correlation between tumor-infiltrating lymphocytes in cancer tissue and favorable prognosis in various malignancies. In particular, the presence of CD8+ cytotoxic T cells seems to correlate with improved prognosis and long-term survival in many solid tumors. T-cell antigen receptors (TCR) on T lymphocytes may engage with antigenic peptides presented on the cell surface in the context of the major histocompatibility complex (MHC). When a T cell encounters a tumor antigen, this results in activation, clonal proliferation/expansion, and a cytolytic response.

The term “immunotherapy” as used herein refers the treatment of a disease or condition by inducing, enhancing, or suppressing an immune response.

Immunotherapy may be used to help the immune system to better act against cancer. Cancer immunotherapy is a form of cancer treatment that uses the patient's immune system to prevent, control, or eliminate cancer.

Types of immunotherapy which may be used to treat cancer include any treatments which induce or enhance an immune response against the cancer. In some embodiments, an immune response, in particular an immune response against cancer described herein refers to the activity such as anti-cancer activity of lymphocytes such as anti-cancer lymphocytes, including tumor-infiltrating lymphocytes (TILs), such as T cells, in particular CD8+ T cells.

Accordingly, an immunotherapy may aim at enhancing the activity such as anti-cancer activity of lymphocytes such as anti-cancer lymphocytes, including tumor-infiltrating lymphocytes, such as T cells, in particular CD8+ T cells. The activity of lymphocytes against cancer may be enhanced by increasing the number of anti-cancer lymphocytes, increasing the reactivity of anti-cancer lymphocytes, and/or increasing tumor infiltration by anti-cancer lymphocytes. Herein, we refer to tumors being infiltrated by CD8+ T as “hot” or “TILs high” and tumours not being infiltrated by CD8+ T cell as “cold” or TILs low”.

Anti-cancer immunotherapies comprise immune checkpoint inhibitors, vaccination, adoptive cell transfer, antibody treatment, and immune system modulators.

### Immune Checkpoint Inhibitors

Immune checkpoint inhibitors are drugs that block immune checkpoints. Immune checkpoints such as PD-1/PD-L1 or CTLA-4 are negative regulators of activated T cells. Using monoclonal antibodies (mAbs) to disrupt the ligand/receptor pairing of these immune checkpoint molecules enables tumor-associated T cells to overcome immunosuppression and effectively perform anti-tumor functions. Immune checkpoint blockade has drastically improved clinical outcomes for patients with cancer.

### Vaccination

Vaccines may boost a patient's immune system to respond to cancer cells. In contrast to immune checkpoint inhibition, where T cells are indiscriminately “rescued” from exhaustion regardless of antigen specificity, cancer vaccines aim to induce a tumor-specific adaptive immune response through delivery of whole tumor cells or tumor-derived antigens (either by administering the antigen directly (peptide vaccines) or encoded by a nucleic acid such as DNA or RNA (DNA vaccines, RNA vaccines)). A notable characteristic is the classification of antigens into shared tumor antigens, which are common between most tumors of a certain histological type, and neoantigens, which are antigens that are subjected to various mutations that render them distinct from normal cells. These neoantigens give rise to neoepitopes, which elicit specific immune cell responses against them.

### Cell Transfer

Cell transfer therapy is a treatment that boosts the natural ability of immune cells such as T cells to fight cancer. In this treatment, tumor-specific immune cells such as T cells which are enriched via ex vivo expansion are given to a patient. The immune cells may be taken from the patient and may optionally be selected or modified to enhance the ability to attack cancer cells.

Such cell transfer therapy may also be called adoptive cell therapy, adoptive immunotherapy, or immune cell therapy.

In addition to tumor infiltrating lymphocytes (TILs), genetically modified T cells can also be used for adoptive cell therapy, including TCR- or CAR-transfected lymphocytes.

### Antibody Treatment

Antibodies, in particular monoclonal antibodies, which bind to cancer cells may be administered to mark cancer cells so that they will be better seen and destroyed by the immune system.

### Immune System Modulators

Immune system modulators may enhance a patient's immune response against cancer. For example, cytokine immunotherapy is an important area of cancer immunotherapy that functions by activating the immune system of patients with cancer. The interleukin-2 (IL-2) family of cytokines comprises IL-2, IL-7, IL-15 and IL-21, and is the most targeted cytokine family in cancer immunotherapy.

### Claudin 18

Claudins are a family of proteins that are the most important components of tight junctions, where they establish the paracellular barrier that controls the flow of molecules in the intercellular space between cells of an epithelium. Claudins are transmembrane proteins spanning the membrane 4 times with the N-terminal and the C-terminal end both located in the cytoplasm. The first extracellular loop or domain consists on average of 53 amino acids, and the second extracellular loop or domain consists of around 24 amino acids. Cell surface proteins of the claudin family are expressed in tumors of various origins, and are suited as target structures in connection with cancer immunotherapy, e.g., antibody-mediated cancer immunotherapy, due to their selective expression (no expression in a toxicity relevant normal tissue) and localization to the plasma membrane.

The term “CLDN” as used herein means claudin. Preferably, a claudin is a human claudin. Claudin 18 (CLDN18) is a member of the claudin family and includes claudin 18 splice variant 1 (claudin 18.1 (CLDN18.1)) and claudin 18 splice variant 2 (claudin 18.2 (CLDN18.2)).

Claudin 18.2 (CLDN18.2) is a 27.8 kDa protein with four membrane-spanning domains and two small extracellular loops (Niimi et al. (2001) Mol Cell Biol. 21(21):7380-90). CLDN18.2 is selectively expressed in normal tissues in differentiated epithelial cells of the gastric mucosa. CLDN18.2 is expressed in various human cancers such as pancreatic carcinoma, esophageal carcinoma, gastric carcinoma, bronchial carcinoma, breast carcinoma, and ENT tumors.

The lung-specific claudin 18.1 (CLDN18.1) is one of the most highly expressed claudin family members in alveolar epithelial cells.

Exemplary sequences of CLDN18.2 (SEQ ID NO: 1) and the splice variant CLDN18.1 (SEQ ID NO: 2) are shown below:

(SEQ ID NO: 1)
MAVTACQGLGFVVSLIGIAGIIAATCMDQWSTQDLYNNPVTAVFN
YQGLWRSCVRESSGFTECRGYFTLLGLPAMLQAVRALMIVGIVLG
AIGLLVSIFALKCIRIGSMEDSAKANMTLTSGIMFIVSGLCAIAG
VSVFANMLVTNFWMSTANMYTGMGGMVQTVQTRYTFGAALFVGWV
AGGLTLIGGVMMCIACRGLAPEETNYKAVSYHASGHSVAYKPGGF
KASTGFGSNTKNKKIYDGGARTEDEVOSYPSKHDYV
(SEQ ID NO: 2)
MSTTTCQVVAFLLSILGLAGCIAATGMDMWSTQDLYDNPVTSVFQ
YEGLWRSCVRQSSGFTECRPYFTILGLPAMLQAVRALMIVGIVLG
AIGLLVSIFALKCIRIGSMEDSAKANMTLTSGIMFIVSGLCAIAG
VSVFANMLVTNFWMSTANMYTGMGGMVQTVQTRYTFGAALFVGWV
AGGLTLIGGVMMCIACRGLAPEETNYKAVSYHASGHSVAYKPGGF
KASTGFGSNTKNKKIYDGGARTEDEVQSYPSKHDYV

In some embodiments, a reference herein to claudin 18 (CLDN18) relates collectively to claudin 18.1 (CLDN18.1) and claudin 18.2 (CLDN18.2). In some embodiments, a reference herein to claudin 18 (CLDN18) relates to claudin 18.1 (CLDN18.1). In some embodiments, a reference herein to claudin 18 (CLDN18) relates to 18.2 (CLDN18.2).

In some embodiments, claudin 18.1 (CLDN18.1) comprises the amino acid sequence of SEQ ID NO: 2 or a variant thereof. In some embodiments, claudin 18.2 (CLDN18.2) comprises the amino acid sequence of SEQ ID NO: 1 or a variant thereof.

### Agent Stabilizing or Increasing Expression of Claudin 18

It is demonstrated herein that increasing CLDN18 expression supports the efficacy of an immune response against cancer. Accordingly, provided herein is a method of enhancing an immune response against cancer in a cancer patient comprising administering an agent stabilizing or increasing expression of claudin 18. The immune response may be a spontaneous (i.e., natural) immune response, an immune response which is induced or enhanced by immunotherapy, or a combination thereof. In some embodiments, provided herein is a combination therapy for effectively treating and/or preventing cancer diseases comprising administering to a patient an agent stabilizing or increasing expression of CLDN18 and an immunotherapy. The agent stabilizing or increasing expression of CLDN18 may be administered prior to, simultaneously with or following administration of the immunotherapy, or a combination thereof.

Certain chemotherapeutic agents, for example gemcitabine, oxaliplatin, and 5-fluorouracil were shown to upregulate existing CLDN18.2 expression levels in cancer cells (WO2013/174510; Tureci O. et al. (2019) OncoImmunology, 8:1).

The term “agent stabilizing or increasing expression of CLDN18” refers to an agent or a combination of agents the provision of which to cells results in increased RNA and/or protein levels of CLDN18, e.g., CLDN18.2, preferably in increased levels of CLDN18, e.g., CLDN18.2, protein on the cell surface, compared to the situation where the cells are not provided with the agent or the combination of agents. Preferably, the cells are cancer cells, in particular cancer cells expressing CLDN18, e.g., CLDN18.2. The term “agent stabilizing or increasing expression of CLDN” refers, in particular, to an agent or a combination of agents the provision of which to cells results in a higher density of CLDN18, e.g., CLDN18.2, on the surface of said cells compared to the situation where the cells are not provided with the agent or the combination of agents. “Stabilizing expression of CLDN18” includes, in particular, the situation where the agent or the combination of agents prevents a decrease or reduces a decrease in expression of CLDN18, e.g., CLDN18.2, e.g. expression of CLDN18, e.g., CLDN18.2 would decrease without provision of the agent or the combination of agents and provision of the agent or the combination of agents prevents said decrease or reduces said decrease of CLDN18, e.g., CLDN18.2, expression. “Increasing expression of CLDN18” includes, in particular, the situation where the agent or the combination of agents increases expression of CLDN18, e.g., CLDN18.2, e.g. expression of CLDN18, e.g., CLDN18.2 would decrease, remain essentially constant or increase without provision of the agent or the combination of agents and provision of the agent or the combination of agents increases CLDN18, e.g., CLDN18.2, expression compared to the situation without provision of the agent or the combination of agents so that the resulting expression is higher compared to the situation where expression of CLDN18, e.g., CLDN18.2, would decrease, remain essentially constant or increase without provision of the agent or the combination of agents.

In some embodiments, the term “agent stabilizing or increasing expression of CLDN18” includes chemotherapeutic agents or combinations of chemotherapeutic agents such as cytostatic agents. In some embodiments, the agent stabilizing or increasing expression of CLDN18 may be a cytotoxic and/or cytostatic agent.

In some embodiments, the term “agent stabilizing or increasing expression of CLDN18” relates to an agent or a combination of agents such a cytostatic compound or a combination of cytostatic compounds the provision of which to cells, in particular cancer cells, results in the cells being arrested in or accumulating in one or more phases of the cell cycle, preferably in one or more phases of the cell cycle other than the G1- and GO-phases, preferably other than the G1-phase, preferably in one or more of the G2- or S-phase of the cell cycle, or a combination thereof, or a combination of the S-phase or the G2-phase with the G1-phase such as the G1/G2-, S/G2-, G2- or S-phase of the cell cycle. The term “cells being arrested in or accumulating in one or more phases of the cell cycle” means that the percentage of cells which are in said one or more phases of the cell cycle increases. Each cell goes through a cycle comprising four phases in order to replicate itself. The first phase called G1 is when the cell prepares to replicate its chromosomes. The second stage is called S, and in this phase DNA synthesis occurs and the DNA is duplicated. The next phase is the G2 phase, when the RNA and protein duplicate. The final stage is the M stage, which is the stage of actual cell division. In this final stage, the duplicated DNA and RNA split and move to separate ends of the cell, and the cell actually divides into two identical, functional cells. Chemotherapeutic agents which are DNA damaging agents usually result in an accumulation of cells in the G1 and/or G2 phase. Chemotherapeutic agents which block cell growth by interfering with DNA synthesis such as antimetabolites usually result in an accumulation of cells in the S-phase. Examples of these drugs are 6-mercaptopurine and 5-fluorouracil.

In some embodiments, the agent stabilizing or increasing expression of CLDN18 may comprise an agent selected from the group consisting of anthracyclines, nucleoside analogs, platinum compounds, camptothecin analogs and taxanes, prodrugs thereof, salts thereof, and combinations thereof. The anthracycline may be selected from the group consisting of epirubicin, doxorubicin, daunorubicin, idarubicin, valrubicin, prodrugs thereof and salts thereof. The anthracycline may be selected from the group consisting of epirubicin, prodrugs thereof and salts thereof. Preferably, the anthracycline is epirubicin. The nucleoside analog may be selected from the group consisting of gemcitabine, 5-fluorouracil, prodrugs thereof and salts thereof. The platinum compound may selected from the group consisting of oxaliplatin, cisplatin, prodrugs thereof and salts thereof. The camptothecin analog may be selected from the group consisting of irinotecan, topotecan, prodrugs thereof and salts thereof. The taxane may be selected from the group consisting of paclitaxel, docetaxel, prodrugs thereof and salts thereof.

In some embodiments, a reference to an agent stabilizing or increasing expression of CLDN18, such as a reference to an anthracycline, a nucleoside analog, a platinum compound, a camptothecin analog or a taxane, for example, a reference to gemcitabine, 5-fluorouracil, oxaliplatin, irinotecan or paclitaxel is to include any prodrug such as ester, salt or derivative such as conjugate of said agent. Examples are conjugates of said agent with a carrier substance, e.g. protein-bound paclitaxel such as albumin-bound paclitaxel. Preferably, salts of said agent are pharmaceutically acceptable.

In some embodiments, an “agent stabilizing or increasing expression of CLDN18” comprises an “agent inducing immunogenic cell death”.

In specific circumstances, cancer cells can enter a lethal stress pathway linked to the emission of a spatiotemporally defined combination of signals that is decoded by the immune system to activate tumor-specific immune responses (Zitvogel L. et al. (2010) Cell 140: 798-804). In such scenario cancer cells are triggered to emit signals that are sensed by innate immune effectors such as dendritic cells to trigger a cognate immune response that involves CD8+ T cells and IFN-γ signalling so that tumor cell death may elicit a productive anticancer immune response. These signals include the pre-apoptotic exposure of the endoplasmic reticulum (ER) chaperon calreticulin (CRT) at the cell surface, the pre-apoptotic secretion of ATP, and the post-apoptotic release of the nuclear protein HMGB1. Together, these processes constitute the molecular determinants of immunogenic cell death (ICD). Anthracyclines, oxaliplatin, and γ irradiation are able to induce all signals that define ICD, while cisplatin, for example, which is deficient in inducing CRT translocation from the ER to the surface of dying cells -a process requiring ER stress -requires complementation by thapsigargin, an ER stress inducer.

As used herein, the term “agent inducing immunogenic cell death” refers to an agent or a combination of agents which when provided to cells, in particular cancer cells, is capable of inducing the cells to enter a lethal stress pathway which finally results in tumor-specific immune responses. In particular, an agent inducing immunogenic cell death when provided to cells induces the cells to emit a spatiotemporally defined combination of signals, including, in particular, the pre-apoptotic exposure of the endoplasmic reticulum (ER) chaperon calreticulin (CRT) at the cell surface, the pre-apoptotic secretion of ATP, and the post-apoptotic release of the nuclear protein HMGB1.

As used herein, the term “agent inducing immunogenic cell death” includes anthracyclines such as epirubicin and oxaliplatin.

The term “nucleoside analog” refers to a structural analog of a nucleoside, a category that includes both purine analogs and pyrimidine analogs.

The term “gemcitabine” is a compound which is a nucleoside analog of the following formula:

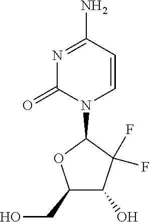 [SMILES: Nc1ccn([C@@H]2O[C@H](CO)[C@@H](O)C2(F)F)c(=O)n1]

In particular, the term refers to the compound 4-amino-1-(2-deoxy-2,2-difluoro-β-D-erythro-pentofuranosyl)pyrimidin-2(1H)-one or 4-amino-1-[(2R,4R,5R)-3,3-difluoro-4-hydroxy-5-(hydroxymethyl)oxolan-2-yl]-1,2-dihydropyrimidin-2-one.

The term “nucleoside analog” includes fluoropyrimidine derivatives such as fluorouracil and prodrugs thereof. The term “fluorouracil” or “5-fluorouracil” (5-FU or f5U) (sold under the brand names Adrucil, Carac, Efudix, Efudex and Fluoroplex) is a compound which is a pyrimidine analog of the following formula:

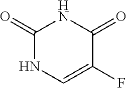

In particular, the term refers to the compound 5-fluoro-1H-pyrimidine-2,4-dione.

The term “capecitabine” (Xeloda, Roche) refers to a chemotherapeutic agent that is a prodrug that is converted into 5-FU in the tissues. Capecitabine which may be orally administered has the following formula:

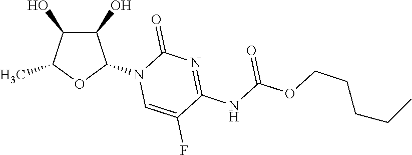 [SMILES: CCCCCOC(=O)Nc1nc(=O)n([C@@H]2O[C@H](C)[C@@H](O)[C@H]2O)cc1F]

In particular, the term refers to the compound pentyl [1-(3,4-dihydroxy-5-methyltetrahydrofuran-2-yl)-5-fluoro-2-oxo-1H-pyrimidin-4-yl]carbamate.

The term “platinum compound” refers to compounds containing platinum in their structure such as platinum complexes and includes compounds such as cisplatin, carboplatin and oxaliplatin.

The term “cisplatin” or “cisplatinum” refers to the compound cis-diamminedichloroplatinum(II) (CDDP) of the following formula:

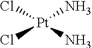

The term “carboplatin” refers to the compound cis-diammine(1,1-cyclobutanedicarboxylato)platinum(II) of the following formula:

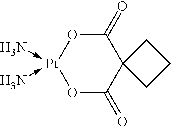

The term “oxaliplatin” refers to a compound which is a platinum compound that is complexed to a diaminocyclohexane carrier ligand of the following formula:

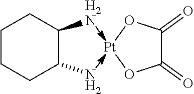

In particular, the term “oxaliplatin” refers to the compound [(1R,2R)-cyclohexane-1,2-diamine](ethanedioato-O,O′)platinum(II). Oxaliplatin for injection is also marketed under the trade name Eloxatine.

Taxanes are a class of diterpene compounds that were first derived from natural sources such as plants of the genus Taxus, but some have been synthesized artificially. The principal mechanism of action of the taxane class of drugs is the disruption of microtubule function, thereby inhibiting the process of cell division. Taxanes include docetaxel (Taxotere) and paclitaxel (Taxol).

The term “docetaxel” refers to a compound having the following formula:

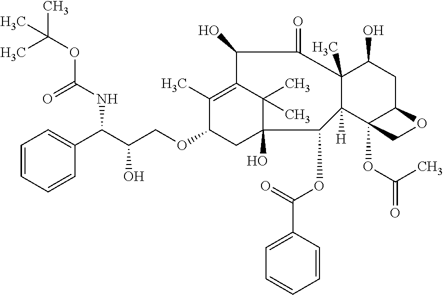 [SMILES: CC(=O)O[C@@]12CO[C@@H]1C[C@H](O)[C@@]1(C)C(=O)C[C@H](O)C3=C(C)[C@@H](OC[C@H](O)[C@@H](NC(=O)OC(C)(C)C)c4ccccc4)C[C@@](O)([C@@H](OC(=O)c4ccccc4)[C@H]21)C3(C)C]

In particular, the term “docetaxel” refers to the compound 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)-amino]-2-hydroxy-3-phenylpropanoate}.

The term “paclitaxel” refers to a compound having the following formula:

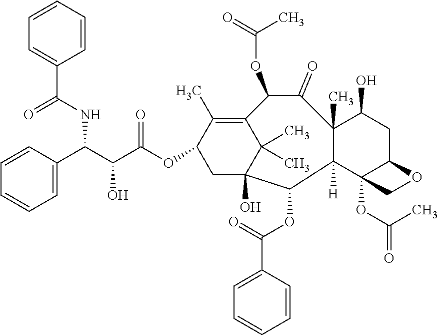 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

In particular, the term “paclitaxel” refers to the compound (2α,4α,5β,7β,10β31α)-4,10-bis-(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate.

The term “camptothecin analog” refers to derivatives of the compound camptothecin (CPT; (S)-4-ethyl-4-hydroxy-1H-pyrano[3′,4′:6,7]indolizino[1,2-b]quinoline-3,14-(4H,12H)-dione).

Preferably, the term “camptothecin analog” refers to compounds comprising the following structure:

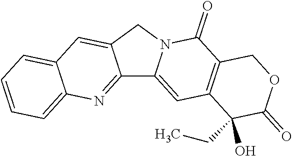 [SMILES: CC[C@@]1(O)C(=O)OCc2c1cc1n(c2=O)Cc2cc3ccccc3nc2-1]

Preferred camptothecin analogs are inhibitors of DNA enzyme topoisomerase I (topo 1).

Preferred camptothecin analogs are irinotecan and topotecan.

Irinotecan is a drug preventing DNA from unwinding by inhibition of topoisomerase I. In chemical terms, it is a semisynthetic analogue of the natural alkaloid camptothecin having the following formula:

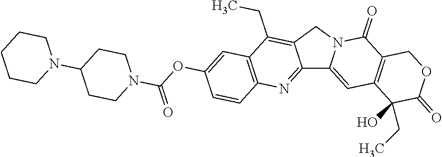 [SMILES: CCc1c2c(nc3ccc(OC(=O)N4CCC(N5CCCCC5)CC4)cc13)-c1cc3c(c(=O)n1C2)COC(=O)[C@]3(O)CC]

In particular, the term “irinotecan” refers to the compound (S)-4,11-diethyl-3,4,12,14-tetrahydro-4-hydroxy-3,14-dioxo1H-pyrano[3′,4′:6,7]-indolizino[1,2-b]quinolin-9-yl-[1,4′-bipiperidine]-1′-carboxylate.

Topotecan is a topoisomerase inhibitor of the formula:

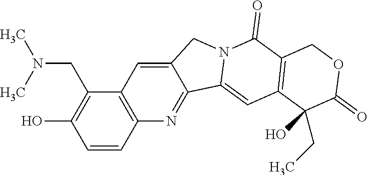 [SMILES: CC[C@@]1(O)C(=O)OCc2c1cc1n(c2=O)Cc2cc3c(CN(C)C)c(O)ccc3nc2-1]

In particular, the term “topotecan” refers to the compound (S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride.

Anthracyclines are a class of drugs commonly used in cancer chemotherapy that are also antibiotics. Structurally, all anthracyclines share a common four-ringed 7,8,9,10-tetrahydrotetracene-5,12-quinone structure and usually require glycosylation at specific sites.

Anthracyclines preferably bring about one or more of the following mechanisms of action: 1.

Inhibiting DNA and RNA synthesis by intercalating between base pairs of the DNA/RNA strand, thus preventing the replication of rapidly-growing cancer cells. 2. Inhibiting topoisomerase 11 enzyme, preventing the relaxing of supercoiled DNA and thus blocking DNA transcription and replication. 3. Creating iron-mediated free oxygen radicals that damage the DNA and cell membranes.

The term “anthracycline” preferably relates to an agent, preferably an anticancer agent for inducing apoptosis, preferably by inhibiting the rebinding of DNA in topoisomerase II.

Preferably, the term “anthracycline” generally refers to a class of compounds having the following ring structure

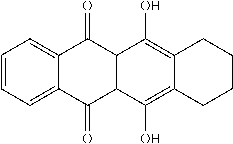 [SMILES: O=C1c2ccccc2C(=O)C2C(O)=C3CCCCC3=C(O)C12]

including analogs and derivatives, pharmaceutical salts, hydrates, esters, conjugates and prodrugs thereof.

Examples of anthracyclines and anthracycline analogs include, but are not limited to, daunorubicin (daunomycin), doxorubicin (adriamycin), epirubicin, idarubicin, rhodomycin, pyrarubicin, valrubicin, N-trifluoro-acetyl doxorubicin-14-valerate, aclacinomycin, morpholinodoxorubicin (morpholino-DOX), cyanomorpholino-doxorubicin (cyano-morpholino-DOX), 2-pyrrolino-doxorubicin (2-PDOX), 5-iminodaunomycin, mitoxantrone and aclacinomycin A (aclarubicin). Mitoxantrone is a member of the anthracendione class of compounds, which are anthracycline analogs that lack the sugar moiety of the anthracyclines but retain the planar polycylic aromatic ring structure that permits intercalation into DNA.

Particularly preferred as anthracyline is a compound of the following formula:

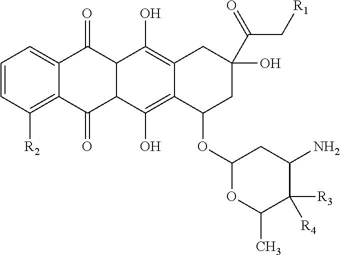

wherein

R₁ is selected from the group consisting of H and OH, R₂ is selected from the group consisting of H and OMe, R₃ is selected from the group consisting of H and OH, and R₄ is selected from the group consisting of H and OH.

In one embodiment, R₁ is H, R₂ is OMe, R₃ is H, and R₄ is OH. In another embodiment, R₁ is OH, R₂ is OMe, R₃ is H, and R₄ is OH. In another embodiment, R₁ is OH, R₂ is OMe, R₃ is OH, and R₄ is H. In another embodiment, R₁ is H, R₂ is H, R₃ is H, and R₄ is OH.

Specifically contemplated as anthracycline is epirubicin. Epirubicin is an anthracycline drug which has the following formula:

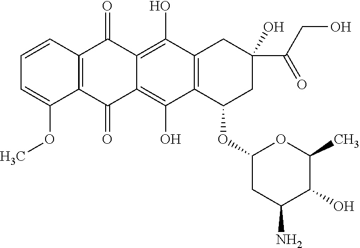 [SMILES: COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1]

and is marketed under the trade name Ellence in the US and Pharmorubicin or Epirubicin Ebewe elsewhere. In particular, the term “epirubicin” refers to the compound (8R,10S)-10-[(2S,4S,5R,6S)-4-amino-5-hydroxy-6-methyl-oxan-2-yl]oxy-6,11-dihydroxy-8-(2-hydroxyacetyl)-1-methoxy-8-methyl-9,10-dihydro-7H-tetracen-5,12-dion. Epirubicin is favoured over doxorubicin, the most popular anthracycline, in some chemotherapy regimens as it appears to cause fewer side-effects.

### Pharmaceutical Compositions

The agents, e.g., immunotherapeutic agents and agent stabilizing or increasing expression of CLDN18, described herein may be administered in pharmaceutical compositions and may be administered in the form of any suitable pharmaceutical composition.

In some embodiments, the agents described herein may be administered in a pharmaceutical composition which may comprise a pharmaceutically acceptable carrier and may optionally comprise one or more stabilizers etc. In some embodiments, the pharmaceutical composition is for therapeutic or prophylactic treatments, e.g., for use in treating or preventing a disease. The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions according to the present disclosure are generally applied in a “pharmaceutically effective amount” and in “a pharmaceutically acceptable preparation”. The term “pharmaceutically acceptable” refers to the non-toxicity of a material which does not interact with the action of the active component of the pharmaceutical composition. The term “pharmaceutically effective amount” or “therapeutically effective amount” refers to the amount which achieves a desired reaction or a desired effect alone or together with further doses and/or agents. In the case of the treatment of a particular disease, the desired reaction preferably relates to inhibition of the course of the disease. This comprises slowing down the progress of the disease and, in particular, interrupting or reversing the progress of the disease.

The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient.

Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In some embodiments, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the pharmaceutical composition is formulated for systemic administration, e.g., for intravenous administration.

The present invention is further illustrated by the following examples which are not be construed as limiting the scope of the invention.

### EXAMPLES

### Methods

### Animals

C57Bl/6 mice were purchased from Charles River Laboratories Inc; TCR-transgenic mice C57BL/6-Tg(TcraTcrb)1100Mjb/J (H-2^{b}, referred as OT-1) and RAG2 KO -B6.Cg-Rag2tm1.1Cgn/J were purchased from Jackson Laboratory (Bar Harbor, Maine, USA). NOG mice (NOD Cg-Prkdcscid Il2rgtmlSug/JicTac) were purchased from Taconic Biosciences (NY, USA). Animals were maintained in individually ventilated cages and in pathogen-free conditions at the animal facility of the University of Verona under standardized conditions with a 12-h photoperiod and were provided with food and water ad libitum. All genetically transgenic mice and their respective controls were gender and age-matched (typically 8-10 weeks).

### Cell Lines and Culture Conditions

Mouse FC1199 (H-2b) and FC1242 (H-2b) KPC-derived cell lines were kindly donated by Dr. D. Tuveson (Cold Spring Harbor Laboratory, NY, USA). FC1199, FC1242, as well as MBL-2 and HEK293 (ATCC-Manassas, VA), were cultured in high-glucose DMEM supplemented with 10% heat-inactivated fetal bovine serum, 2 mM L-glutamine, 10 mM HEPES, 10 mM sodium pyruvate, 150 U/ml streptomycin and 200 U/ml penicillin. hTERT-specific T cells or OT-I-derived splenocytes were respectively cultured in RPMI 1640 or high-glucose DMEM supplemented with 10% heat-inactivated fetal bovine serum, 2 mM L-glutamine, 10 mM HEPES, 10 mM sodium pyruvate, 20 μM β-mercaptoethanol, 100 U/ml streptomycin, 100 U/ml penicillin and 0.1 mM MEM Non-Essential Amino Acid Solution.

Human pancreatic cancer cell lines DANG and DANG-CLDN18 (overexpressing the predominantly expressed in pancreatic cancers isoform CLDN18.2) were a kind gift from the Dr.ssa Ö. Tiireci and Dr. U. Sahin. Cell lines were cultured with in RPMI 1640 supplemented with 10% heat-inactivated FBS, 2 mM L-glutamine, 10 mM HEPES, 150 U/ml streptomycin and 200 U/ml penicillin. The cell culture medium for DANG-CLDN18 was additionally supplemented with 1 μg/ml blasticidin. All cell lines were thawed from primary stocks maintained under liquid nitrogen and cultured for a maximum of three weeks during which time all experiments were performed. All cell lines were culture in 5% C02-humidified incubator at 37° C. and routinely validated to be free from mycoplasma by PCR.

### CRISPR and Single-Cell-Derived Clone Preparation

For the CLDN18 knockout in FC1199-OVA cells, specific crRNA was designed, targeting the sequence in the second exon of the mouse CLDN18 gene, and purchased from Integrated DNA Technologies (IDT, Iowa, USA). Cas9 protein and tracrRNA were purchased from the same company. gRNA was prepared by mixing in equimolar ration crRNA and tracrRNA and incubation for 5 minutes at 95° C. Equimolar ratios of gRNA and Cas9 were mixed and incubated with Viromer CRISPR reagent for ribonucleoproteins (RNP) formation. The RNP complexes were added to FC1199-OVA cells, next day the cells were seeded in 96-well plate at 1 cell per well dilution. Single-cell-derived clones were detected and cultured for 2 weeks for amplification. CLDN18 loss was determined by FACS analysis.

### Activated OVA-Specific T Cell Preparation

Splenocytes from OT-I TCR transgenic mice were isolated by mechanical spleen smashing through a 70 m nylon mesh filter to obtain a single-cell suspension. Briefly, 10×10⁶ cells per well were cultured in a 24-well plate for 3 days in the presence of 1 μg/ml OVA257-264 peptide (SIINFEKL), supplemented every day with fresh medium and 20 U/ml of human recombinant IL-2. Purity of CD8⁺ Vβ5⁺ CTLs was determined by FACs analysis. For immunological synapse formation and live imaging experiments, OVA-specific CTLs were washed with culture medium and maintained in resting conditions for the last 24 h before the experiment supplemented with 20 U/ml of human recombinant IL-2.

Generation of hTERT-Specific T Cells

hTERT-specific T cells were generated as previously described (Sandri et al., (2016) Cancer Res 76, 2540-2551). AIM-V medium (Gibco) supplemented with 5% human serum (Gibco, Thermo Fisher Scientific, Waltham, MA, USA) was used for clone preparation and expansion. Briefly, PBMCs were activated on plates pre-coated with anti-CD3 activating antibody (Thermo Fisher Scientific) and infected with hTERT₈₆₅₋₈₇₃/PG13 cell-derived viral supernatant. Transduced cells were sorted with CD34-selection beads (Miltenyi Biotec, Germany) and expanded in presence of 300 IU/ml human recombinant IL-2 (Miltenyi Biotec, Germany) and 100 μg/ml of human recombinant IL-15 (Miltenyi Biotec, Germany).

### Lentivirus-Mediated Transduction in Tumor Cells

The lentiviral construct pELNS-OVA-GFP was kindly provided by Dr. G. Coukos group. CLDN18-His was subcloned in pELNS to generate pELNS-GFP-CLDN18-His. For virus productions HEK293 cells were used as packaging cells. The HIV-l lentiviral packaging was performed in HEK293 cells, where lentiviral vectors along with pDEL, pREV, and VSV-G were delivered by incubation with 60 mM CaCl₂. Virus-containing supernatants were harvested 48 h after transfection, filtered with 0.22 m-pored filters and concentrated by ultracentrifugation at 50.000 g for 2.20 h. Target cell lines were infected with MOI (multiplicity of infection) 2 in presence of 8 μg/ml of polybrene (Millipore, Billerica, MA, USA) and subsequently cultured for 24 h at 37° C. After 2 days of infection the cell lines were sorted twice for the enrichment of GFP-positive cells using FACS Aria II Flow Cytometer Cell Sorter (BD Biosciences).

siRNA Silencing 1×10⁵ cancer cells were let seed in 24-well plate for 5h, before performing the transfection (TransIT-X2®—Myrus). After 45 hours, medium was replaced in each well with 450 μl of complete growth medium. Transfection mix comprises 50 μl Opti-MEM, 14 pmole of ALCAM siRNA (siALCAM) or irrelevant siRNA (siSCR) and 1 μl of Transit. After incubation 50 μl of transfection mix were added per well, drop-wise and next day 500 μl of complete medium was added on top. ALCAM silencing was evaluated by flow cytometry.

### In Vivo Tumor Models

5×10⁵, 5×10⁴, 2×10⁴ tumor cells were used for subcutaneous, orthotopic and intravenous syngeneic tumor studies, respectively. Subcutaneous tumor growth was monitored every 2 days using a digital caliper and metastasis were evaluated by lung weight. The greatest longitudinal diameter (length) and the greatest transverse diameter (width) were determined and tumor volume was calculated by the modified ellipsoidal formula: tumor volume=½ (length×width²). 1×10⁶ tumor cells were subcutaneously injected in xenogeneic models (DANG, DANG-CLDN18 tumor cell lines).

### In Vivo Treatments

Adoptive cell therapy. OVA-specific CTLs were adoptively transferred by intravenous injection in tumor bearing mice at dose comprised between 2×10⁶ and 1×10⁵ cells according to the experiment. 5×10⁶ hTERT-specific T lymphocytes were adoptively transferred by intravenous injection in mice bearing human tumor cell lines.

In vivo transduction. 5×10⁶ and 3×10⁷ pELNS-GFP-CLDN18-His lentivirus particles encoding a CLDN18-GFP fusion construct were intratumorally injected in FC1242-OVA tumor bearing mice.

In vivo ALCAM blockade. CLDN18^{+/+} and CLDN18^{-/-} tumor bearing mice were administrated 3 hours prior to ACT by 200 μg intraperitoneal plus 50 μg intratumor delivery of monoclonal antibody anti-ALCAM or isotype control.

### Intratumoral pH Measurement

pH meter with a needle probe was used directly on tumors 14 days after injection.

### Bioluminescence Imaging

In the spontaneous and immunogenic model of lung adenocarcinoma in CLDN18 proficient and CLDN18^{+/+} mice, tumor growth was monitored by bioluminescence imaging (BLI; photons/second/cm²/sr) using the IVIS Spectrum Imaging System (Perkin Elmer, Waltham, MA, USA). Lung bioluminescence images were acquired ex-vivo and tumor burden was evaluated 18 weeks after viral instillation. The subsequent parameters were used: exposure time=5 minutes, field of view=19×19 cm, binning B=8 and f/stop=1. Images were quantified tracing the region of interest (ROI) on the entire animal body. Living Image Software 4.4 (Perkin Elmer, Waltham, MA, USA) was used to acquire and quantify the bioluminescence.

Preparation of Cell Suspensions from Mouse Organs

Donor tumor bearing mice were sacrificed and the spleens and tumors were collected and processed according to already published protocols (Facciabene et al., (2017) Oncoimmunology 6, e1326442). Briefly, spleens were mechanically homogenized, red blood cells were lysed and cell suspension was filtered on cell strainer (Coming Inc, New York, USA) to remove aggregates. Tumors were finely cut and enzymatically digested with a solution containing 75 mg/ml collagenase type I, 75 mg/ml collagenase type II, 50 mg/ml collagenase type IV, 10 mg/ml DNAse I and 10 mg/ml elastase in DMEM medium for 1 hour at 37° C. pipetting every 15 minutes. Tumor cell suspension was separated from aggregates by 70 μm cell strainer filtration, washed with complete DMEM media and, if necessary, deprived of red blood cells. Finally, cells were utilized for flow cytometry staining.

### Tumor Cell Line Proliferation Assay

FC1199, FC1199-OVA, FC1242 and FC1242-OVA cells were seeded in 6-well plates at concentration of 2.5×10⁵ cells/well. Every 24 h cell number was assessed by detaching cells, counterstaining the dead cells with trypan blue solution (Lonza).

ELISPOTDonor tumor bearing mice were euthanized and the spleens were harvested after four days from ACT. Single-cell suspensions of splenocytes were used for anti-IFN-γ ELISPOT assay, as previously described (Facciabene et al., (2017) Oncoimmunology 6, el326442). Briefly, 10⁶ splenocytes were seeded on IFNγ-precoated (BD PharMingen) 96-well MAIP plates (Millipore) and incubated at 37° C. for 20 h with 1 g/mL OVA peptide or control H-2Kb-restricted peptide mTERT198-205. Concanavalin A (Sigma) or anti-CD3 (eBioscience) was used at 5 μg/ml as a positive control. Plates were then incubated for 12 h at 4° C. with rat anti-mouse biotin-conjugated IFNγ (BD PharMingen), followed by 3 h at 25° C. with streptavidin-AKP (BD PharMingen) and finally with NBT/BCIP (Pierce) for color development. An ELISPOT reader (AID, Germany) was used to count the spots.

### Chromium Release Assay

Target cells were radioactively labeled by 1 h incubation with hexavalent chromium-51 (Beckman Coulter). MBL-2 cells were pulsed with hTERT or OVA peptide as positive and negative controls. OVA-specific or mTERT cells were prepared as indicated above. T cells and target cells were mixed at different ratios and incubated for 5 h at 37° C. Subsequently, the supernatants were collected and placed for registration in Luma Plates (Perkin Elmer).

Gamma radiation emission was measured as “counts per minute” (cpm) for each well with TopCount NXT™ Microplate Scintillation and Luminescence Counter (Perkin Elmer). To measure maximum ept level (cpm^{max}) cells were lysed by addition of 1% Triton X-100 in water. Culture medium alone was used as negative control to quantify spontaneous radiation emission (cpm^{spont}). Specific lysis in experimental wells was calculated with the formula:

$$\% lysis = \frac{{cpm}^{exp} - {cpm}^{spont}}{{cpm}^{max} - {cpm}^{spont}} \times 100 \%$$

Adhesion Assay with Spheroids

Spheroid formation was performed in 6-well plates. Agar Noble 4% was pre-warmed and mixed with normal DANG culture RPMI medium in 1:1 ratio and 1 ml was layered on one the plate well and solidified. DANG and DANG-CLDN18 cells were plated on top of the agar in normal RPMI culture medium for 3 days or longer until average spheroid diameter reached 210 μm. Single spheroids were size-selected and picked under a microscope and transferred on agar-layered 24 well-plate, 10 spheroids per well. hTERT-specific T cells were pre-stained with CellTrace (Thenno Fisher Scientific MA -USA) and 1×10⁶ were seeded on top of the agar and cultured for 4 h. Afterward, the spheroids were extensively washed in PBS to remove the unattached T cells and were plated in complete white RPMI in chamber slide with matrigel. Spheroids were counterstained with DAPI and chamber slides were imaged with Leica TCS SP5 AOBS confocal-multiphoton system. To enumerate T lymphocytes attached to spheroids, ten organoids were pooled in the same well and GFP quantified by IVIS spectrum Imaging System (Perkin Elmer, Waltham, MA, USA).

### T Cell Migration Assay

DANG and DANG-CLDN18 cells were plated in T75 flask and condition media was collected 72 hours later when cell confluence achieved 80%. CFSE labeled hTERT-specific T cells were seeded on 5 μm-pored transwells (Coming, NY -USA) placed in wells with cancer cell supernatants. CD3-CD28 stimulated T cell conditioned medium was used as a positive control, whereas complete RPMI was used as a negative control. T cells were allowed to migrate for 5 h then cells were detached with PBS containing 0.2% EDTA and migrated cells were counted with BD Trucount™ Absolute Counting Tubes (BD, NJ, USA).

Competition Immunological Synapse Formation CLDN18^{+/+} clones were stained with Cell trace. Different FC1199-OVA CLDN18^{+/+} and CLDN18^{-/-} cell clones were mixed each other in 1:1 ratio. OVA-specific CTLs and target cells were mixed 1:1 with tumor cells and immediately plated on polylysine-coated coverslips in a 24-well plate. Cells were incubated for 1 h at 37° C. and then fixed in 4% PFA for 15 min at RT, before immunofluorescence staining. Fixed cells were permeabilized in Triton-X-100 0.1% in PBS for 30 min at RT, blocked for 2 h at RT in 20% normal goat serum in permeabilization solution, then incubated overnight at 4° C. with BV421-conjugated antibody anti-mCD3 diluted in PBS. Samples were washed with Tween-20 0.05% in PBS before confocal microscopy acquisition.

Immunological synapse was identified as the site of CD3 accumulation on T cell at the contact site with cancer cell. The number of immunological synapses (Nsyn) was counted in each sample for CLDN18^{+/+} cells and CLDN18^{-/-} cells, and normalized to the total number of CLDN18⁺ cells and CLDN18⁻ cells respectively (Ntot). Nine fields were analyzed for each sample. That is described by formulae:

$$\% {CLDN 18}^{+ / +} Cancer cells forming synapses = \frac{Nsyn {CLDN 18}^{+ / +}}{Ntot {CLDN 18}^{+ / +}} ;$$

$$\% {CLDN 18}^{- / -} Cancer cells forming synapses = \frac{Nsyn {CLDN 18}^{- / -}}{Ntot {CLDN 18}^{- / -}} .$$

### Molecular Analysis

Gene expression analysis on microarray and bulk RNA-seq data For microarray analysis, we prepared n=3 biological replicates of FC 1199-OVA and n=3 FC1242-OVA lines. Total RNA was extracted using RNeasy Mini Kit (Qiagen, Hilden, Germany), and contaminant DNA was removed by RNase-Free DNase Set (Qiagen, Hilden, Germany). RNA quality and purity were assessed on the Agilent Bioanalyzer 2100 (Agilent Technologies, Milano, Italy); RNA concentration was determined using the NanoDrop ND-1000 Spectrophotometer (NanoDrop Technologies). Labeling and hybridization were performed according to Affymetrix One Cycle Target Labeling protocol on HG-U133 Plus 2.0 arrays (Affymetrix, Thermo Fisher Scientific, Waltham, MA, USA). The analysis of microarray and bulk RNA-seq data was performed with the R/Bioconductor platform. The raw probe level signals of the microarray were processed for background correction, normalization and summarization using the robust multichip average algorithm (RMA) (Bolstad et al., (2003) Bioinformatics 19, 185-193; Irizarry et al., (2003a) Nucleic Acids Res 31, e15; Irizarry et al., (2003b) Biostatistics 4, 249-264). Differential gene expression analysis between FC1 199-OVA and FC1242-OVA lines was performed using limma package (Ritchie et al., (2015) Nucleic Acids Res 43, e47). Only the genes with an adjusted p-value<0.05 were considered statistically significant. Probes with ambiguous differential behavior were removed from the downstream analyses. Pathway analysis was performed using DAVID web application (Dennis et al., (2003) Genome Biol 4, P3) on the Gene Ontology (GO FAT) categories using the following procedure. (1) A first enrichment analysis was performed using both up- and down-regulated genes in the comparison between FC1199-OVA vs FC1242-OVA to retrieve the regulated biological processes (BP) and cellular components (CC). A separate analysis was performed with only up-(2) or down-regulated (3) genes. (4) For each list of enriched processes, only the categories obtained also in (1) were retained. (5) The list of categories obtained in (2) and (3) were further filtered removing those categories present in both lists and having a smaller number of enriched genes compared to the other. In all the previous steps only the GO categories with an FDR<0.05 were considered statistically significant. Scores on gene sets both for the microarray and bulk RNA-seq data were calculated using gene set variation analysis (GSVA) (Hanzelmann et al., (2013) BMC Bioinformatics 14, 7). Survival analyses were performed using Kaplan-Meier curves. Cut points for stratifying patients were estimated using the maximally selected rank statistics implemented in the R package ‘maxstat’ (https://CRAN.R-project.org/package=maxstat). The R packages ‘survminer’ (https://CRAN.R-project.org/package=survminer) and ‘RTCGA’ (https://rtcga.github.io/RTCGA) were also used for the survival analyses. The comparison of survival curves was performed using logrank p-values considering statistically significant only comparisons with a p-value<0.05. The expression of CLDN18 was analyzed on the PAAD, LUAD and LUSC TCGA datasets (Cancer Genome Atlas Research, (2017) Cancer Cell 32, 185-203 el13) annotated with PDAC (Bailey et al., (2016) Nature 531, 47-52; Collisson et al., (2011) Nat Med 17, 500-503; Moffitt et al., (2015) Nat Genet 47, 1168-1178) and NSCLC molecular subtypes (Chen et al., (2017) Oncogene 36, 1384-1393). The TCGA datasets were obtained from the R package ‘curatedTCGAData’ (Ramos et al., (2020) JCO Clin Cancer Inform 4, 958-971) selecting the normalized RSEM gene expression assays. Associations between CLDN18 expression and ‘EMT’, ‘Signaling by MET’, ‘Hippo signaling pathway’, ‘HIF-1 signaling pathway’, ‘Antigen processing’, ‘Adherens junction’ and ‘Cell-cell communication’ in the Bailey et al. dataset were estimated using linear regression models. The Spearman correlation between CLDN18 and CD3E expression was performed using the R package ‘ggpubr’ (https://CRAN.R-project.org/package=ggpubr). GSVA scores on phenotypic and functional gene signatures (Rooney et al., (2015) Cell 160, 48-61) and T cells low and T cell high tumor samples (Li et al., (2018) Immunity 49, 178-193 e177) were averaged among FC1199-OVA and FC1242-OVA replicates and showed as heatmaps. The mapping between mouse and human gene symbols was performed using the R package biomaRt (Durinck et al., (2005) Bioinformatics 21, 3439-3440).

### Real Time-PCR

Total RNA from FC1199-OVA and FC1242-OVA tumors was isolated from immunocompromised mice that either received or not ACT. Samples were preserved in RNAlater solution (Thermo Fisher Scientific MA -USA) and kept on ice. Before the RNA extraction procedure, the RNAlater solution was removed and tripleXtractor (Grisp) was added. The tumor pieces were homogenized (GentleMacs-Miltenyi Biotec) and incubated for 5 minutes at room temperature. Total RNA was extracted by chloroform and its amount and purity was analyzed by the ND-1000 Spectrophotometer (NanoDrop Technologies). cDNA was generated from 1 ug RNA using RevertAid RT Reverse Transcription Kit (Thermo Fisher Scientific MA -USA) according to the manufacturer's instruction. Semiquantitative real-time PCR was run using SYBR® Green PCR Master Mix (Thermo Fisher Scientific MA -USA).

All samples were normalized on Gapdh housekeeping gene. Post-qRT-PCR analysis to quantify relative gene expression was performed by the comparative Ct method (2-A Ct).

### Flow Cytometry

For the extracellular staining 1×10⁶ cells were blocked with Fc blocking anti-mouse CD16/32 prior to staining (Biolegend) or anti-human (Miltenyi Biotec) for 10 minutes at RT. The following anti-mouse mAbs were then used for 30 min at 4° C.: CD11 b (M1/70), CD45 (30F-11), CD3 (17A2), CD8a (53-6.7), TCR Vβ5.1,5.2 (MR9-4), MHC-I (SF1-1.1), ICAM-1 (YN1/1.7.4), ALCAM (eBioALC48), E-cadherin (DECMA-1), CD62P (Psel.K02.3), VCAM-1 (429 MVCAM.A), PSGL-1 (4RA10), CLDN18 (Imab 362 kindly provided by Dr.ssa 0. Tdreci and Dr. U. Sahin), LIVE/DEAD dyes (ThennoFisher Scientific). For human cells was used the following anti-human hCD3 (SK7; Cat #557832). All the antibodies were purchased from the following companies: BD Bioscience (San Jose, CA, USA), eBiosciences (ThermoFisher Scientific, Waltham, MA, USA), Bio-rad Laboratories (Hercules, CA, USA) and Biolegend (San Diego, CA, USA). For the intracellular staining 1×10⁶ cells were fixed with eBioscience™ Foxp3/Transcription Factor Fixation/Permeabilization Concentrate and Diluent (ThermoFisher Scientific) and permeabilized with Permeabilization Buffer ((ThennoFisher Scientific) according to the product datasheet. The following Ab was then used for 30 min at RT in permeabilization buffer after FcR blocking reagents for 15 min at RT: CLDN18 polyclonal antibody (ThermoFisher Scientific). Samples were acquired with FACS Canto II (BD, Franklin Lakes, NJ, USA) and analyzed by FlowJo software (Tree Star, Inc., Ashland, OR, USA).

### Western-Blot and Quantification

Whole cell lysates of FC1199, FC1199-OVA, FC1242 and FC1242-OVA cells were generated in 200 μl TRITON buffer containing 0.5% Triton X-100, 50 mM Hepes, 150 mM NaCl, 5 mM EDTA, 1 mM NaOV4, 2 mM PMSF, and protease inhibitors. Samples were incubated on ice for 15 minutes then subjected to BCA protein quantification (ThermoFisher Scientific).

Samples were prepared in Laemmli Buffer supplemented with 10% β-mercaptoethanol and denatured at 97° C. for 7 min. Insoluble materials were removed by centrifugation. Samples were subjected to SDS polyacrylamide 10% Tris-Glycine or Bis-Tris gel electrophoresis and blotted onto PVDF-membrane (Immobilon P membranes, Millipore, Billerica, MA, USA).

Tris-buffered saline plus 0.05% Tween-20 and 5% non-fat dry milk were used to block unspecific sites. The following primary and secondary antibodies were used: ovalbumin polyclonal antibody (ThermoFisher Scientific), Rabbit HRP-conjugated anti-GAPDH (Cell Signalling Technologies, Danvers, MA, USA), Mouse monoclonal anti 6x-His Tag (clone HIS.H8), goat polyclonal anti-CD166, rabbit polyclonal anti-Caveolin 1, rabbit monoclonal anti-Telomerase reverse transcriptase (clone Y182), mouse HRP Conjugate anti β-Actin (clone 8H10D10) were used. Proteins were revealed by GE ImageQuant LAS400 with standard or Femto substrate (ThermoFisher Scientific). Western-blot was quantified by ImageJ software through Analyze>Gels>Label Peaks function and normalized on actin/GAPDH loading controls.

### ELISA

Multiplex or single cytokine (IL-3, CXCL10, CCL5, IFN-γ) ELISA (Thermo Fisher Scientific, MA, USA) were performed on supernatants from OVA-specific T cells co-cultured with cancer cells at ratios 0.5:1, 1:1, 2:1 and 4:1 for 24 hours. Supernatant from T cells cultured alone was used as negative control and the value detracted from the experimental samples.

### Lipid Raft Isolation by Sucrose Gradient Centrifugation, Characterization and Analyses

Lipid rafts were isolated using already described method (Cayrol et al., (2008) Nat immunol 9, 137-145.), employing the detergent 1% Brij58 in TNEV buffer (Hris-HCl, NaCl, EDTA, Na₃VO₄) and sucrose-gradient centrifugation. Cells were scraped in ice cold PBS, counted and lysed with detergent treatment (supplemented with PMSF, protease and phosphatase inhibitor cocktail) 1 hat 4° C. and then homogenized with Potter-Elvehjem. In order to remove unbroken cells, nuclei and cellular debris the homogenate was centrifuged and the supernatant was fractionated in sucrose gradient (42.5%, 35%, 5%) and centrifuged in a SW41 Ti (Beckman Coulter) at 39000 rpm for 19 h at 4° C. A total of ten 1-ml fractions were collected from the top of the gradient to the end of the tube. Fractions were kept frozen at −20° C. until use. Lipid raft were located at fractions 3-4. The concentration of proteins, cholesterol and phosphoplipids in each fraction were measured by a BCA protein assay kit, a cholesterol assay kit and a phospholipids B kit. Inmunoblot analysis was used for specific protein detection: 30 μl of each fraction were diluted with sample buffer, run on NuPAGE™ 10%, Bis-Tris, 1.0 mm, Midi Protein Gels and transferred onto PVDF membranes. Membranes were sequentially incubated with primary antibodies overnight at 4° C. and the day after with horseradish peroxidase-conjugated secondary antibodies. Bound antibody complexes were detected by ECL.

### Immunoprecipitation

FC1242-CLDN18-His and FC1242 cells were solubilized in extraction RIPA buffer and then subjected to BCA protein quantification for the immunoprecipitation of CLDN18.

Clarified cell lysates were incubated with an anti-His antibody or an IgG mouse antibody with rotation and incubation O.N. at 4° C. Then the samples were recovered with Dynabeads™ Protein G (ThermoFisher Scientific) and bound to the magnetic beads during a short incubation with rotation. The resulting antibody complexes with beads were washed 3 times with PBS prior to immunoblot analysis.

### Live Cell Imaging of In Vitro Co-Cultures and Analysis

OVA-specific CTLs cells were co-cultured on the day of imaging with FC1199-OVA CLDN18^{+/+} and CLDN18-cells at a 1:1 ratio. Lymphocytes were allowed to seed for 30 minutes and then activation and dynamics of CTL motility and the susceptibility of PDAC clones to CD8⁺ T cell-mediated cytotoxicity were measured. For T cell activation analysis BioTracker 609 Red Ca2′ AM dye (Merck-Millipore) was used to label OVA-specific CTLs. T cells alone were used as control. For the study of the CD8⁺ T cell dynamics after co-culture with PDAC cells, lymphocytes were labelled with PKH26 red (Merck-Millipore) and their motility behavior was measured every minute for one hour. In addition, to determine CD8+ T cells-induced cytotoxicity, PDAC cells were monitored every 30 minutes for six hours. PDAC cells alone were used as control. For all live imaging experiments, cells were resuspended in phenol-red free medium. Images were acquired with a 40× Plan apochromatic objective (NA 0.6) mounted on an Axio Observer.Z1/7 inverted wide-field microscope (Carl Zeiss Microscopy) equipped with a thermostatic chamber. The environmental conditions were kept at 37° C. in the presence of 5% CO₂. Exposure time for bright field and fluorescence channels was automatically set and left unchanged for the entire duration of the experiment. The multi-field acquisition was achieved using the Tiles tool of Zen v3.5 software (Carl Zeiss Microscopy). The focus plane for each region of interest was automatically set at every time point using the Autofocus tool of Zen v3.5 software (Carl Zeiss Microscopy). For the analysis of T cell motility, individual image acquisitions were converted to movie format and analyzed using Imaris software (Bitplane). Cell tracks were automatically computed using the Spot tool of Imaris software. Only CD8′ T cells in contact with PDAC cells were considered for the analysis. The following motility parameters were calculated: mean velocity (μm/s) displayed by each cell along its path, the arrest coefficient (determined as the proportion of time in which the cell is not moving) and the duration of stable contacts (min) between the OVA-specific CTLs and the FC1199-OVA CLDN18^{+/+} and CLDN18−/− cells. The susceptibility of tumor cells to OVA-specific CTLs-induced cytotoxicity was assessed with the Confluency BioApp of Zen v3.5 software (Carl Zeiss Microscopy), by measuring the reduction over time of the area (μm²) covered by PDAC cells. Particularly, the GFP⁺ area was automatically calculated for every time point of each movie and the values of area computed for each time point were normalized on the area covered by PDAC cells at the initial time point, defined as T0. Accordingly, data were expressed and plotted as the percentage of GFP⁺ area compared to TO over time.

### Intravital Tumor Imaging Preparation

OVA-specific CTLs were labeled for 45 min at 37° C. with 40 mM 7-amino-4-chloromethylcoumarin (CMAC) (Invitrogen -ThermoFisher Scientific) and 2×10⁷ of CTLs were transferred by i.v. injection in immunocompromised Rag2 KO (B6.Cg-Rag2tml.1Cgn/J) mice bearing FC1199-OVA CLDN18^{+/+} and CLDN18/tumor cells when the tumor reach 10 mm×10 mm of volume. Forty-eight hours later, mice were anesthetized by intraperitoneal (i.p.) injection of ketamine (100 mg/kg body weight)/xylazine (15 mg/kg) solution and prepared for the surgery. Hair on back was shaved, skin was sterilized with 70% ethanol and an incision was made to expose the tumor mass. The anesthetized mouse was fitted on a customized microscope stage after microsurgery, minimizing respiratory-induced movements.

### Two-Photon Imaging Acquisition and Data Analysis

Imaging was performed on a customized upright Leica TCS SP5 AOBS confocal-multiphoton system using a thermostatic blanket system to maintain mouse temperature at 37° C. CMAC-labeled OVA-specific CTLs and GFP⁺ tumor cells were excited with a mode-locked Ti:Sapphire Chameleon Ultra II laser (Coherent Inc) and visualized with an Olympus XLUMPlanFI 20×/0.95 water immersion objective. Fluorescence emission from the two different fluorescent dyes was separated through panchromatic electronic barrier filters and detected as green (490-560 nm), and blue (400-500 nm) signal. Stacks of images were acquired using the Leica acquisition software. To create time-lapse sequences, we scanned volumes of tissue, typically 75-100 μm of z-stacks, sampling every 2 μm. Image stacks were acquired at 30 s time intervals for 30-60 min movies. Multidimensional rendering was performed with Imaris (Bitplane). Raw image files were imported into the imaris software(Bitplane) to be transformed into volume-rendered three-dimensional movies, and cell movement analysis was performed. The three-dimensional spatial position of each cell was detected based on centroid fluorescence intensity. Tracking and segmentation of lymphocytes were performed with Imaris Spots function in which tracks from each cell consisting of serial sets of xyz coordinates of single cell centroid. Once the cell tracks have been created on a robust number of cells several parameters can be used to describe the migration dynamics of cells quantitatively as described in (Dusi et al., (2019) Front Immunol 10, 2436; Zenaro et al., (2013) Immunol Cell Biol 91, 271-280).

### Histopathology Analysis and Digital Image Analysis

Tumor samples were fixed after mice sacrifice and organs dissection in 10% neutral buffered formalin and embedded into paraffin. For immunohistochemistry of paraffin-embedded samples, 3 μm thick slides were deparaffinized, serially rehydrated and, after the appropriate antigen retrieval procedure, primary antibodies were added for overnight staining at 4° C. (alternative: 1 h, at room temperature). MID (Thermo Fisher Scientific/Invitrogen) was used for CLDN18 staining at concentration 0.2 μg/ml overnight (alternative: 1.5 μg/ml, 1 h, at room temperature). Abcam anti-CD3 antibody (ab16669), clone SP7 was used for lymphocyte staining at working dilution 1:500, 1 h, at room temperature or overnight staining at 4° C. Secondary antibodies were incubated for 30 minutes at room temperature (Power-Vision HRP anti-Rabbit for CLDN18 & CD3 staining). Immunoreactive antigens were detected using streptavidin peroxidase Vector NovaRED (Vector Laboratories) for CD3 staining and Permanent HRP Green (Zytomed) for CLDN18 staining. After chromogen incubation, slides were counterstained in Hematoxylin (Thermo Fisher Scientific) and brightfield images were scanned with Whole Slide Scanner (Axio Scan.Z1 (Carl Zeiss Microscopy) & NanoZoomer S360 (Hamamatsu Photonics). Digital Image Analysis and cell quantification was done with Tissue Studio (Definiens) and HALO software (Indica Labs). For the first xenograft models FC1199, FC1242 and DANG, CLDN18 expression (CLDN18⁺ tumor tissue area), CD3 amount, and localization was detected by Tissue Studio (Definiens) and visualized in a Heatmap illustration. As follow up DANG-CLDN18 analysis on 11 new xenografts was performed with HALO software (adapted algorithm: Immune Cell v1.3) to separate Tumor tissue, Necrosis, and capsule by classifier. For the cell-based Analysis Claudin 18 expression, CD3 amount, and localization were determined and visualized in a tumor core analysis based on the mid of each tumor (500 μm bins). For the human PDAC cohort study, 148 cases in 8 TMA slides were double stained for CD3 lymphocytes and CLDN18 expression. For each CLDN18⁺ TMA-Spot the localization of CD3+ lymphocyte was defined. The distance between CD3⁺ immune cells and their next CLDN18′ tumor cell was measured. The distance range was split in 11 bins (10× bins for 0-100 μm and 1 bin 100 μm<X). For the CLDN18′ spots the Immune Cell Distance was detected. The Analysis was done with HALO and Spotfire software.

### Immunofluorescence and Digital Image Analysis

Tumor samples were fixed in 10% neutral buffered formalin and embedded into paraffin. For immunofluorescence of paraffin-embedded samples, 3 μm thick slides were deparaffinized and serially rehydrated semi-automated in Ventana Discovery XT Stainer (Roche). After the appropriate antigen retrieval procedure, primary antibodies were added serially for 60 min staining at 37° C.

On human pancreatic tumor tissue Ganymed anti-CLDN18 antibody, clone 43-14A at concentration 0.25 μg/ml was used for CLDN18 staining. Roche Ventana anti-CD4 antibody, clone SP35 (ready to use), anti-CD3 antibody, clone 2GV6 (ready to use) and Novus Biologicals, clone SP16 (1:100), were used for lymphocyte staining. Secondary antibodies were incubated for 32 minutes at 37° C. (OmniMap anti-Rabbit HRP for CD4, CD8 and CD3 staining and OmniMap anti-Mouse HRP for Claudin 18 staining (Roche Ventana). Immunoreactive antigens were detected using Opal Fluorophore (Akoya Biosciences). CD4 -Opal 520, CD8-Opal 570, CD3-Opal 620, CLDN18-Opal 690. On murine lung adenocarcinoma model Thermo Fisher Scientific anti-CLDN18 antibody, clone 34H14L15 (Cat. 700178) at concentration 0.1 μg/ml was used for CLDN18 staining. Sino Biological anti-CD4 antibody, clone 766 (1:200) and Thermo Fisher Scientific anti-CD8a antibody, clone 4SM15 at concentration 5 μg/ml were used for lymphocyte staining. Secondary antibodies were incubated for 32 minutes at 37° C. (OmniMap anti-Rabbit HRP for CD4 and Claudin 18 staining and OmniMap anti-Rat HRP for CD8a staining (Roche Ventana). Immunoreactive antigens were detected using Opal Fluorophore (Akoya Biosciences). CD4-Opal 520, CD8a -Opal 570, Claudin 18-Opal 690. After fluorophore incubation (20 min) slides were counterstained with DAPI (Sigma Aldrich).

Immunofluorescence images were scanned with Whole Slide Scanner (Phenolmager (Akoya Bioscience). Digital Image Analysis and cell quantification was done with HALO software (Indica Labs). 11 IF-stained human pancreatic PDAC tumor samples were analyzed with HALO software (adapted HighPlex algorithm). To perform tumor classification, pathologist expertise was employed to draw the classes: Invasive tumor, PanIN 1, PanIN 2, PanIN 3, and normal endocrine pancreas; using HALO Link (Indica Labs). Cell analysis for CLDN18, CD3, CD4, CD8 was performed within the respective annotations to collect values for each tumor stage to subsequently display number and distribution in the density heatmap as phenotypes within 250 μm. For the murine KP lung adenocarcinoma model, annotations were also made using pathologist expertise. Tumor classification by grading (G1, G2, G3) was performed on the HEs of the staged mouse lungs and plotted in HALO Link. The annotations were transferred into the HALO software, tumor size measured (diameter) and a cellular analysis for immune cells (CD4, CD8a) and CLDN18 was performed in HALO software (adapted algorithm: HighPlex FL v4.1.3).

### Cell Line Immunofluorescence and Image Acquisition

FC 1199-OVA CLDN18+/+ or FC 1199-OVA CLDN18−/− cells were grown in chamber slides (Lab-Tek-II-Thermo Fisher Scientific) and fixed with PFA 4% for 10 min prior blocking of unspecific binding sites with PBS-BSA 1% for 2 hours at RT. Staining was done with a polyclonal unconjugated anti-ALCAM antibody at 1:400 (Thermo Fisher Scientific) overnight at 4° C. in PBS-BSA 1%. Then, secondary antibody donkey anti-goat AF546 IgG (Thermo Fisher Scientific) 1:500 in PBS+1% BSA was added for 1 hour at RT and GFP was visualized as a cell co-stain. For colocalization studies of CLDN18 and adhesion molecules FC1199 cells were grown in chamber slide as described above and staining was performed with the primary antibodies anti-CLDN18 (Imab 362) 1:500, anti-ALCAM (Thermo Fisher Scientific) 1:400, anti-ICAM-1 (Biolegend) 1:100 in PBS-BSA 1% O/N at 4° C. Then, a donkey anti-goat AF680 IgG (Thermo Fisher Scientific), a goat anti-human AF405 IgG (Thermo Fisher Scientific) both 1:500 or a goat anti-rat AF405 IgG (Thermo Fisher Scientific) 1:250 in PBS-1% BSA were added for 1 hour each at RT. Gold Prolong Antifade (Thermo Fisher Scientific) were used to mount coverslips (Menzel-Gliser) prior to the visualization of slides.

For ALCAM cell surface expression images were captured on an Axio Imager Z2 microscope (Carl Zeiss Microscopy). Images were acquired with a constant exposure time determined by staining with secondary antibodies alone. Acquired images were quantified on 6 ROI per field, blindly choosen on the GFP channel. Nine fields were acquired for each cell clone and the fluorescence of the ALCAM adhesion molecule was measured by ImageJ using the same threshold. Results were then represented as median fluorescent ALCAM expression normalized to mm². For colocalization study images were captured on a Leica TCS SP5 AOBS confocal equipped with a Plan-Apochromat 63× oil immersion objective. Excitation lasers used were 405 nm and 633 nm and detectors were set according to the corresponding fluorophores used. Typically, images were acquired at 512×512 pixels, 400 Hz scan speed and with the pinhole set to 0.95 Airy unit. At least nine fields were acquired for each well chamber. Raw image files were imported into the Imaris software (Bitplane). A colocalization channel of CLDN18, ALCAM or ICAM-1 on cell surface membrane of the hot cell line was made in Imaris using the Colocalization tool. Surface function was used to segment protein expression and masked them onto separate channels before applying colocalization to avoid background signal.

### Example 1

### Characterization of Hot and Cold Immunogenic Pancreatic Tumors.

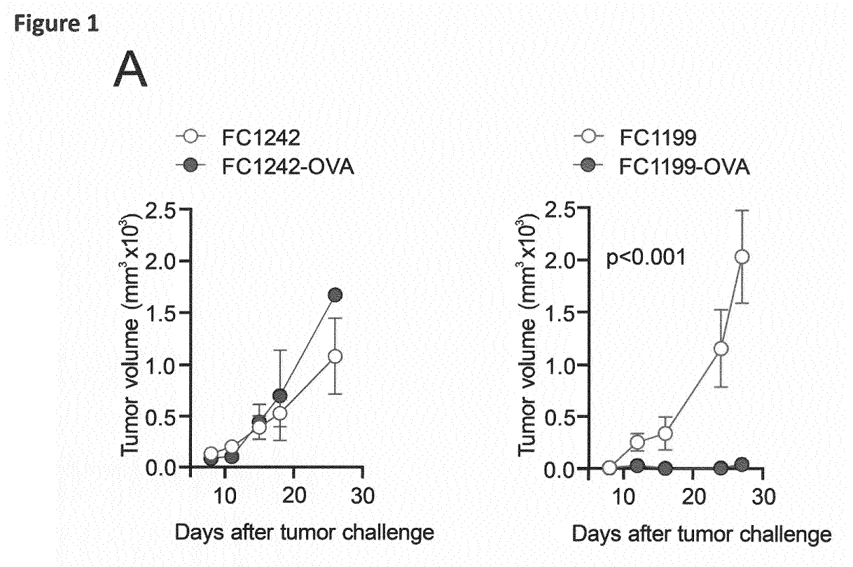

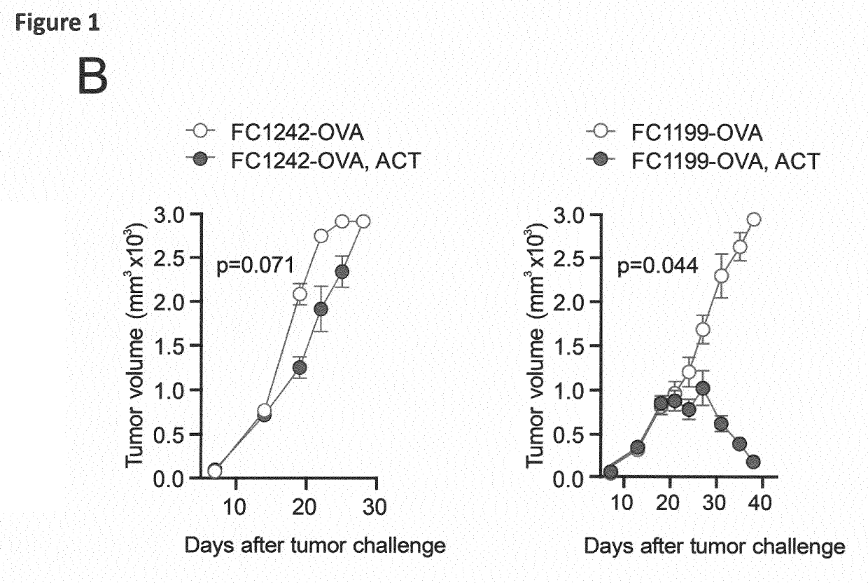

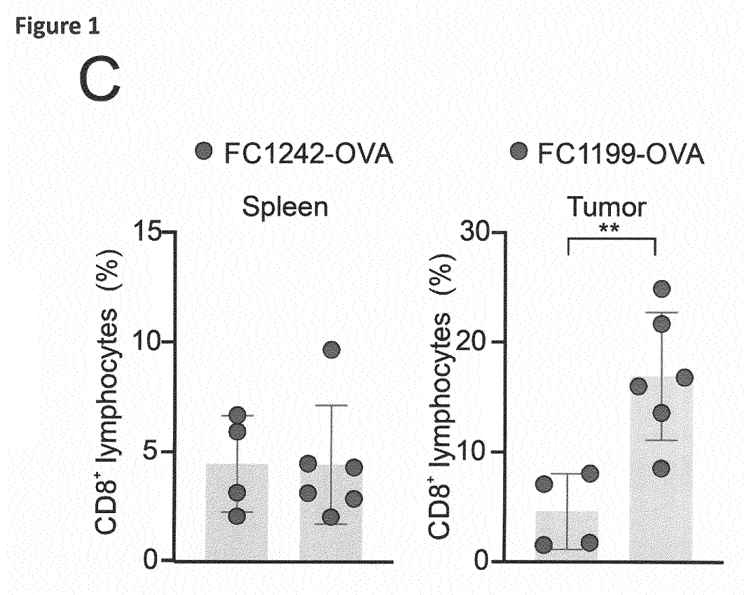

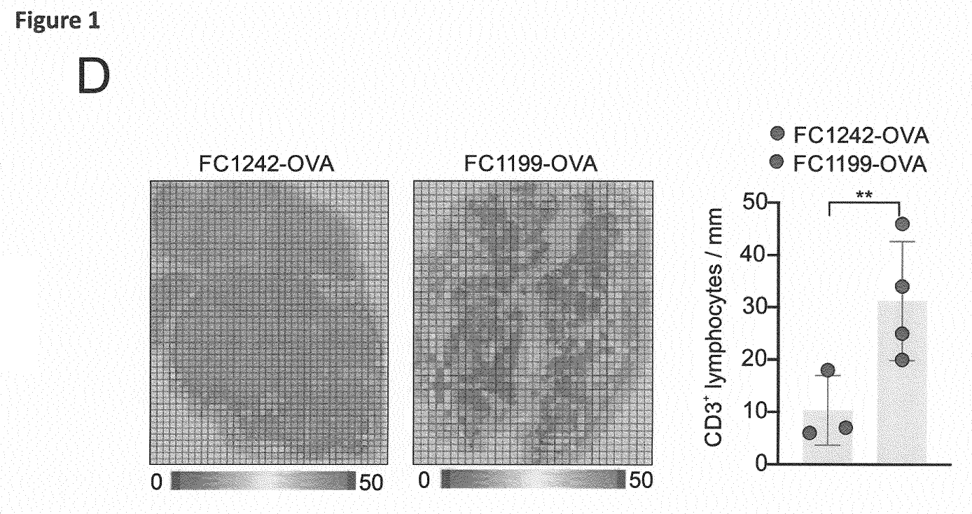

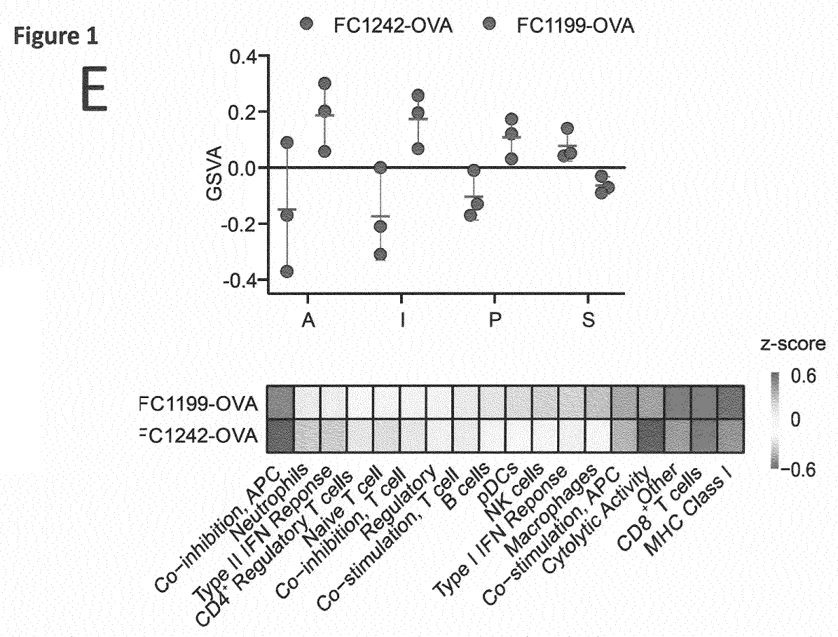

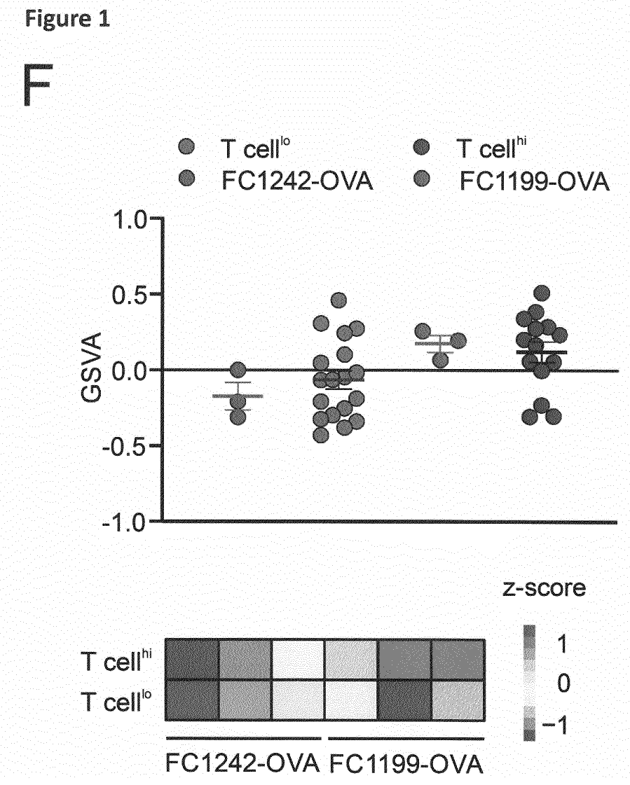

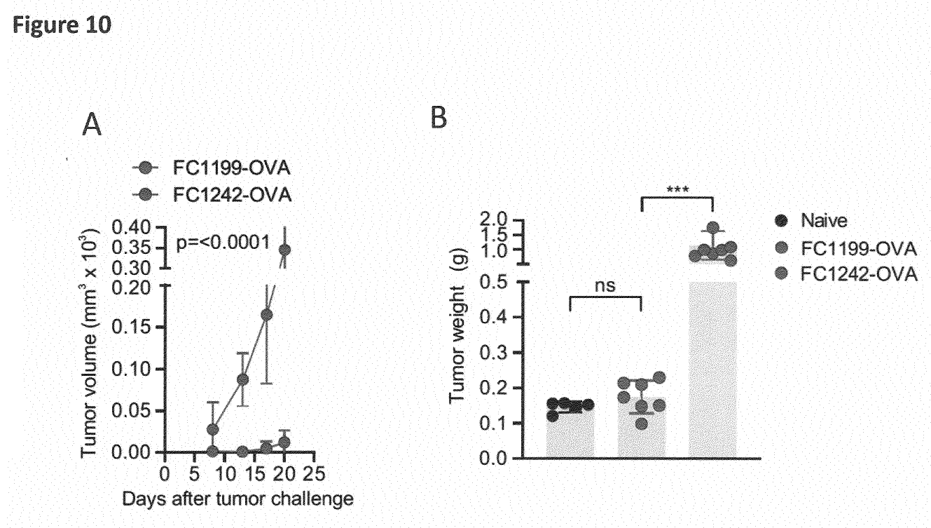

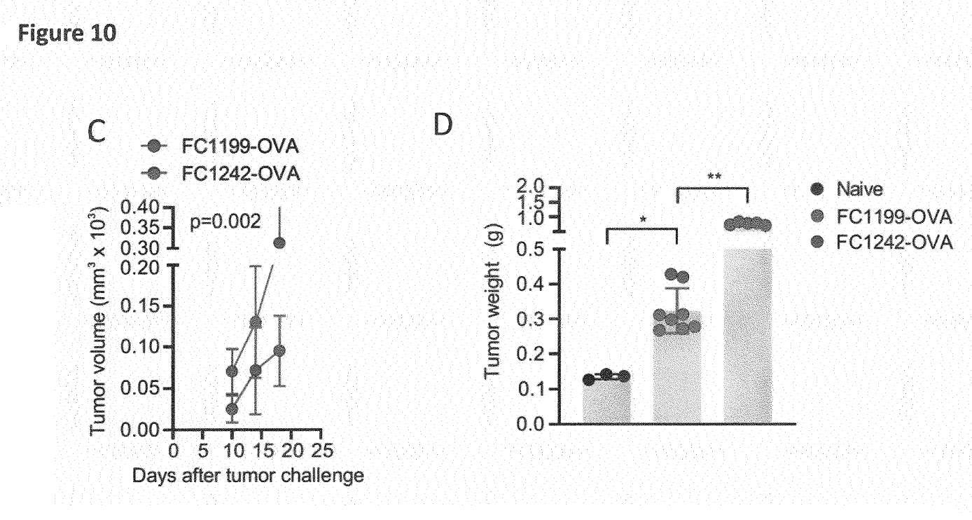

To unveil cancer-driven cues influencing the transition toward either a “hot” or “cold” TME in pancreatic cancer, we initially exploited Kras^{LSL-G12D/+}, Trp53^{LSL-R172H/+}, Pdx1-Cre (KPC) mice (Hingorani, S. R. et al. (2005) Cancer Cell 7, 469-483). Four KPC cell lines were derived, either expressing the endogenous telomerase (TERT) tumor-associated antigen (TAA) alone (FC 1199 and FC1242 cell lines) or engineered to produce a surrogate “strong” tumor antigen (Ovalbumin -OVA; hereafter named FC1199-OVA and FC1242-OVA cells). Established cell lines were characterized for MHC1 and OVA expression, proliferation and ability to be recognized and killed by either OVA-specific or TERT-specific CTLs. Although no relevant differences were detected in vitro, the engineered cell lines were endowed with wholly different features when growing in immune competent C57Bl/6 mice. The FC1242-OVA cell line grew similarly to its parental counterpart, whereas FC1199-OVA development was completely curbed (FIG. 1A), regardless of whether injected either orthotopically (in the pancreas (FIG. 10)) or i.v. to induce lung metastases. Host immune competence was mandatory to allow effective cancer control since the antitumor activity was completely abrogated in immune-compromised mice lacking adaptive immunity (FIG. 10 C-D). The antitumor activity toward FC1199-OVA did not rely on a superior T-cell immune response towards the OVA antigen. Indeed, both FC1242-OVA and FC1199-OVA cells triggered comparable levels of OVA-specific CTLs, as confirmed by IFNγ spot quantification in the spleen of C57Bl/6 mice. We thus evaluated whether heightened tumor control could reflect differential T-cell homing to the tumor in FC1199-OVA-bearing mice. The adoptive cell transfer (ACT) of tumor-specific CTLs in immune-deficient hosts was completely ineffective in FC1242-OVA-bearing mice but successfully restrained tumor progression in FC1199-OVA-bearing mice, improving their survival (FIG. 1B). Accordingly, three days after ACT, we detected more CTLs in FC1199-OVA than FC1242-OVA tumors by FC (FIG. 1C, right panel) and IHC (FIG. 1D) but not in peripheral immune organs such as the spleen (FIG. 1C, left panel), suggesting similar systemic CTL persistence in the two settings. These data indicate that the failure in immune control by adaptive immunity correlated with a deficit in T-cell homing and retention within the tumor. FC1199-OVA and FC1242-OVA cells could thus recapitulate “hot” and “cold” PDACs, respectively.

We then profiled the cold and hot tumors, and the principal component analysis (PCA) revealed distinct molecular signatures in the two cell lines. We further investigated these profiles using the molecular classification previously established to correlate the histological features of human PDACs with overall survival (OS) (Bailey, P. et al. (2016) Nature 531, 47-52). FC1242-OVA showed a fingerprint of the squamous tumor subtype, which is associated with poor prognosis. Conversely, FC1199-OVA showed mixed molecular features shared with the immunogenic, aberrantly differentiated endocrine exocrine (ADEX), and progenitor subtypes of PDAC (FIG. 1E, top panel). The “immunogenic” tumors share many characteristics with progenitors and ADEX but hold a private signature associated with leukocyte infiltration, activation and likely immune suppression (Bailey, P. et al. (2016) Nature 531, 47-52). In agreement with the gene expression profiles, we unveiled biological processes related to immune activation (including antigen presentation, co-stimulation, and cytolytic activity) and downregulation of genes involved in immune suppression (FIG. 1E, bottom panel) in FC1199-OVA cells. We further compared the hot and cold tumor signatures with those decoded from a panel of mouse PDAC lines classified as either T-cell low or high for the presence of either low or high numbers of tumor-infiltrating lymphocytes (TILs) in the TME, respectively (Li, J. et al. (2018) Immunity 49, 178-193 e177). As expected, gene set variation analysis (GSVA) employing both the whole transcriptome (Li, J. et al. (2018) Immunity 49, 178-193 e177) and subtype classifications (Bailey, P. et al. (2016) Nature 531, 47-52) showed that the molecular signatures of the hot and cold tumor cell lines closely resembled those of TiLs^{hi} and TILs^{lo}, respectively (FIG. 1F, Table 1).

TABLE 1
Multivariate analysis (COX) for CLDN18 in TCGA LUAD dataset
TCGA LUAD dataset_COX

| Parameter | OS HR (95% CI) | p |
|---|---|---|
| CLDN18 and CD3E (ref. | | 0.047 |
| CLDN18 low CD3E low) | | |
| CLDN18 high CD3E high | 0.46 (0.24-0.88) | |
| CLDN18 high CD3E low | 1.26 (0.57-2.79) | |
| CLDN18 low CD3 high | 0.71 (0.48-1.063) | |
| Age | | 0.51 |
| Gender | | 0.42 |
| Male vs Female | 1.16 (0.81-1.66) | |
| Residual tumor | | 0.0023 |
| R1 vs R0 | 2.27 (1.08-4.73) | |
| R2 vs R0 | 10.36 (2.20-48.88) | |

### Example 2

### Hot Tumors Share a Cell Adhesion Signature.

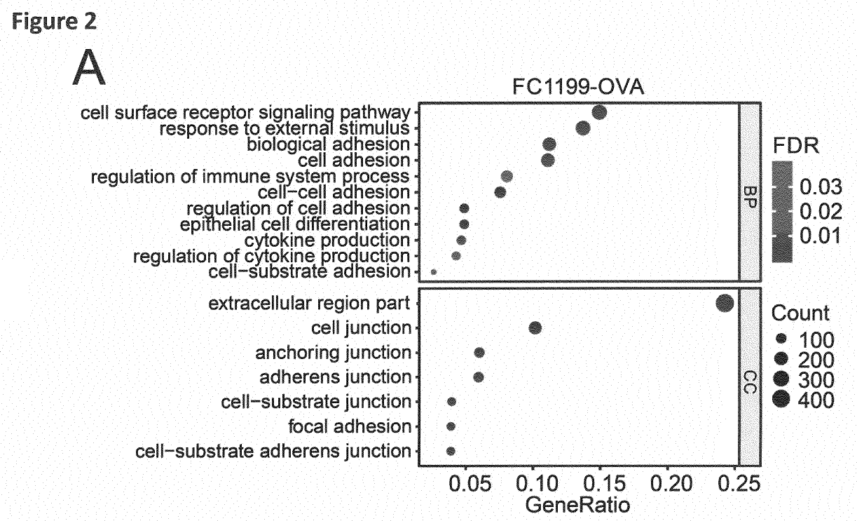

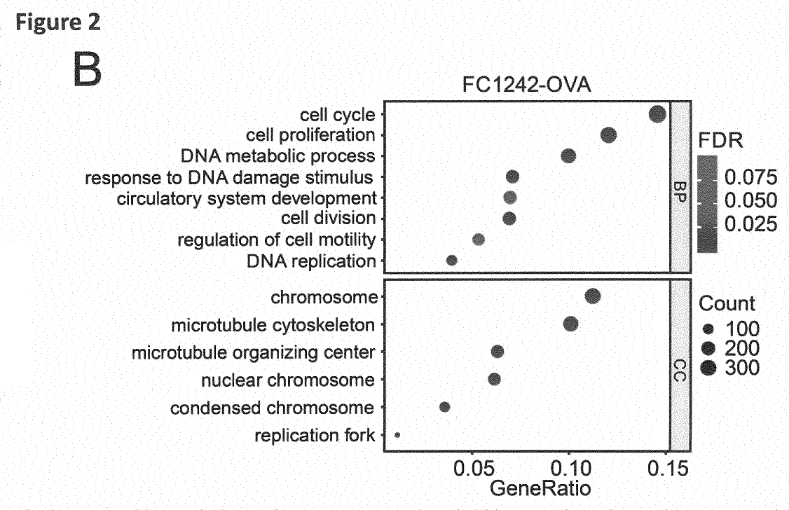

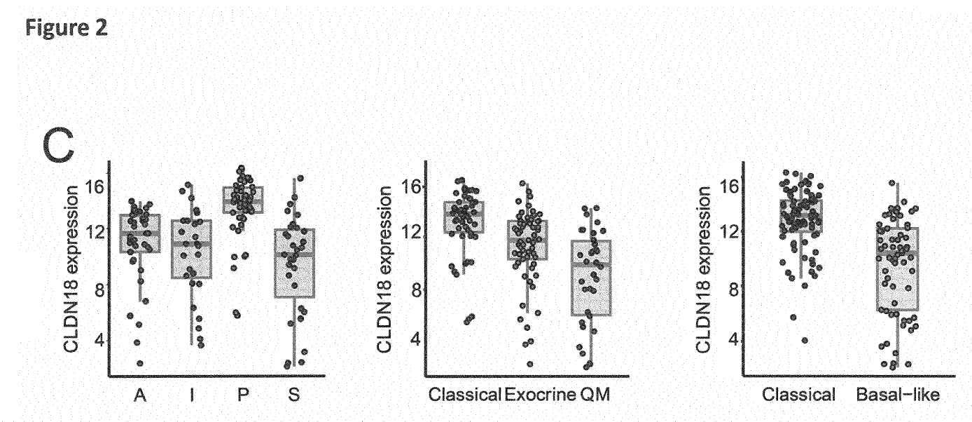

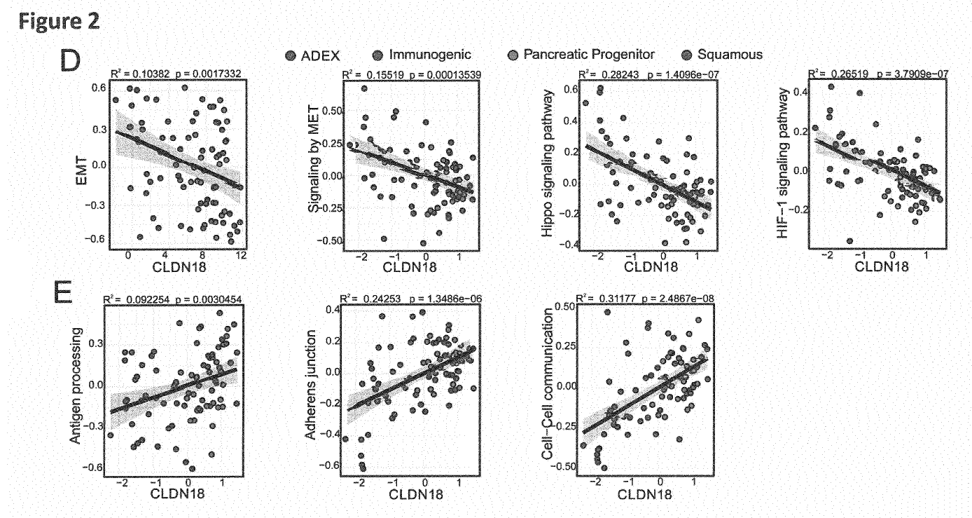

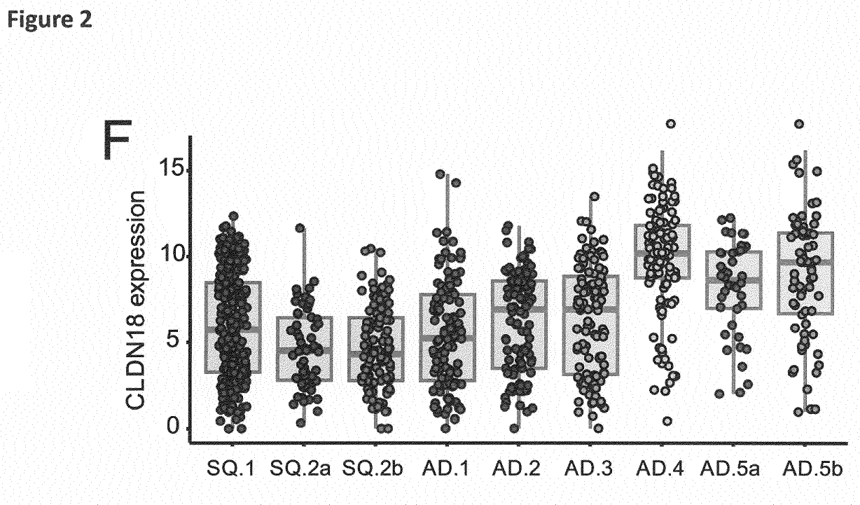

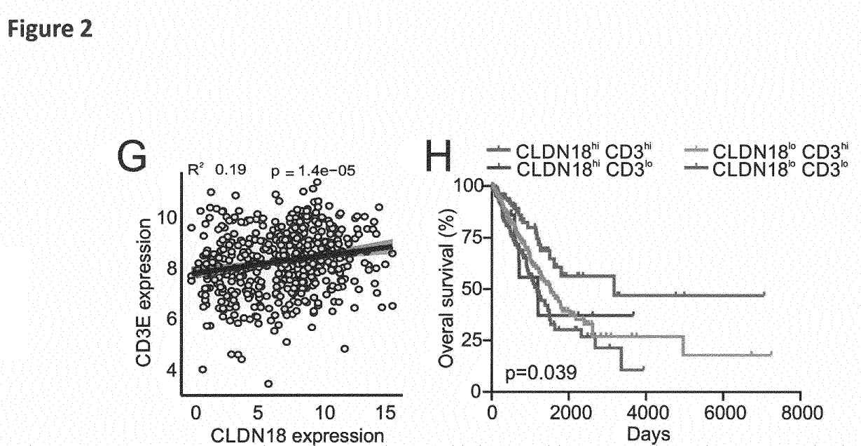

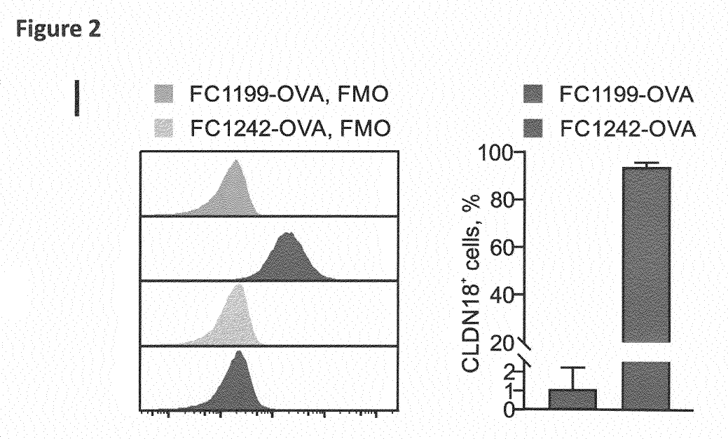

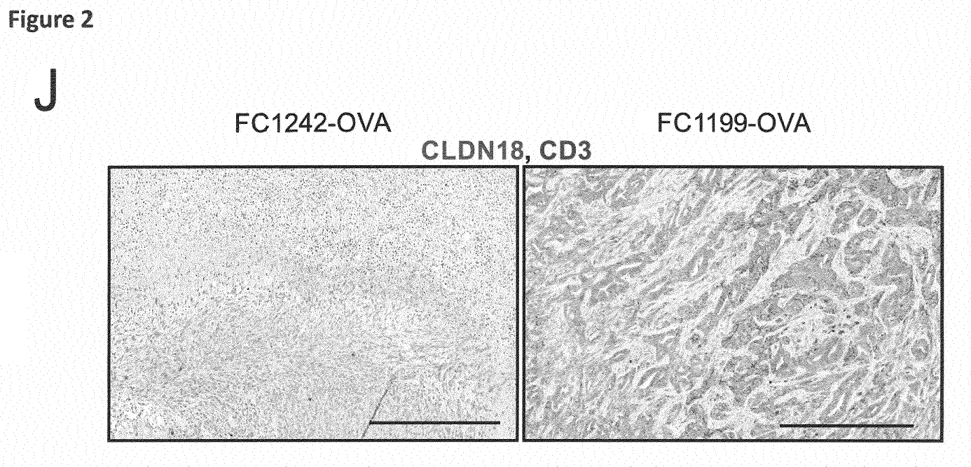

We further characterized cold and hot PDAC tumors to pinpoint gene candidates that might underlie the different levels of T-cell infiltration. Gene ontology analysis revealed a unique signature in the hot (FC1199-OVA) cell line, including the activation of biological processes related to the cell surface receptor signaling pathway, cell adhesion, cytokine production and regulation of immune system processes. Moreover, we identified several membrane proteins coupled with cell anchoring and adherent junctions within the cellular components (FIG. 2A). Conversely, the cold (FC1242-OVA) cell line showed enrichment in biological processes related to cell proliferation, cell division, DNA replication, as well as DNA and nuclear organization (FIG. 2B). Genes upregulated in cold tumors were related to stemness (Quan, M. Y. et al. (2020) Front Cell Dev Biol 8, 287), resistance to chemotherapy (Luo, L. et al. (2021) Cell Death Dis 12, 169), immune suppression and tumor progression (Blasco, M. T. et al. (2019) Cancer Cell 35, 573-587 e576; Principe, D. R. et al. (2016) Cancer Res 76, 2525-2539), including Sox2, Bcat1, Fgfrl, Egfr, and Tgfb2. Within the biological adhesion process category, a number of receptors emerged with cell-cell interaction properties, including adhesion molecules, integrins, and a family of proteins participating in tight junction assembly and maintenance, i.e., claudins. Within this family, CLDN18 was the most upregulated. We verified whether CLDN18 expression was associated with explicit pancreatic cancer features and could identify molecular PDAC subtypes with prognostic and biological relevance. By comparing acknowledged molecular classifications of pancreatic cancers (Bailey, P. et al. (2016) Nature 531, 47-52; Collisson, E. A. et al. (2011) Nat Med 17, 500-503; Moffitt, R. A. et al. (2015) Nat Genet 47, 1168-1178) within the pancreatic adenocarcinoma (PAAD) TCGA dataset (Cancer Genome Atlas Research, N. (2017) Cancer Cell 32, 185-203 el13), we uncovered a CLDN18 association with classical, progenitor ADEX, immunogenic PDAC subtypes, which are related to more differentiated tumors, presence of immune infiltrate and better overall survival compared to squamous (S), quasi mesenchymal (QM) and basal-like subtypes in which CLDN18 expression is lower (FIG. 2C). By investigating the molecular dataset from the International Cancer Genome Consortium (ICGC) (Bailey, P. et al. (2016) Nature 531, 47-52), we also identified a significant inverse correlation between CLDN18 and molecular processes associated with neoplastic progression, including epithelial to mesenchymal transition (EMT), as well as c-MET, Hippo and hypoxia-inducible factor 1 (HIF-1) signaling pathways (FIG. 2D). Furthermore, we observed a positive correlation between CLDN18 expression and the regulation of tissue architecture, cell-cell communication and immune activation (FIG. 2E). We explored whether CLDN18 could be relevant in defining the molecular subtype, immune landscape and clinical outcomes in other solid tumors. By investigating the available TCGA datasets of non-small cell lung cancer (NSCLC) with established molecular classification (Chen, F. et al. (2017) Oncogene 36, 1384-1393), we uncovered preferential CLDN18 expression in lung adenocarcinoma rather than squamous carcinoma (FIG. 2F). Within LUAD, CLDN18 expression was higher in the AD.4 and AD.5 subtypes, which are both associated with high immune cell infiltration and better OS (Chen, F. et al. (2017) Oncogene 36, 1384-1393). Notably, we identified a significant positive correlation between CLDN18 expression and both CD3+ and CD8⁺ T lymphocytes (FIG. 2G) in LUAD patients. Accordingly, CLDN18 expression cooperates with TIL presence within the tumor to stratify and identify LUAD patients with improved OS (FIG. 2H).

### Example 3

### CLDN18 Ablation Impairs Tumor Hotness.

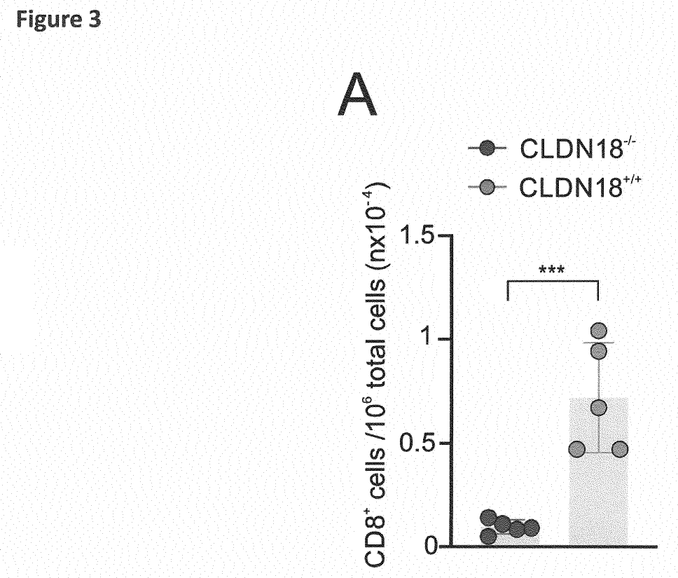

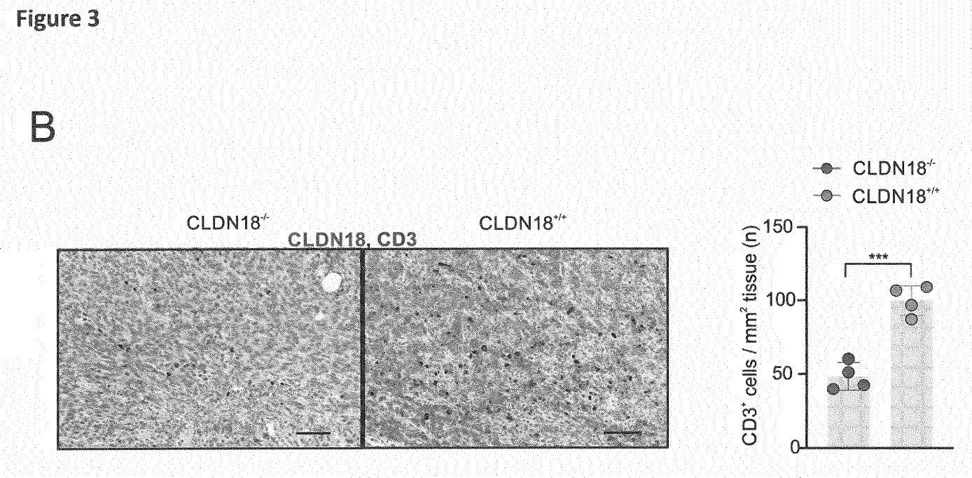

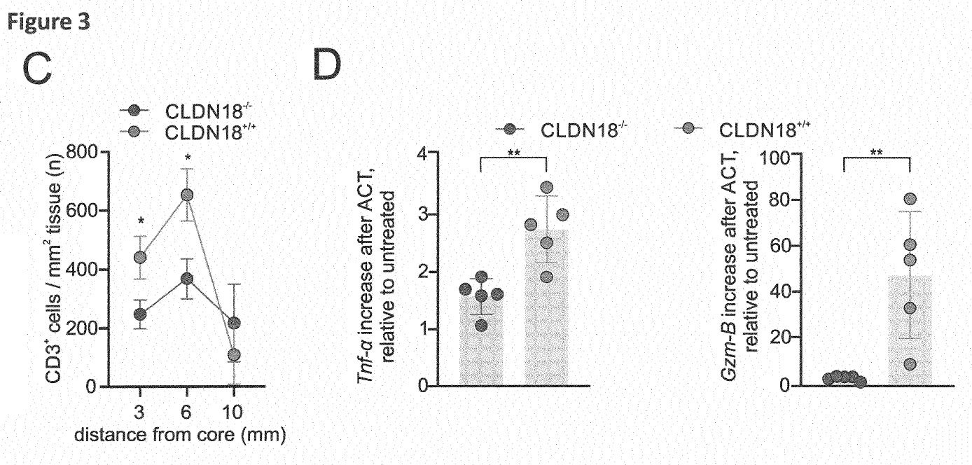

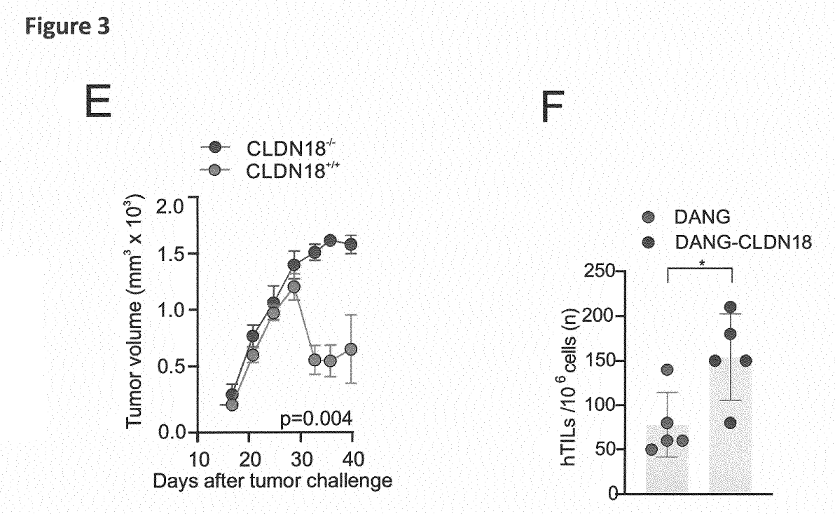

We questioned whether CLDN18 could directly contribute to orchestrating CTL infiltration. By both FC and IHC, we confirmed that CLDN18 was highly expressed only in hot tumors (FIG. 2I-J). Next, we deleted the Cldn18 gene by CRISPR-Cas9 technology in the FC1199-OVA cell line and generated a number of clones lacking the protein, as confirmed by FC and Sanger sequencing. After confirming similar expression of OVA protein, we injected CLDN18^{-/+} and CLDN18^{-/-} clones into immune-deficient mice. CLDN18 ablation significantly impaired the in vivo tumor infiltration of adoptively transferred, OVA-specific CTLs, as shown by both FC (FIG. 3A) and IHC (FIG. 3B). We employed an “earthquake” analysis to track T-cell positioning from the center to the periphery of tumor tissues, and found that CLDN18 supported the overall T-cell infiltration since its ablation significantly impaired TIL presence, especially in close proximity to the inner tumor core (FIG. 3C). CLDN18-dependent T lymphocyte infiltration was coupled with increased expression of genes associated with T lymphocyte activation and killing in the tumor tissue, including tumor necrosis factor-α (Tnf-α) and granzyme-B (Gzm-B) (FIG. 3D). Accordingly, although CLDN18^{+/+} and CLDN18^{-/-} tumors progressed in a comparable manner in untreated groups, increased T lymphocyte homing to CLDN18^{+/+} tumors resulted in heightened tumor control by ACT (FIG. 3E).

We then extended the initial findings to human PDAC tumors. To this aim, we employed human HLA-A2⁺ PDAC cell lines expressing human telomerase either negative or positive for CLDN18 (DANG and DANG-CLDN18, respectively), and TCR-engineered human T lymphocytes recognizing hTERT with high avidity (Sandri, S. et al. (2016) Cancer Res 76, 2540-2551; Sandri, S. et al. (2017) Oncotarget 8, 86987-87001). We injected either DANG or DANG-CLDN18 cells in opposite flanks of immune-deficient NOD Cg-Prkdcscid Il2rgtmlSug/JicTac (NOG) mice. When tumors were established, we i.v. transferred TERT-specific CTLs and collected tumors to identify TILs. As expected, T lymphocytes preferentially homed to CLDN18⁺ tumors, as shown by both FC (FIG. 3F) and IHC (FIG. 3G). Notably, T-cell infiltration was coupled with significantly improved control of tumor progression in CLDN18⁺ tumors (FIG. 3H).

We recapitulated tridimensional tumor architecture in vitro by establishing T lymphocyte-organoid co-cultures in CLDN18⁺ and CLDN18-tumors. CLDN18 presence sustained T-cell adhesion to tumor organoids, as shown by T-cell quantification and IF imaging. Since CLDN18 regulates H⁺ fluxes and orchestrates the acidic pH microenvironment in the stomach and given that acidic pH can epigenetically rewire T cell stemness and cytotoxic abilities (Cheng et al., (2023) Nat Metab 5, 314-330; Feng et al., (2022) Nat Commun 13, 4981), we sought to explore whether results were affected by diverse tumor pH. However, we could not identify any significant difference in tumor pH in vivo in CLDN18-deficient and CLDN18-proficient tumors. Moreover, we also confirmed that CTL recruitment to tumors was not influenced by soluble chemo attractants upregulated in a CLDN18-dependent fashion. Indeed, DANG-CLDN18 cell conditioned media did not affect T lymphocyte migration more than the CLDN18⁻ negative line, which was true for both activated and resting T lymphocytes.

### Example 4

CLDN18 Supports T-Cell Interaction with Tumor Cells and Sustains T Lymphocyte Activation.

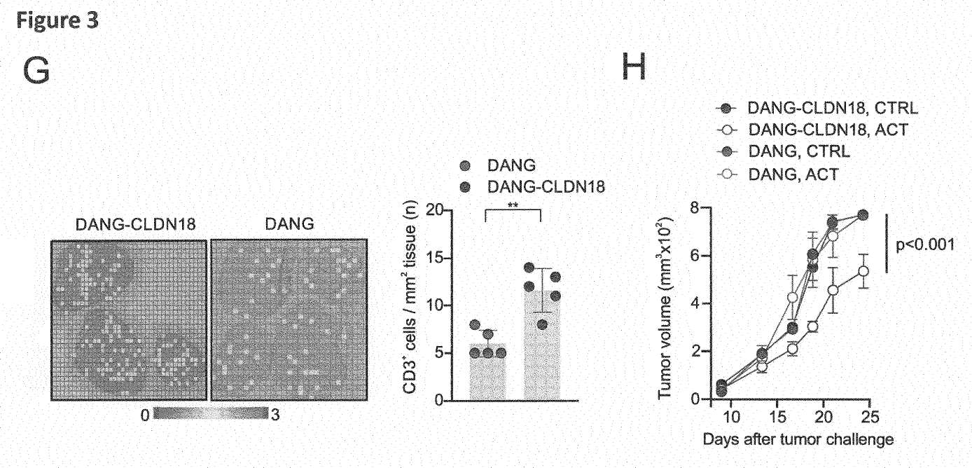

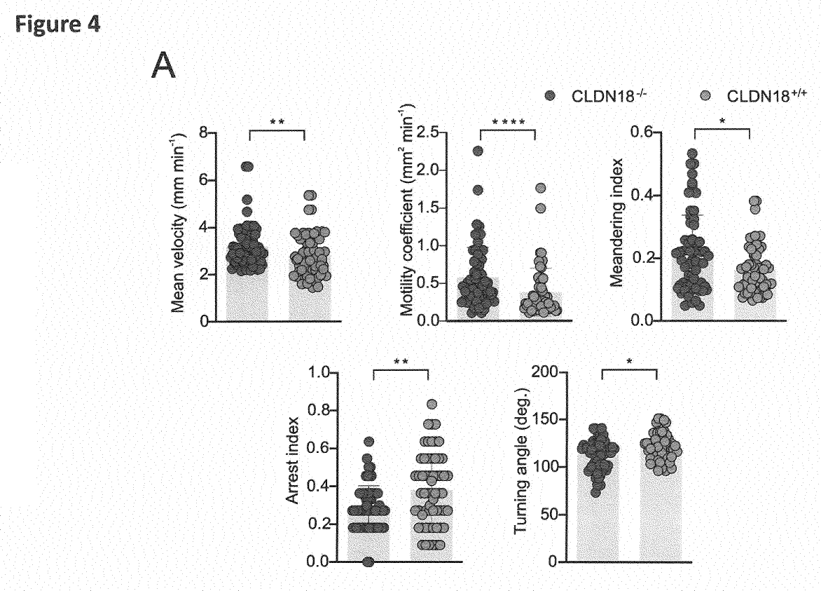

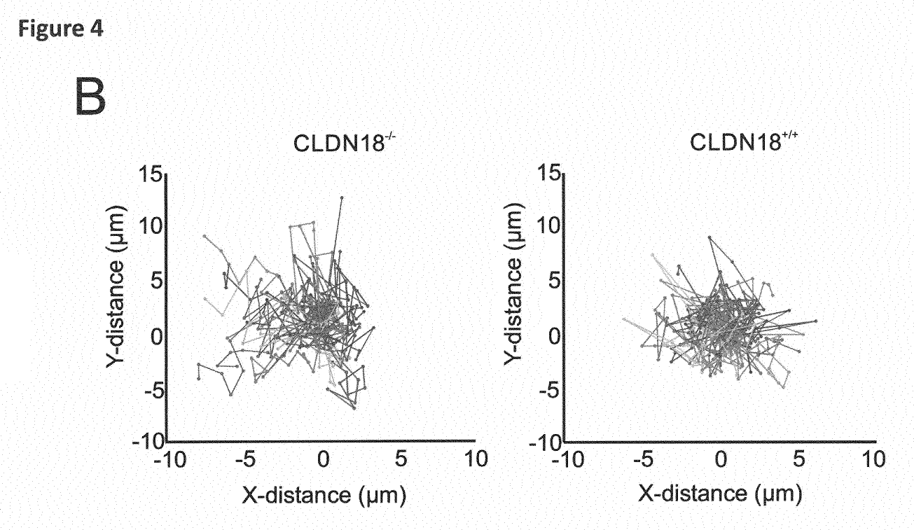

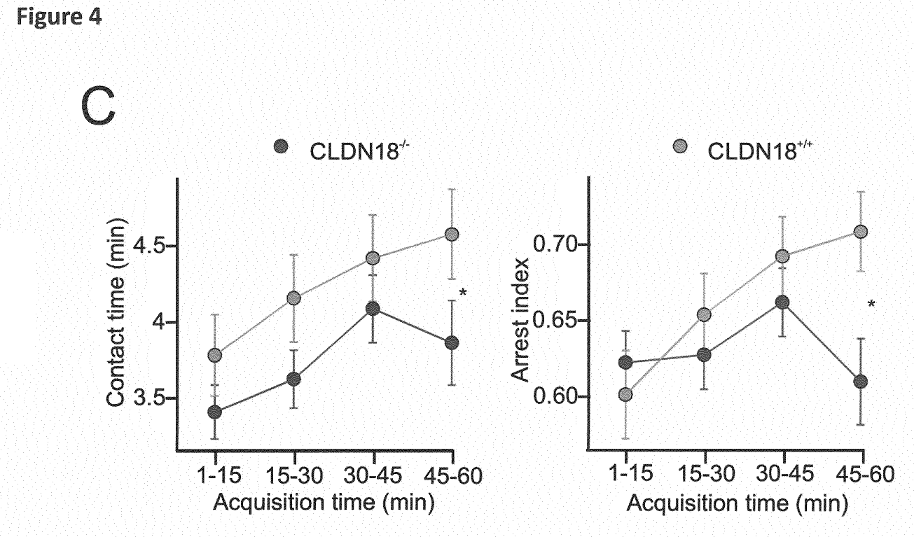

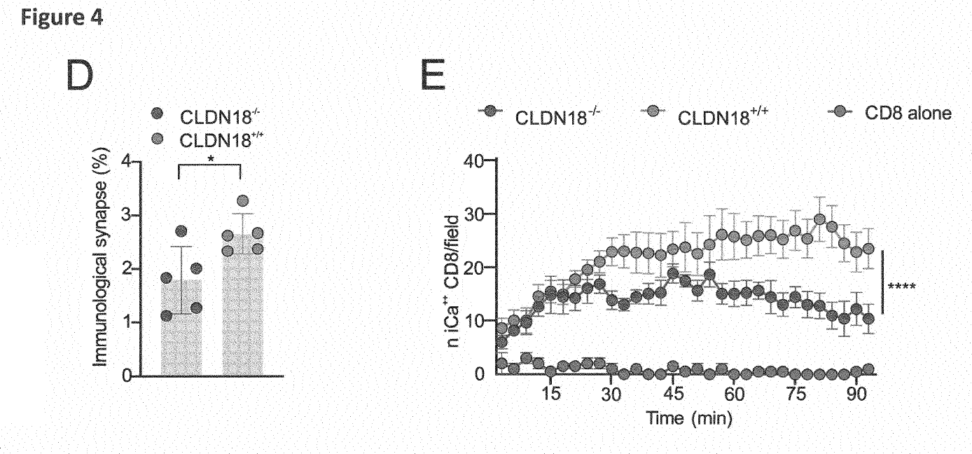

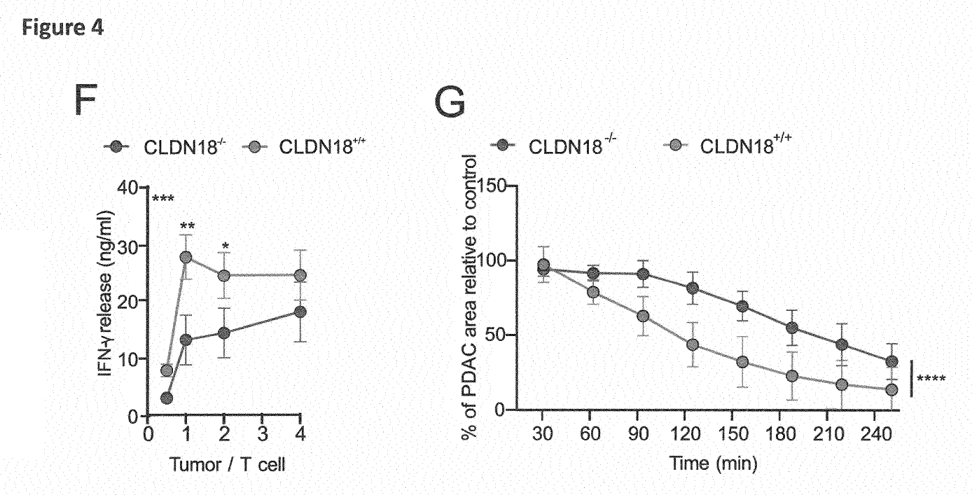

To shed light on the contribution of CLDN18 in regulating T lymphocyte-tumor cell interactions, we followed the intra-tumoral movements of tumor-specific CTLs in CLDN18-proficient and CLDN18-deficient contexts. CLDN18 ablation was associated with looser interactions between T lymphocytes and tumor cells, which resulted in increased T lymphocyte mean velocity, higher motility coefficient and meandering index with a reduced arrest index and turning angle (FIG. 4A). Altogether, CTLs showed wider and faster movement trajectories in CLDN18^{-/-} than CLDN18^{+/+} tumors, suggesting that CLDN18 has a role in strengthening T lymphocyte-tumor cell interactions (FIG. 4B). In vitro evaluation of the CTL motility behaviors in terms of contact time and arrest index in time lapse confirmed stronger interactions of T lymphocytes in the presence of CLDN18 on tumor cell surface (FIG. 4C). We then investigated the functional consequences of CLDN18-dependent interactions by assessing T lymphocyte immunological synapse (IS) formation with CLDN18^{+/+} and CLDN18′ tumor cells, and identified a preferential IS formation toward CLDN18^{+/+} target cells (FIG. 4D). We corroborated these results by monitoring over time the intracellular release of calcium in CTLs as a proxy signal of TCR-mediated T-cell activation (Joseph, N. et al. (2014) Biochim Biophys Acta 1838, 557-568), within the context of CLDN18-proficient and CLDN18-deficient tumors, demonstrating a significant higher IS formation rate in CTLs co-cultured with CLDN18^{+/+} cells (FIG. 4E). We evaluated whether higher frequency of IS formation in CLDN18-proficient tumor context was associated with increased release of cytokines supporting the homing and function of CTLs in the tumor core by multiplex ELISA. We found that increased CTL activation following co-culture with CLDN18^{+/+}, tumor cells resulted in enhanced release of IFNγ (FIG. 4F), IL3, CXCL10, and CCL5 cytokines and improved cytotoxicity (FIG. 4G). Notably, CLDN18-dependent interaction and activation of T lymphocytes did not depend on a different antigen processing and presentation ability since similar results were achieved by pulsing CLDN18-positive and CLDN18-negative clones with the “SIINFEKL” OVA peptide.

### Example 5

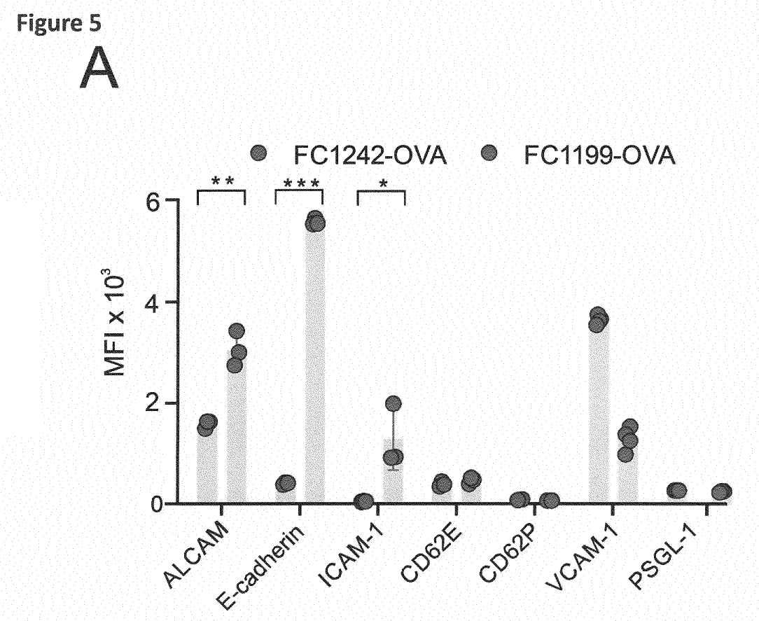

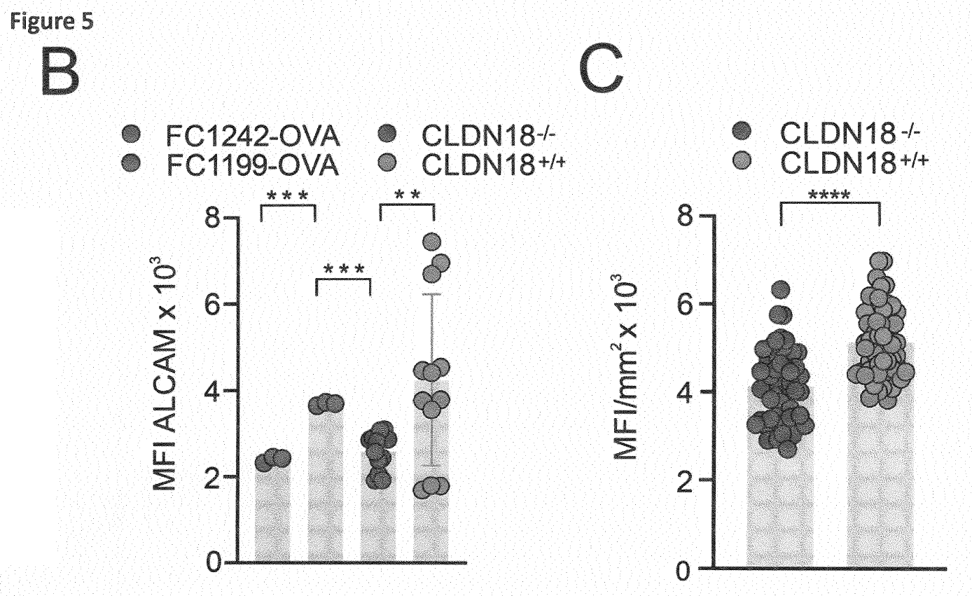

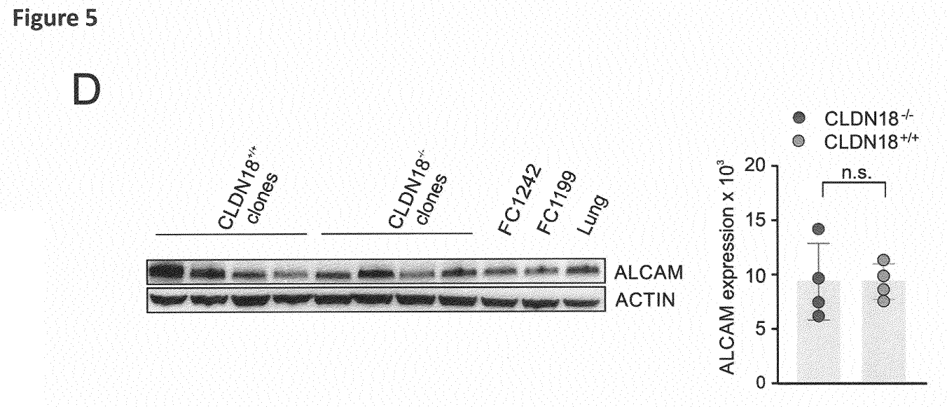

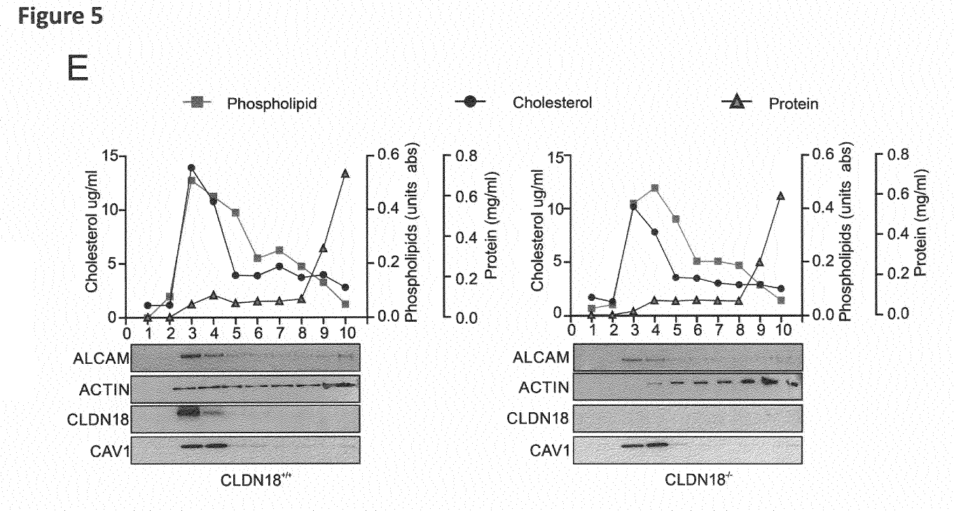

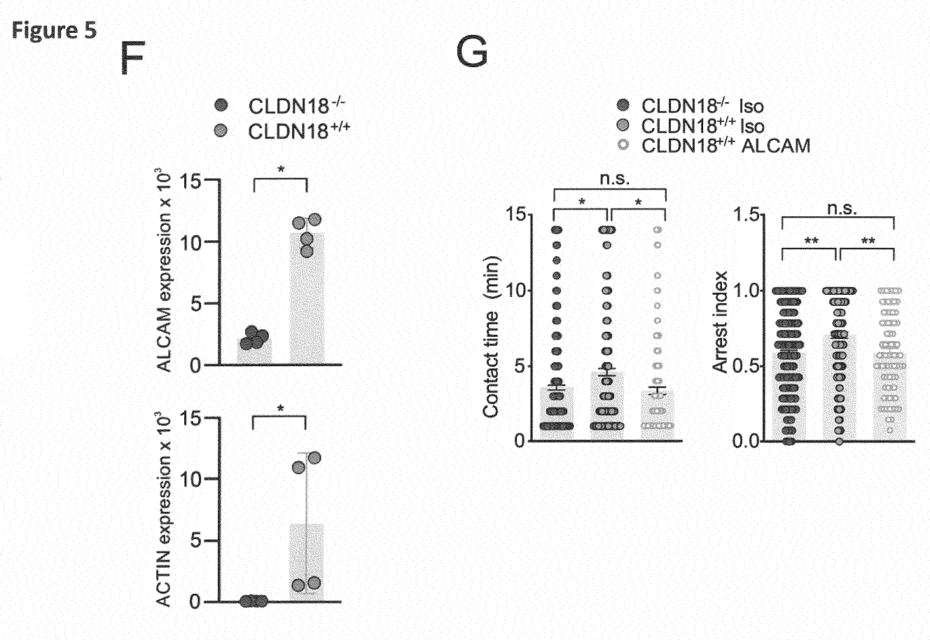

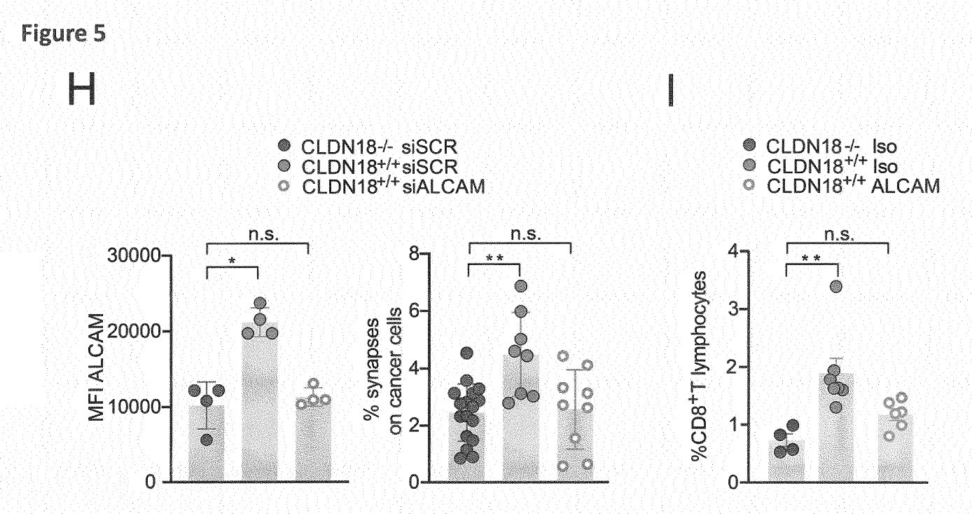

CLDN18 Couples ALCAM Localization in Lipid Rafts and its Interaction with CTLs to Form the IS. CLDNs are proteins mainly expressed by epithelial cells and involved in homotypic and heterotypic interactions between elements of the same family (Krause, G. et al. (2008). Biochim Biophys Acta 1778, 631-645). We found that CLDN18 is absent in either resting or activated CTLs, and we thus speculated that CLDN18 could stabilize T lymphocyte-tumor interactions by regulating the expression, accessibility or membrane accrual of other adhesion molecules on tumor cells. FC analysis showed that the hot cell line expressed higher levels of ALCAM, e-cadherin and ICAM-1 on the membrane than the cold tumor cell line (FIG. 5A). Among these adhesion molecules, only ALCAM was differentially expressed between FC1199-OVA CLDN18^{+/+} and CLDN18^{-/-} tumor cell membranes, as demonstrated by FC extracellular staining and confirmed by surface immunofluorescence staining (FIG. 5B-C). Molecular (RNA-seq, not shown) and proteomic data (FIG. 5D) did not reveal differences in total ALCAM expression among hot and cold tumor clones, pointing out a role for CLDN18 protein in regulating ALCAM distribution on the cell membrane. We further performed confocal microscopy analysis of CLDN18, ALCAM, and other adhesion molecules in the hot tumor cells and confirmed a better CLDN18 co-localization with ALCAM than ICAM-1 on the cell membrane, with a good correlation coefficient. Since ALCAM supports IS formation and downstream CTL activation by binding to its cognate receptor CD6 on CD8+ T lymphocytes (Zimmerman, A. W. et al. (2006) Blood 107, 3212-3220), we hypothesized that CLDN18 could improve T-cell activation by driving ALCAM preferential recruitment to functional membrane domains, i.e. the lipid rafts, in tumor cells.

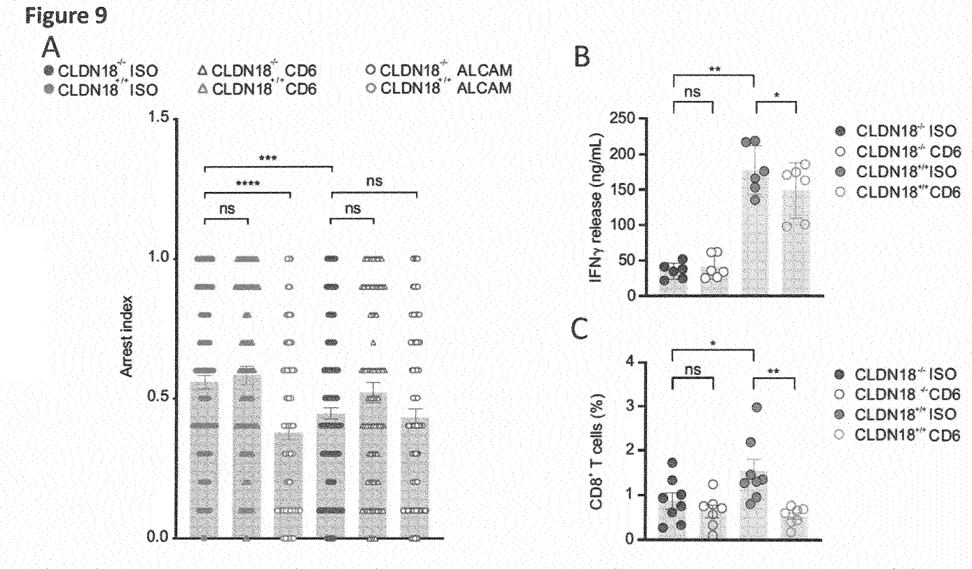

We isolated lipid rafts from monolayers of FC1242 cells expressing histidine (His)-tagged CLDN18 by collecting ten fractions after density gradient separation. Lipid rafts were identified by the presence of high amounts of cholesterol, phospholipids and caveolin associated with low actin levels (Cayrol, R. et al. (2008) Nat Immunol 9, 137-145); these features were mainly concentrated in fractions 3 and 4 of the density gradient separation. We observed that ALCAM and CLDN18 co-localized in these fractions, with a higher amount of ALCAM in lipid raft isolated from CLDN18^{+/+} than those from CLDN18^{-/-} clones, indicating that CLDN18 mediated ALCAM preferential localization to functional membrane micro domains (FIG. 5E-F). Notably, actin was identified in fraction 3 in CLDN18^{+/+} clones, whereas it was almost absent in the absence of CLDN18, further suggesting that CLDN18 could drive ALCAM localization in lipid rafts through actin. In accordance with this hypothesis, immunoprecipitation assay on FC1242-CLDN18-His followed by WB confirmed actin as CLDN18 intracellular interactor. We thus questioned whether CLDN18 effect on CTL-tumor cell interactions and IS formation was mediated by the ALCAM preferential accrual in lipid rafts. In a time-lapse in vitro setting, we observed that ALCAM-blockade (by antibody treatment) in CLDN18^{+/+} tumor lowered CTL contact time and arrest index to the level of CLDN18^{-/-} cells exposed to control isotype antibody (FIG. 5G). We then quantified IS formation in CTLs following interaction with CLDN18^{+/+} and CLDN18^{-/-} clones transfected with an ALCAM-targeting siRNA. Control siRNA did not affect neither ALCAM membrane expression nor IS formation in CTLs interacting with CLDN18^{+/+} cells, and both parameters were significantly higher compared to CLDN18^{-/-} clones. However, when ALCAM was silenced in CLDN18^{+/+} clones, both ALCAM membrane expression and IS formation in CTLs were no longer significantly different between CLDN18^{+/+} and CLDN18^{-/-} clones (FIG. 5H). We further strengthened these results by evaluating the number of TILs after ACT in CLDN18^{+/+} or CLDN18¹ tumor-bearing mice treated in vivo with either an ALCAM-blocking antibody or isotype control, demonstrating that ALCAM blockade disrupts the CLDN 18-conferred advantage in CTL homing to the tumor core (FIG. 51). Considering that ALCAM can affect CTL activity by both homotypic (ALCAM-ALCAM) and heterotypic (ALCAM-CD6) binding, we sought to investigate which axis supported CTL interaction with tumor cell. We evaluated the dynamics of OVA-specific CTL contacts with PDAC cells during either dual ALCAM-ALCAM and ALCAM-CD6 binding blockade (anti ALCAM) or selective CD6 blockade (by CD6 antagonist monoclonal antibody). ALCAM-ALCAM binding was crucial for initial T lymphocyte arrest on target cells, whereas ALCAM-CD6 interaction was negligible (FIG. 9A). Nonetheless, ALCAM-CD6 blockade did play a role in downstream signaling following 24-hour co-culture between T lymphocyte and tumor cells (FIG. 9B). In fact, interference with CD6 binding resulted in IFNγ release reduction only in CTLs co-cultured with CLDN18^{+/+} tumor cells (FIG. 9B). Accordingly, CD6 blockade in vivo impaired T lymphocyte infiltration in CLDN18^{+/+} tumors (FIG. 9C). These data suggest that homotypic ALCAM interactions are important for the early CTL-tumor cell contacts whereas binding of ALCAM to CD6 in T cells intervenes in later steps of cell retention and activation.

Collectively, in vitro and in vivo results highlight a role for CLDN18 in supporting CTLs interactions with tumor cells through the accrual of ALCAM on the tumor cell membrane.

### Example 6

### CLDN18 is a Prognostic Biomarker of OS in PDAC Patients.

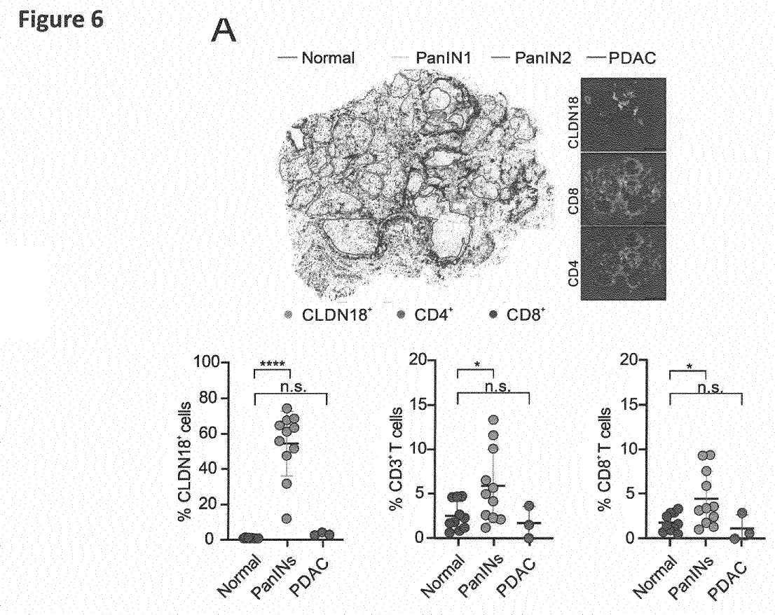

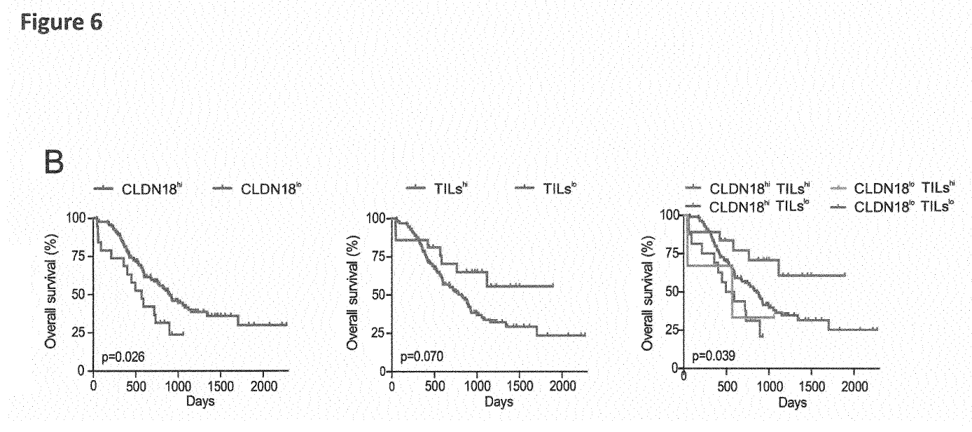

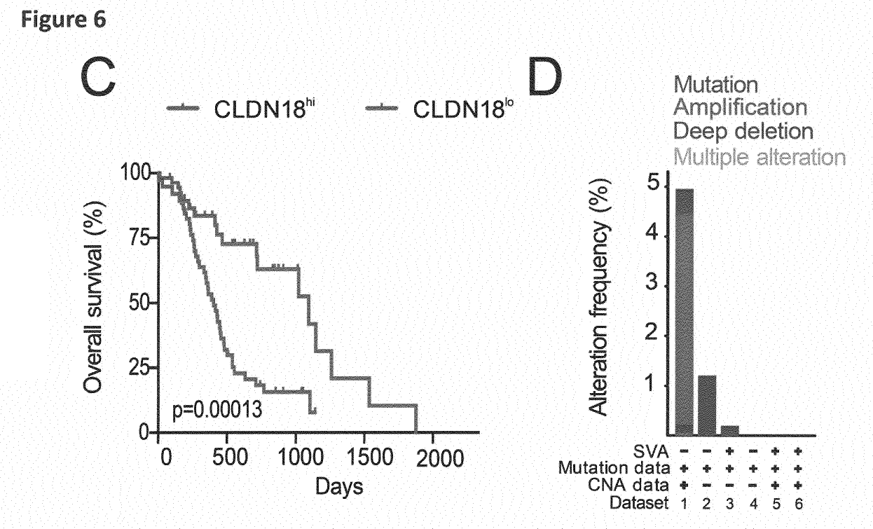

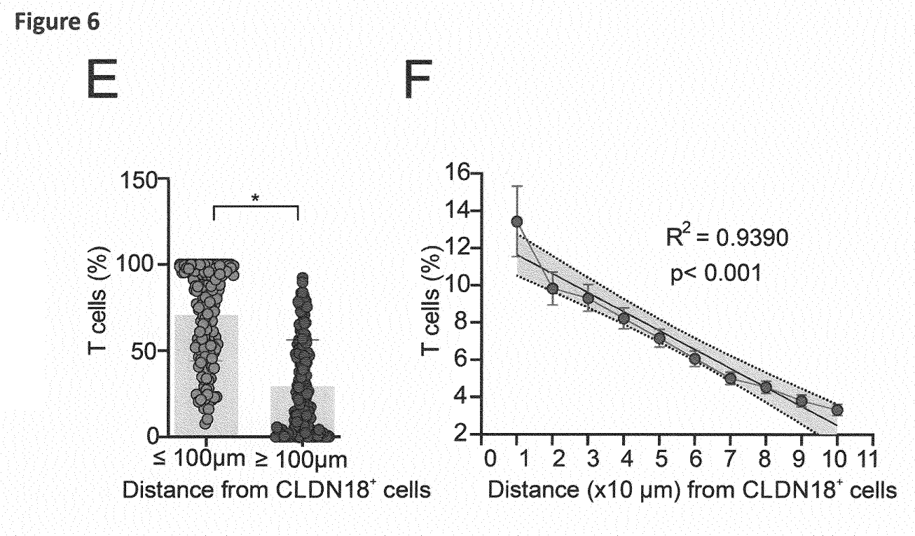

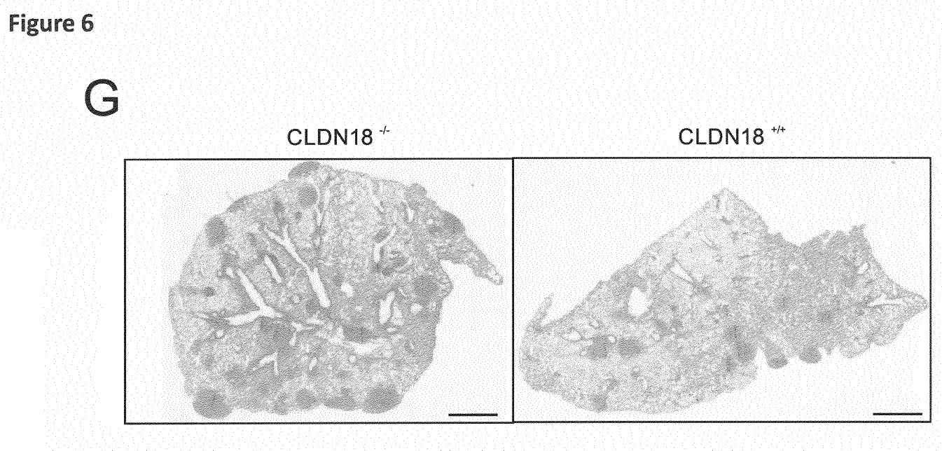

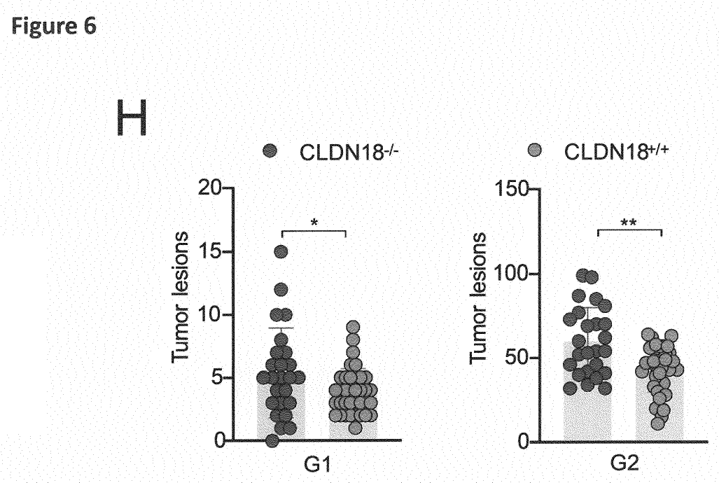

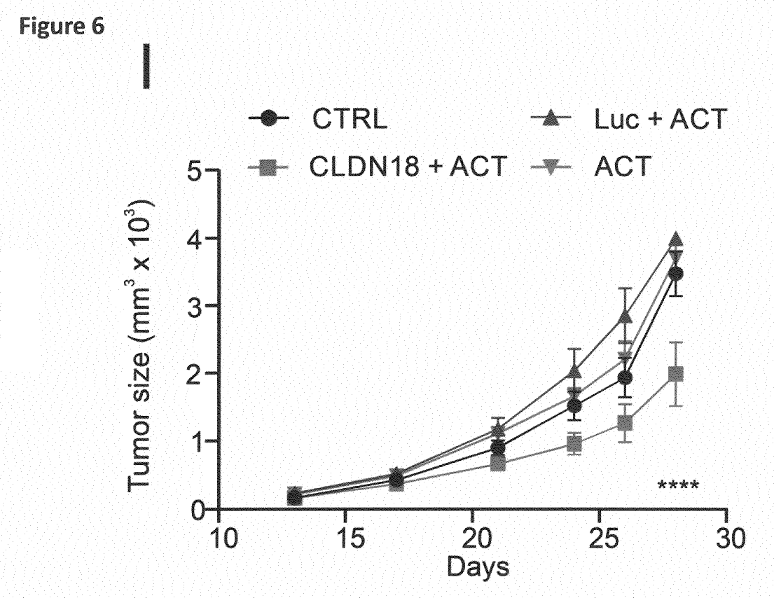

We evaluated primary tumor tissues from a cohort of patients characterized by early-stage pancreatic cancer. CLDN18, CD4 and CD8 multiple IF images were overlaid with H/E staining to integrate pathological information. Different tissue regions were classified by a pathologist as normal, early-stage neoplasia, named pancreatic intraepithelial neoplasia (PanIN), and PDAC (FIG. 6A). CLDN18, which is not expressed in normal tissue, was identified on epithelial cells of PanIN stages and downregulated in PDAC (FIG. 6A). Notably, CD3⁺ and CD8⁺ T lymphocyte infiltration followed a similar evolution with sustained infiltration in PanINs and exclusion in PDAC (FIG. 6A). We thus asked whether CLDN18 expression could serve as a biomarker of clinical outcome in PDAC patients. Double IHC for CLDN18 and CD3 in tissue microarray specimens from 148 PDAC patients confirmed CLDN18 as a prognostic factor stronger than TILs for better OS and DFS (FIG. 6B). This result was confirmed also by multivariable Cox analysis adjusting for the following risk factors: age, gender, residual tumor, and stage (Table 2).

TABLE 2
PDAC tissue microarray multivariate analysis (COX)
for CLDN18 and TILs
PDAC tissue microarray_COX

| Parameter | OS HR (95% CI) | p |
|---|---|---|
| CLDN18 | | 0.0316 |
| High vs low | 0.51 (0.28-0.94) | |
| TILs | | 0.095 |
| High vs low | 0.54 (0.62-1.11) | |
| Age | 1.74 (0.98-3.10) | 0.019 |
| Nonlinear | | 0.75 |
| Gender | | 0.86 |
| Male vs Female | 1.04 (0.67-1.62) | |
| Residual tumor | | 0.006 |
| R1 vs R0 | 1.91 (1.20-3.05) | |
| Staging | | 0.16 |
| IB vs IA | 94.7 (6.86e−06-1.3e+09) | |
| IIA vs IA | 10.22 (7.81e−07-1.34e+08) | |
| IIB vs IA | 60.65 (4.93-7.46 + 08) | |
| IV vs IA | 44.54 (3.38e−06-5.86e+08) | |
| CLDN18 and TILs (ref. | | 0.026 |
| CLDN18 low TILs low) | | |
| CLDN18 high TILs high | 0.26 (0.097-0.68) | |
| CLDN18 high TILs low | 0.59 (0.30-1.13) | |
| CLDN18 low TILs high | 1.72 (0.34-8.76) | |
| Age | 1.67 (0.93-3.02) | 0.022 |
| Nonlinear | | 0.83 |
| Gender | | 0.69 |
| Male vs Female | 1.1 (0.7-1.72) | |
| Residual tumor | | 0.005 |
| R1 vs R0 | 1.95 (1.21-3.14) | |
| Staging | | 0.10 |
| IB vs IA | 89.31 (6.45e−06-1.24e+09) | |
| IIA vs IA | 8.33 (6.32e−07-1.1e+08) | |
| IIB vs IA | 61.91 (5.02e−06-7.64e+08) | |
| IV vs IA | 42.98 (3.19e−06-5.56e+08) | |

Moreover, the presence of TILs in association with CLDN18 stratified the patients with improved clinical performance (FIG. 6B). We confirmed the relevance of these findings in independent cohorts of PDAC patients by investigating the ICGC dataset (Bailey, P. et al. (2016) Nature 531, 47-52) as shown in FIG. 6C and Table 3.

TABLE 3
Multivariate analysis (COX) for CLDN18 in Bailey et al. PDAC dataset
Bailey et al PDAC dataset_COX

| Parameter | OS HR (95% CI) | p |
|---|---|---|
| CLDN18 | | 0.002 |
| High vs low | 0.37 (0.19-0.71) | |
| Age | 1.33 (0.82-2.18) | 0.074 |
| Nonlinear | | 0.023 |
| Gender | | 0.33 |
| Male vs Female | 1.36 (0.73-2.55) | |

Notably, genome sequencing confirmed that CLDN18 downregulation in PDAC was barely dependent on genetic mutation and that Cldn18 was mainly regulated at the transcriptional level in pancreatic cancer cells (FIG. 6D). Finally, we evaluated the spatial distribution of TILs inside the tumor core in CLDN18-positive PDAC cases. TILs resulted preferentially located close to CLDN18⁺ tumor cells (FIG. 6E). More precisely, we unveiled an inverse correlation between TILs and the distance from CLDN18⁺ cells (FIG. 6F).

### Example 7

### CLDN18 and Cancer Immune Surveillance in Immunogenic Lung Adenocarcinoma.

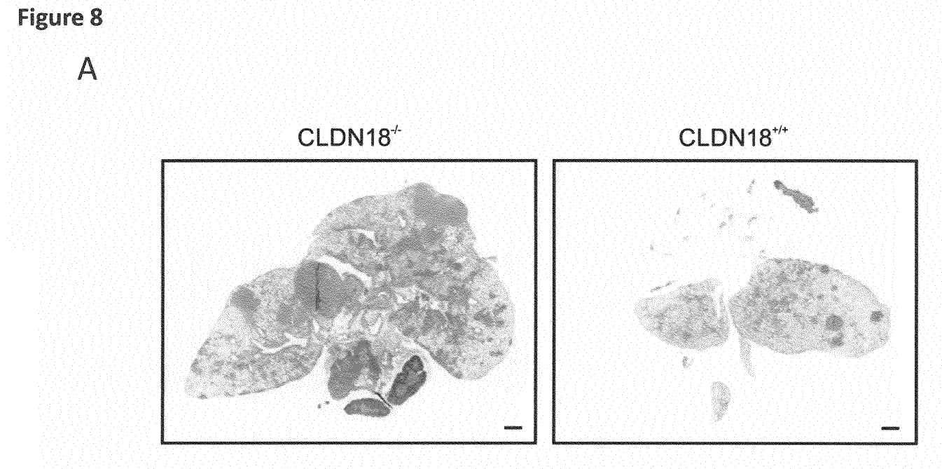

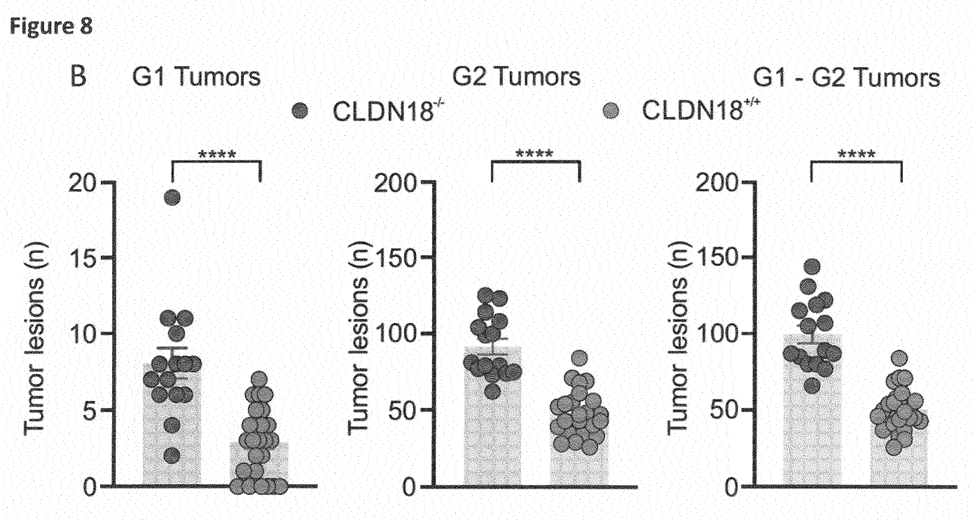

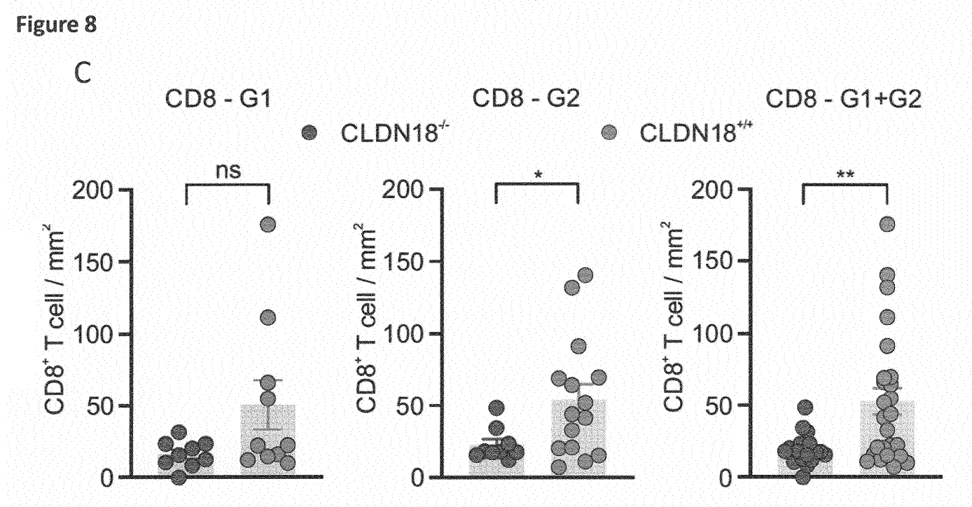

We finally evaluated whether CLDN18 loss could impair cancer immune surveillance fueling tumor immune escape. CLDN18.1 which is endogenously expressed in the lung, correlated with TILs in LUAD patients and contributed to stratify patients with improved OS (FIGS. 2F-H). We thus developed an autochthonous model of immunogenic lung adenocarcinoma (DuPage, M. et al. (2011) Cancer Cell 19, 72-85) in CLDN18-proficient and CLDN18-deficient genetic backgrounds. Briefly, we crossed LSL-Kras^{G12D/+} and LSL-Trp53^{R172H/+} with Cldn18^{flox/flox} mice to generate KPCldn^{fl/fl} mice. We activated the flox cassettes in epithelial lung cells by intranasal instillation of a CRE lentivirus engineered to express firefly luciferase fused with “SIINFEKL” immunodominant OVA peptide (DuPage et al., (2011) Cancer Cell 19, 72-85). In KPCldn^{+/+} and KPCldn^{fl/fl} mice, lung cell infection drove the activation of oncogenic Kras, Trp53 loss, and eventually CLDN18 deletion in floxed mice. Lungs were freshly isolated, and tumor burden was evaluated by bioluminescence imaging 18 weeks following virus instillation. A pathologist blindly defined the number of lesions, size and grade of each lesion/lung layer in a three-tier grading system (DuPage, M. et al. (2011) Cancer Cell 19, 72-85; Nikitin, A. Y. et al. (2004) Cancer Res 64, 2307-2316). Reference lung sections from CLDN18^{+/+} and CLDN18^{-/-} mice and the number of lesions classified according to the stage of progression are shown in FIG. 6G. Remarkably, we identified a higher number of grade 1-(G1, well differentiated, low grade) and grade 2-(G2, moderately differentiated, intermediate grade) tumor foci in CLDN18^{-/-} compared to CLDN18^{+/+} mice (FIG. 6H). These results were confirmed by establishing lung tumor lesions in CLDN18^{+/+} and CLDN18^{-/-} not expressing the SIINFEKL” immunodominant OVA peptide in an experiment wherein the CRE lentivirus does encompass the OVA peptide. Here reference lung sections from CLDN18^{+/+} and CLDN18^{-/-} mice and the number of lesions classified by stage of progression are shown in FIG. 8A. We identified a higher number of grade 1 and grade 2 tumor foci in CLDN18-compared with CLDN18^{+/+} mice (FIG. 8B). In accordance, CLDN18 loss was associated with impaired infiltration of CD8 CTLs in both grade 1 and grade 2 lesions (FIG. 8C). These results suggest that CLDN18 loss can decrease cancer immune surveillance and promote progression of early cancer lesions.

We also examined whether enforced CLDN18 expression in the tumor environment could support T lymphocyte antitumor activity. We first tested whether tumor cells could be engineered in vivo to express exogenous proteins following infection with a lentivirus encoding a CLDN18-GFP fusion construct. Three days after intratumoral delivery of different amounts of lentiviral particles in FC1242-tumor bearing mice, we observed a dose-dependent increase in lentivirus-infected (i.e., GFP⁺) cells. Most transduced cells were tumor cells (approximately 70%) and leukocytes. After demonstrating that this strategy successfully delivered CLDN18 within the tumor environment, we in-situ injected either GFP-CLDN18 or luciferase (control) lentiviruses into immunodeficient mice bearing cold FC1242-OVA tumors, followed by ACT three days later. Lentivirus infection alone did not bring any benefit compared to the control; conversely, ectopic CLDN18-expression significantly improved ACT efficacy.

These data indicate that CLDN18 favors the interaction of CTLs with transformed epithelial cells, promoting their local activation and cytokine release. Forced and targeted CLDN18 expression may thus offer therapeutic prospects to modulate favorably tumor hotness and cancer immune surveillance.

### Example 8

### CLDN18 Exogenous Regulation In Vitro.

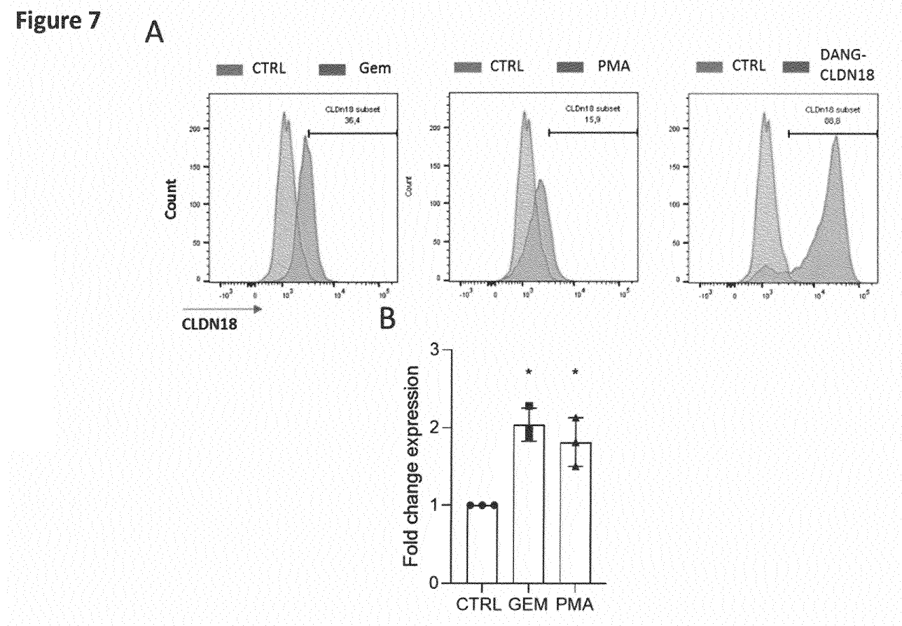

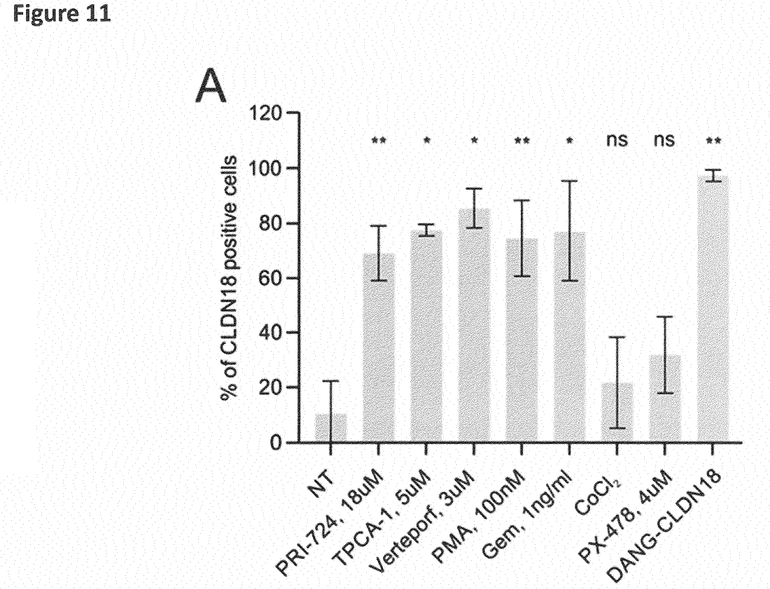

The human pancreatic adenocarcinoma cell line DANG expressing CLDN18 (DANG_CLDN18) or not expressing CLDN18 (DANG) was cultured in RPMI media supplemented with 10% FBS, L-Glutamin, Hepes. Cells were treated for 48 hours with Gemcitabine (Gem) or Phorbol 12-myristate 13-acetate (PMA) or left untreated. DANG-CLDN18 were used as positive control. Cells were collected, fixed and permeabilized, incubated with a rabbit anti mouse CLDN18 antibody, and with an APC-conjugated secondary anti rabbit (both from ThermoFisher Scientific). As shown in FIG. 7, Gemcitabine and PMA induced CLDN18 expression in DANG cells. Similar observations are made in an extended experiment including PRI-724, TPCA-1, Verteporfin, CoCL2, PX-478 and PMA, where with the exemption of CoCL2, PX-478 all induced CLDN18 expression in DANG cells as shown in FIG. 11.

1. A method for determining the ability of a cancer patient to mount a natural or immunotherapy-induced immune response against the cancer comprising determining the expression level of claudin 18 (CLDN18) in a sample obtained from the patient.

2. The method of claim 1, wherein the immune response involves anti-tumor T cells.

3. The method of claim 1 or 2, wherein the immune response comprises targeting an antigen other than CLDN18.

4. The method of claim 3, wherein the antigen other than CLDN18 is a tumor antigen.

5. The method of any one of claims 1 to 4, wherein expression of CLDN18 indicates that the patient is able to mount a natural or immunotherapy-induced immune response against the cancer.

6. The method of any one of claims 1 to 5, wherein a level of CLDN18 at or above a reference level indicates that the patient is able to mount a natural or immunotherapy-induced immune response against the cancer.

7. The method of any one of claims 1 to 6, which is for determining the survival perspective of the cancer patient.

8. The method of claim 7, wherein expression of CLDN18 indicates that the patient has a favorable survival perspective.

9. The method of claim 7 or 8, wherein a level of CLDN18 at or above a reference level indicates that the patient has a favorable survival perspective.

10. The method of any one of claims 1 to 9, which is for determining the responsiveness of the cancer patient to immunotherapy.

11. The method of any one of claims 1 to 10, wherein the immunotherapy aims at inducing an immune response involving anti-tumor T cells.

12. The method of any one of claims 1 to 11, wherein the immunotherapy aims at enhancing T cell tumor infiltration and/or the number and/or activity of anti-tumor T cells.

13. The method of any one of claims 1 to 12, wherein the immunotherapy is selected from the group consisting of immune checkpoint blockade, T cell stimulation, cancer vaccines, oncolytic viruses, antigen-presenting cell activation, adoptive T cell therapies and CAR T cell therapies.

14. The method of any one of claims 1 to 13, wherein the immunotherapy comprises targeting an antigen other than CLDN18.

15. The method of claim 14, wherein the antigen other than CLDN18 is a tumor antigen.

16. The method of any one of claims 10 to 15, wherein expression of CLDN18 indicates that the patient responds to immunotherapy.

17. The method of any one of claims 10 to 16, wherein a level of CLDN18 at or above a reference level indicates that the patient responds to immunotherapy.

18. A method for determining the survival perspective of a cancer patient comprising determining the expression level of claudin 18 (CLDN18) in a sample obtained from the patient.

19. The method of claim 18, wherein expression of CLDN18 indicates that the patient has a favorable survival perspective.

20. The method of claim 18 or 19, wherein a level of CLDN18 at or above a reference level indicates that the patient has a favorable survival perspective.

21. A method for determining the responsiveness of a cancer patient to immunotherapy comprising determining the expression level of claudin 18 (CLDN18) in a sample obtained from the patient.

22. The method of claim 21, wherein the immunotherapy aims at inducing an immune response involving anti-tumor T cells.

23. The method of claim 21 or 22, wherein the immunotherapy aims at enhancing T cell tumor infiltration and/or the number and/or activity of anti-tumor T cells.

24. The method of any one of claims 21 to 23, wherein the immunotherapy is selected from the group consisting of immune checkpoint blockade, T cell stimulation, cancer vaccines, oncolytic viruses, antigen-presenting cell activation, adoptive T cell therapies and CAR T cell therapies.

25. The method of any one of claims 21 to 24, wherein the immunotherapy comprises targeting an antigen other than CLDN18.

26. The method of claim 25, wherein the antigen other than CLDN18 is a tumor antigen.

27. The method of any one of claims 21 to 26, wherein expression of CLDN18 indicates that the patient responds to immunotherapy.

28. The method of any one of claims 21 to 27, wherein a level of CLDN18 at or above a reference level indicates that the patient responds to immunotherapy.

29. The method of any one of claims 1 to 28, wherein the sample comprises cancer cells.

30. The method of any one of claims 1 to 29, wherein the sample comprises a tumor biopsy.

31. A method for treating a cancer patient, the method comprising:
(i) determining the expression level of claudin 18 (CLDN18) in a sample obtained from the patient and
(ii) administering an immunotherapy to the patient, if the patient is found to be eligible in (i).

32. The method of claim 31, wherein, if CLDN18 is expressed, the immunotherapy is administered to the patient.

33. The method of claim 31 or 32, wherein, if the level of CLDN18 is at or above a reference level, the immunotherapy is administered to the patient.

34. The method of any one of claims 31 to 33, wherein, if CLDN18 is not expressed, the immunotherapy is not administered to the patient or is administered in combination with an agent stabilizing or increasing expression of CLDN18.

35. The method of any one of claims 31 to 34, wherein, if the level of CLDN18 is below a reference level, the immunotherapy is not administered to the patient or is administered in combination with an agent stabilizing or increasing expression of CLDN18.

36. A method for treating a cancer patient, the method comprising:
(i) determining that a sample obtained from the patient has an expression level of claudin 18 (CLDN18) at or above a reference level and
(ii) administering an immunotherapy to the patient.

37. The method of any one of claims 31 to 36, wherein the immunotherapy aims at inducing an immune response involving anti-tumor T cells.

38. The method any one of claims 31 to 37, wherein the immunotherapy aims at enhancing T cell tumor infiltration and/or the number and/or activity of anti-tumor T cells.

39. The method of any one of claims 31 to 38, wherein the immunotherapy is selected from the group consisting of immune checkpoint blockade, T cell stimulation, cancer vaccines, oncolytic viruses, antigen-presenting cell activation, adoptive T cell therapies and CAR T cell therapies.

40. The method of any one of claims 31 to 39, wherein the immunotherapy comprises targeting an antigen other than CLDN18.

41. The method of claim 40, wherein the antigen other than CLDN18 is a tumor antigen.

42. The method of any one of claims 31 to 41, wherein the sample comprises cancer cells.

43. The method of any one of claims 33 to 42, wherein the sample comprises a tumor biopsy.

44. A method for enhancing the ability of a cancer patient to mount a natural or immunotherapy-induced immune response against the cancer comprising administering an agent stabilizing or increasing expression of claudin 18 (CLDN18).

45. The method of claim 44, wherein the immune response involves anti-tumor T cells.

46. The method of claim 44 or 45, wherein the immune response comprises targeting an antigen other than CLDN18.

47. The method of claim 46, wherein the antigen other than CLDN18 is a tumor antigen.

48. The method of any one of claims 44 to 47, wherein the immunotherapy aims at inducing an immune response involving anti-tumor T cells.

49. The method of any one of claims 44 to 48, wherein the immunotherapy aims at enhancing T cell tumor infiltration and/or the number and/or activity of anti-tumor T cells.

50. The method of any one of claims 44 to 49, wherein the immunotherapy is selected from the group consisting of immune checkpoint blockade, T cell stimulation, cancer vaccines, oncolytic viruses, antigen-presenting cell activation, adoptive T cell therapies and CAR T cell therapies.

51. The method of any one of claims 44 to 50, wherein the immunotherapy comprises targeting an antigen other than CLDN18.

52. The method of claim 51, wherein the antigen other than CLDN18 is a tumor antigen.

53. A method for enhancing the responsiveness of a cancer patient to immunotherapy comprising administering an agent stabilizing or increasing expression of claudin 18 (CLDN18).

54. A method for treating a cancer patient, the method comprising:
(i) administering an agent stabilizing or increasing expression of claudin 18 (CLDN18) and
(ii) administering an immunotherapy to the patient.

55. The method of claim 53 or 54, wherein the immunotherapy aims at inducing an immune response involving anti-tumor T cells.

56. The method of any one of claims 53 to 55, wherein the immunotherapy aims at enhancing T cell tumor infiltration and/or the number and/or activity of anti-tumor T cells.

57. The method of any one of claims 53 to 56, wherein the immunotherapy is selected from the group consisting of immune checkpoint blockade, T cell stimulation, cancer vaccines, oncolytic viruses, antigen-presenting cell activation, adoptive T cell therapies and CAR T cell therapies.

58. The method of any one of claims 53 to 57, wherein the immunotherapy comprises targeting an antigen other than CLDN18.

59. The method of claim 58, wherein the antigen other than CLDN18 is a tumor antigen.

60. A method for treating a cancer patient, the method comprising:
(i) determining the expression level of claudin 18 (CLDN18) in a tumor biopsy sample obtained from the patient
(ii) comparing the expression level of CLDN18 to a reference, and
(iii) administering an immunotherapy to the patient when the expression level of CLDN18 is higher than the reference.

61. A method for treating a cancer patient, the method comprising administering immunotherapy to a patient having been determined to have a tumor that expresses claudin 18 (CLDN18).

62. A method for treating a cancer patient, the method comprising administering immunotherapy to a patient having been determined to have a tumor that expresses claudin 18 (CLDN18) at a level higher than a reference.

63. A method of increasing therapeutic T-cell migration to a tumor in the patient, the method comprising administering to the patient an agent that stabilizes or increases expression of claudin 18 (CLDN18).

64. A method for treating a tumor in a patient, the method comprising administering to the patient immunotherapy in combination with an agent that stabilizes or increases expression of claudin 18 (CLDN18) if the patient has been determined to have a tumor that does not express CLDN18 or that expresses CLDN18 at a level lower than a reference.

65. The method of claim 64, wherein the agent that stabilizes or increases expression of CLDN18 is administered prior to, simultaneously with or subsequently to the immunotherapy.

66. The method of any one of claims 1 to 65, wherein the cancer comprises pancreatic cancer.

67. The method of any one of claims 1 to 66, wherein the cancer comprises lung cancer.

68. The method of any one of claims 1 to 67, wherein the cancer comprises lung adenocarcinoma.


SEQUENCE LISTING (3 sequences)

SEQ ID NO: 1 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 2 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 3 - protein, 8 aa, synthetic construct
SIINFEKL